(19) United States
(12) Patent Application Publication
(10) Pub. No.: US 2025/0354183 A1
(43) Pub. Date: Nov. 20, 2025

(54) METHOD FOR PRODUCING L-GLUTAMIC ACID

(71) Applicant: Ajinomoto Co., Inc., Tokyo (JP)

(72) Inventors: Daiki YAHAGI, Kanagawa (JP); Kazuyuki HAYASHI, Kanagawa (JP); Misato OKAMOTO, Kanagawa (JP); Miyoko MAEDA, Kanagawa (JP); Morito SHIMOMURA, Kanagawa (JP); Yoshinori TAJIMA, Kanagawa (JP)

(73) Assignee: Ajinomoto Co., Inc., Tokyo (JP)

(21) Appl. No.: 19/288,682

(22) Filed: Aug. 1, 2025

Related U.S. Application Data
(63) Continuation of application No. PCT/JP2024/003374, filed on Feb. 1, 2024.

(30) Foreign Application Priority Data
Feb. 1, 2023 (JP) 2023-014208

(51) Int. Cl. C12P 13/14

(52) U.S. Cl. CPC C12P 13/14

(57) ABSTRACT

A method for producing L-glutamic acid is provided. The L-glutamic acid is produced by culturing L-glutamic acid-producing strain of Corynebacterium casei under oxygen limitation conditions.

This application is a Continuation of, and claims priority under 35 U.S.C. § 120 to, International Patent Application No. PCT/JP2024/003374, filed Feb. 1, 2024, and claims priority therethrough under 35 U.S.C. § 119 to Japanese Patent Application No. 2023-014208, filed Feb. 1, 2023, the entireties of which are incorporated by reference herein. The Sequence Listing filed electronically herewith in ST.26 xml format named P231901WO_seql.xml, 6,076 bytes, generated on Jul. 9, 2025 is also incorporated by reference.

### BACKGROUND

### Technical Field

The present invention relates to a method for producing L-glutamic acid by fermentation using bacteria. L-glutamic acid is industrially useful as a raw material for seasonings and so forth.

### Background Art

L-amino acids such as L-glutamic acid are industrially produced by, for example, fermentation methods using microorganisms such as bacteria having an L-amino acid-producing ability (Non-patent document 1). As such microorganisms, for example, strains isolated from nature and mutant strains thereof have been used. Furthermore, an L-amino acid-producing ability of microorganisms can be improved using recombinant DNA techniques. In the production of L-amino acids such as L-glutamic acid by fermentation methods, the cultivation of microorganisms is performed aerobically (Patent document 1).

### PRIOR ART REFERENCES

### Patent Documents

[Patent document 1] WO2022/092018

### Non-Patent Documents

[Non-patent document 1] Akashi, K. et al., Amino Acid Fermentation. Japan Scientific Societies Press, p. 195 to 215, 1986.

### SUMMARY

An aspect of the present invention is to develop a novel technique for improving an L-glutamic acid-producing ability of bacteria, and thereby provide a method for efficiently producing L-glutamic acid.

In order to achieve the aforementioned aspect, the present inventors have discovered that by culturing an L-glutamic acid-producing strain of Corynebacterium casei under oxygen-limited conditions, it is possible to improve L-glutamic acid production by the strain, thereby completing the present invention. Specifically, unlike typical Coryneform bacteria such as Corynebacterium glutamicum, which produce high levels of L-glutamic acid under aerobic conditions, an aspect of the present invention was made based on the surprising finding that Corynebacterium casei produces high levels of L-glutamic acid under oxygen-limited conditions.

It is an aspect of the present invention to provide a method for producing L-glutamic acid, the method comprising: a step of culturing a coryneform bacterium having an L-glutamic acid-producing ability in a culture medium to obtain a culture product containing L-glutamic acid, wherein the bacterium is Corynebacterium casei, and wherein oxygen limitation is implemented during said step.

It is a further aspect of the present invention to provide the method as described above, wherein the oxygen limitation is implemented so that the culture condition is a microaerobic condition.

It is a further aspect of the present invention to provide the method as described above, wherein the step comprises a culture period under an aerobic condition followed by a culture period under a microaerobic condition.

It is a further aspect of the present invention to provide the method as described above, wherein dissolved oxygen concentration in the culture medium during the period in which the oxygen limitation is implemented is less than 0.18 ppm.

It is a further aspect of the present invention to provide the method as described above, wherein oxygen consumption rate during the period in which the oxygen limitation is implemented is from 0.075 to 0.4 mol (O₂)/min/mL per OD620.

It is a further aspect of the present invention to provide the method as described above, wherein dissolved oxygen concentration in the culture medium during a period in which the oxygen limitation is not implemented is 0.18 ppm or more.

It is a further aspect of the present invention to provide the method as described above, wherein the oxygen limitation is implemented at a point when OD620 is 30 or more.

It is a further aspect of the present invention to provide the method as described above, wherein the length of the period in which the oxygen limitation is implemented is 1 hour or more.

It is a further aspect of the present invention to provide the method as described above, further comprising a step of increasing temperature of the culture medium during said step.

It is a further aspect of the present invention to provide the method as described above, wherein an amount of said increase is from 1° C. to 12° C.

It is a further aspect of the present invention to provide the method as described above, wherein the temperature of the culture medium after said increase is from 32° C. to 40° C.

It is a further aspect of the present invention to provide the method as described above, wherein the temperature of the culture medium prior to said increase is from 20° C. to 32° C.

It is a further aspect of the present invention to provide the method as described above, wherein the increase is implemented at a point when OD620 is 30 or more.

It is a further aspect of the present invention to provide the method as described above, wherein the length for which the temperature after said increase is maintained is 1 hour or more.

It is a further aspect of the present invention to provide the method as described above, wherein the L-glutamic acid is L-glutamic acid in a free form, sodium L-glutamate, potassium L-glutamate, ammonium L-glutamate, or a mixture thereof.

It is a further aspect of the present invention to provide the method as described above, wherein the L-glutamic acid is produced as a composition containing the L-glutamic acid.

It is a further aspect of the present invention to provide the method as described above, wherein the composition is a dried product of the culture product or a dried product of the supernatant of the culture product.

It is a further aspect of the present invention to provide the method as described above, further comprising a step of collecting the L-glutamic acid from the culture product.

It is a further aspect of the present invention to provide the method as described above, wherein the bacterium has one or more mutations selected from the mutations shown in Table 1 described later.

According to the present invention, L-glutamic acid can be efficiently produced.

### BRIEF DESCRIPTION OF THE DRAWINGS

FIG. 1 shows culture results (PL electrode readings) of C. casei JCM 12072 (wild-type strain) under the conditions of temperature elevation and/or oxygen limitation.

FIG. 2 shows culture results (OD620 (×101)) of C. casei JCM 12072 (wild-type strain) under the conditions of temperature elevation and/or oxygen limitation.

FIG. 3 shows culture results (Glu accumulation) of C. casei JCM 12072 (wild-type strain) under the conditions of temperature elevation and/or oxygen limitation.

FIG. 4 shows culture results (Residual Sugar) of C. casei JCM 12072 (wild-type strain) under the conditions of temperature elevation and/or oxygen limitation.

FIG. 5 shows culture results (acetic acid accumulation) of C. casei JCM 12072 (wild-type strain) under the conditions of temperature elevation and/or oxygen limitation.

FIG. 6 shows culture results (lactic acid accumulation) of C. casei JCM 12072 (wild-type strain) under the conditions of temperature elevation and/or oxygen limitation.

FIG. 7 shows culture results (succinic acid accumulation) of C. casei JCM 12072 (wild-type strain) under the conditions of temperature elevation and/or oxygen limitation.

FIG. 8 shows culture results (α-ketoglutaric acid accumulation) of C. casei JCM 12072 (wild-type strain) under the conditions of temperature elevation and/or oxygen limitation.

FIG. 9 shows culture results (PL electrode readings) of C. casei AJ111891 (NITE BP-03688) under the conditions of temperature elevation and/or oxygen limitation.

FIG. 10 shows culture results (OD620 (×101)) of C. casei AJ111891 (NITE BP-03688) under the conditions of temperature elevation and/or oxygen limitation.

FIG. 11 shows culture results (Glu accumulation) of C. casei AJ111891 (NITE BP-03688) under the conditions of temperature elevation and/or oxygen limitation.

FIG. 12 shows culture results (Residual Sugar) of C. casei AJ111891 (NITE BP-03688) under the conditions of temperature elevation and/or oxygen limitation.

FIG. 13 shows culture results (α-ketoglutaric acid accumulation) of C. casei AJ111891 (NITE BP-03688) under the conditions of temperature elevation and/or oxygen limitation.

FIG. 14 shows culture results (oxygen consumption rate; Rab) of C. casei AJ111891 (NITE BP-03688) under the conditions of oxygen limitation.

FIG. 15 shows culture results (PL electrode readings) of C. casei AJ111891 (NITE BP-03688) under the conditions of oxygen limitation.

FIG. 16 shows culture results (OD620 (×101)) of C. casei AJ111891 (NITE BP-03688) under the conditions of oxygen limitation.

FIG. 17 shows culture results (Glu accumulation) of C. casei AJ111891 (NITE BP-03688) under the conditions of oxygen limitation.

FIG. 18 shows culture results (oxygen consumption rate; Rab) of C. casei AJ111891 (NITE BP-03688) under the conditions of temperature elevation and oxygen limitation.

FIG. 19 shows culture results (PL electrode readings) of C. casei AJ111891 (NITE BP-03688) under the conditions of temperature elevation and oxygen limitation.

FIG. 20 shows culture results (OD620 (×101)) of C. casei AJ111891 (NITE BP-03688) under the conditions of temperature elevation and oxygen limitation.

FIG. 21 shows culture results (Glu accumulation) of C. casei AJ111891 (NITE BP-03688) under the conditions of temperature elevation and oxygen limitation.

FIG. 22 shows the correlation between weight yield of Glu relative to sugar and oxygen consumption rate (Rab or Rab/Cell-OD620) under the conditions of temperature elevation and/or oxygen limitation in C. casei AJ111891 (NITE BP-03688).

FIG. 23 shows culture results (PL electrode readings) of C. casei A-013 (NITE BP-03806) under the conditions of temperature elevation and/or oxygen limitation.

FIG. 24 shows culture results (OD620 (×101)) of C. casei A-013 (NITE BP-03806) under the conditions of temperature elevation and/or oxygen limitation.

FIG. 25 shows culture results (Glu accumulation) of C. casei A-013 (NITE BP-03806) under the conditions of temperature elevation and/or oxygen limitation.

FIG. 26 shows culture results (Residual Sugar) of C. casei A-013 (NITE BP-03806) under the conditions of temperature elevation and/or oxygen limitation.

FIG. 27 shows culture results (acetic acid accumulation) of C. casei A-013 (NITE BP-03806) under the conditions of temperature elevation and/or oxygen limitation.

FIG. 28 shows culture results (lactic acid accumulation) of C. casei A-013 (NITE BP-03806) under the conditions of temperature elevation and/or oxygen limitation.

FIG. 29 shows culture results (succinic acid accumulation) of C. casei A-013 (NITE BP-03806) under the conditions of temperature elevation and/or oxygen limitation.

FIG. 30 shows culture results (α-ketoglutaric acid accumulation) of C. casei A-013 (NITE BP-03806) under the conditions of temperature elevation and/or oxygen limitation.

FIG. 31 shows culture results (PL electrode readings) of C. casei WT+pVK9-ybjL under the conditions of temperature elevation and/or oxygen limitation.

FIG. 32 shows culture results (OD620 (×101)) of C. casei WT+pVK9-ybjL under the conditions of temperature elevation and/or oxygen limitation.

FIG. 33 shows culture results (Glu accumulation) of C. casei WT+pVK9-ybjL under the conditions of temperature elevation and/or oxygen limitation.

FIG. 34 shows culture results (Residual Sugar) of C. casei WT+pVK9-ybjL under the conditions of temperature elevation and/or oxygen limitation.

FIG. 35 shows culture results (lactic acid accumulation) of C. casei WT+pVK9-ybjL under the conditions of temperature elevation and/or oxygen limitation.

FIG. 36 shows culture results (succinic acid accumulation) of C. casei WT+pVK9-ybjL under the conditions of temperature elevation and/or oxygen limitation.

FIG. 37 shows culture results (PL electrode readings) of C. glutamicum ATCC 13869 strain (wild-type strain) under the conditions of temperature elevation and/or oxygen limitation.

FIG. 38 shows culture results (OD620 (×101)) of C. glutamicum ATCC 13869 strain (wild-type strain) under the conditions of temperature elevation and/or oxygen limitation.

FIG. 39 shows culture results (Glu accumulation) of C. glutamicum ATCC 13869 strain (wild-type strain) under the conditions of temperature elevation and/or oxygen limitation.

FIG. 40 shows culture results (Residual Sugar) of C. glutamicum ATCC 13869 strain (wild-type strain) under the conditions of temperature elevation and/or oxygen limitation.

FIG. 41 shows culture results (lactic acid accumulation) of C. glutamicum ATCC 13869 strain (wild-type strain) under the conditions of temperature elevation and/or oxygen limitation.

FIG. 42 shows culture results (PL electrode readings) of C. glutamicum ATCC 13869-L30 strain under the conditions of temperature elevation and/or oxygen limitation.

FIG. 43 shows culture results (OD620 (×101)) of C. glutamicum ATCC 13869-L30 strain under the conditions of temperature elevation and/or oxygen limitation.

FIG. 44 shows culture results (Glu accumulation) of C. glutamicum ATCC 13869-L30 strain under the conditions of temperature elevation and/or oxygen limitation.

FIG. 45 shows culture results (Residual Sugar) of C. glutamicum ATCC 13869-L30 strain under the conditions of temperature elevation and/or oxygen limitation.

FIG. 46 shows culture results (acetic acid accumulation) of C. glutamicum ATCC 13869-L30 strain under the conditions of temperature elevation and/or oxygen limitation.

FIG. 47 shows culture results (lactic acid accumulation) of C. glutamicum ATCC 13869-L30 strain under the conditions of temperature elevation and/or oxygen limitation.

FIG. 48 shows culture results (succinic acid accumulation) of C. glutamicum ATCC 13869-L30 strain under the conditions of temperature elevation and/or oxygen limitation.

FIG. 49 shows culture results (α-ketoglutaric acid accumulation) of C. glutamicum ATCC 13869-L30 strain under the conditions of temperature elevation and/or oxygen limitation.

### DETAILED DESCRIPTION OF EXEMPLARY EMBODIMENTS

Hereinafter, the present invention will be explained in detail.

The method described herein is a method for producing L-glutamic acid, the method comprising: a step of culturing a coryneform bacterium having an L-glutamic acid-producing ability in a culture medium, wherein the bacterium is Corynebacterium casei, and wherein oxygen limitation is implemented during said step.

The aforementioned bacterium is also referred to as “L-glutamic acid producing bacterium”.

### <1> L-Glutamic Acid-Producing Bacterium

The L-glutamic acid-producing bacterium described herein is a coryneform bacterium having an L-glutamic acid-producing ability. The L-glutamic acid-producing bacterium described herein is specifically Corynebacterium casei having an L-glutamic acid-producing ability.

The “bacterium having an L-glutamic acid-producing ability” refers to a bacterium having an ability to generate and accumulate L-glutamic acid in a culture medium and/or cells of the bacterium in such a degree that the L-glutamic acid can be collected, when the bacterium is cultured in the culture medium. The bacterium having the L-glutamic acid-producing ability may be a bacterium that is able to accumulate L-glutamic acid in a culture medium preferably in an amount of 0.5 g/L or more, or more preferably 1.0 g/L or more.

The term “glutamic acid” refers to L-glutamic acid, unless otherwise stated. The term “L-glutamic acid” refers to L-glutamic acid in a free form, a salt thereof, or a mixture thereof, unless otherwise stated. The term “free form” refers to a compound that is not in a salt form. Examples of the salt include sulphate, hydrochloride, carbonate, ammonium, sodium and potassium salts. Examples of the salt of L-glutamic acid include, specifically, sodium L-glutamate (such as monosodium L-glutamate; MSG), potassium L-glutamate (such as monopotassium L-glutamate) and ammonium L-glutamate (such as monoammonium L-glutamate). In other words, examples of L-glutamic acid may be, specifically, L-glutamic acid in a free form, sodium L-glutamate (such as monosodium L-glutamate; MSG), potassium L-glutamate (such as monopotassium L-glutamate), ammonium L-glutamate (such as monoammonium L-glutamate), or a mixture thereof.

The L-glutamic acid-producing bacterium may be a bacterium inherently having the L-glutamic acid-producing ability or may be a bacterium modified so that it has the L-glutamic acid-producing ability. The L-glutamic acid-producing bacterium can be obtained by imparting the L-glutamic acid-producing ability to a Corynebacterium casei strain, or by enhancing the L-glutamic acid-producing ability of a Corynebacterium casei strain. Examples of Corynebacterium casei strains that can be used as the L-glutamic acid-producing bacteria or parental strains for constructing the bacteria thereof include Corynebacterium casei JCM 12072. In other words, examples of the L-glutamic acid-producing bacteria include modified strains derived from or native to Corynebacterium casei JCM 12072.

These strains of Corynebacterium casei are available from, for example, the American Type Culture Collection (Address: 12301 Parklawn Drive, Rockville, Maryland 20852, P.O. Box 1549, Manassas, VA 20108, United States of America). That is, registration numbers are given to these strains, and the strains can be ordered by using these registration numbers (refer to http://www.atcc.org/). The registration numbers of the strains are listed in the catalogue of the American Type Culture Collection. These strains of Corynebacterium casei can also be obtained from, for example, the depositories at which the strains were deposited.

Methods for imparting or enhancing L-glutamic acid-producing ability are not particularly limited. Examples of the methods for imparting or enhancing L-glutamic acid-producing ability include, for example, known methods. Imparting or enhancing L-glutamic acid-producing ability can be performed by, for example, mutagenesis methods or genetic engineering techniques. Examples of the methods for imparting or enhancing L-glutamic acid-producing ability are disclosed, for example, in WO2006/070944 and WO2015/060391.

Methods for imparting or enhancing L-glutamic acid-producing ability can also be performed by, for example, the procedures described in the Examples hereinafter.

Examples of the methods for imparting or enhancing L-glutamic acid-producing ability include a method for modifying a bacterium so that the activity or activities of one or more kinds of enzymes selected from the L-glutamic acid biosynthesis enzymes are enhanced. Examples of such enzymes include, but not particularly limited to, glutamate dehydrogenase (gdhA), glutamine synthetase (glnA), glutamate synthase (gltBD), isocitrate dehydrogenase (icdA), aconitate hydratase (acnA, acnB), citrate synthase (gltA), methylcitrate synthase (prpC), pyruvate carboxylase (pyc), pyruvate dehydrogenase (aceEF, lpdA), pyruvate kinase (pykA, pykF), phosphoenolpyruvate synthase (ppsA), enolase (eno), phosphoglyceromutase (pgmA, pgmI), phosphoglycerate kinase (pgk), glyceraldehyde-3-phosphate dehydrogenase (gapA), triose phosphate isomerase (tpiA), fructose bisphosphate aldolase (fbp), glucose phosphate isomerase (pgi), 6-phosphogluconate dehydratase (edd), 2-keto-3-deoxy-6-phosphogluconate aldolase (eda), and transhydrogenase (pntAB). Shown in the parentheses after the names of the enzymes are examples of genes encoding the enzymes (the same shall apply to the same occasions hereinafter). It is preferable to enhance the activity or activities of one or more kinds of enzymes selected from, for example, glutamate dehydrogenase, citrate synthase, phosphoenol pyruvate carboxylase, and methylcitrate synthase, among these enzymes.

Examples of the methods for imparting or enhancing L-glutamic acid-producing ability also include a method for modifying a bacterium so that the bacterium has a reduced activity or activities of one or more kinds of enzymes (including enzymes involved in the degradation of L-glutamic acid) selected from the enzymes that catalyze a reaction branching away from the biosynthesis pathway of L-glutamic acid to generate a compound other than L-glutamic acid. Examples of such enzymes include, but not particularly limited to, isocitrate lyase (aceA), α-ketoglutarate dehydrogenase (sucA, odhA), acetolactate synthase (ilvI), formate acetyltransferase (pfl), lactate dehydrogenase (ldh), alcohol dehydrogenase (adh), glutamate decarboxylase (gadAB), and succinate dehydrogenase (sdhABCD). It is preferable to reduce or delete, for example, the α-ketoglutarate dehydrogenase activity, among these enzymes.

Examples of the methods for imparting or enhancing L-glutamic acid-producing ability also include, for example, a method for enhancing the expression of an L-glutamic acid secretion gene, such as yhfK gene (WO2005/085419) or ybjL gene (WO2008/133161).

Examples of the methods for imparting or enhancing L-glutamic acid-producing ability also include methods for imparting resistance to organic acid analogues, respiratory inhibitors, or the like, and methods for imparting sensitivity to cell wall synthesis inhibitors. Specific examples of such methods include, for example, the method for imparting monofluoroacetic acid resistance (Japanese Patent Laid-open (Kokai) No. 50-113209), the method for imparting adenine resistance or thymine resistance (Japanese Patent Laid-open (Kokai) No. 57-065198), the method for attenuating urease (Japanese Patent Laid-open (Kokai) No. 52-038088), the method for imparting malonic acid resistance (Japanese Patent Laid-open (Kokai) No. 52-038088), the method for imparting resistance to benzopyrones or naphthoquinones (Japanese Patent Laid-open (Kokai) No. 56-1889), the method for imparting HOQNO resistance (Japanese Patent Laid-open (Kokai) No. 56-140895), the method for imparting α-ketomalonic acid resistance (Japanese Patent Laid-open (Kokai) No. 57-2689), the method for imparting guanidine resistance (Japanese Patent Laid-open (Kokai) No. 56-35981), the method for imparting sensitivity to penicillin (Japanese Patent Laid-open (Kokai) No. 4-88994), and so forth.

Examples of the methods for imparting or enhancing L-glutamic acid-producing ability also include methods for enhancing the expression of yggB gene and a method for introducing a mutant yggB gene having a mutation in the coding region (WO2006/070944). In other words, the L-glutamic acid-producing bacterium may have been modified so that the expression of yggB gene is increased, or may have been modified so as to harbor (have) a mutant yggB gene.

Such modification of a bacterium that the bacterium has a mutant yggB gene can be attained by introducing the mutant yggB gene into the bacterium. Such modification of a bacterium that the bacterium has a mutant yggB gene can also be attained by introducing a mutation into the yggB gene of the bacterium through natural mutation or a treatment with a mutagen.

Examples of the methods for imparting or enhancing L-glutamic acid-producing ability also include methods for modifying a bacterium so that the activity of phosphoketolase is increased (WO2006/016705). Examples of phosphoketolase include D-xylulose-5-phosphate phosphoketolase and fructose-6-phosphate phosphoketolase. Either one of the D-xylulose-5-phosphate phosphoketolase activity and the fructose-6-phosphate phosphoketolase activity may be enhanced, or both may be enhanced. Both the D-xylulose-5-phosphate phosphoketolase activity and the fructose-6-phosphate phosphoketolase activity may also be retained by a single enzyme (i.e. D-xylulose-5-phosphate phosphoketolase/fructose-6-phosphate phosphoketolase).

The genes and proteins used for breeding the L-glutamic acid-producing bacterium may have, for example, the nucleotide sequences and amino acid sequences of known genes and proteins, such as those exemplified above, respectively. Furthermore, the genes and proteins used for breeding the L-glutamic acid-producing bacterium may be conservative variants of known genes and proteins, such as those exemplified above, respectively. The term “Conservative variant” refers to a variant that maintains the original function thereof (such as an activity of enzyme). Specifically, for example, the genes used for breeding the L-glutamic acid-producing bacterium may each be a gene encoding a protein having an amino acid sequence of a known protein, but including substitution, deletion, insertion, or addition of one or several (e.g. from 1 to 50, 1 to 40, or 1 to 30, preferably 1 to 20, more preferably 1 to 10, still more preferably 1 to 5, particularly preferably 1 to 3) amino acid residues at one or several positions, so long as the original function thereof is maintained.

Examples of the L-glutamic acid-producing bacteria include bacteria each having a “specified mutation”.

Examples of the “specified mutation” include the mutations shown in Table 1. The mutations shown in Table 1 consists of 135 mutations of A-1 to A-135, and 92 mutations of B-1 to B-92. Mutations A-1 to A-135 are also referred to as “mutations of Group A”. Mutations B-1 to B-92 are also referred to as “mutations of Group B”.

TABLE 1

| | | Base | Base |
|---|---|---|---|
| | Position | before | after |
| No. | on genome | mutation | mutation |
| A-1 | 78,486 | C | T |
| A-2 | 83,592 | G | A |
| A-3 | 87,955 | C | T |
| A-4 | 90,041 | C | T |
| A-5 | 186,221 | C | T |
| A-6 | 193,010 | C | T |
| A-7 | 196,531 | C | T |
| A-8 | 225,429 | C | T |
| A-9 | 297,920 | G | A |
| A-10 | 320,354 | C | T |
| A-11 | 335,878 | C | T |
| A-12 | 341,763 | C | T |
| A-13 | 346,969 | C | T |
| A-14 | 349,856 | C | T |
| A-15 | 356,232 | C | T |
| A-16 | 357,008 | C | T |
| A-17 | 366,674 | G | A |
| A-18 | 369,871 | G | A |
| A-19 | 377,420 | G | A |
| A-20 | 378,652 | G | A |
| A-21 | 432,252 | C | A |
| A-22 | 439,021 | G | A |
| A-23 | 440,764 | G | A |
| A-24 | 454,682 | G | A |
| A-25 | 458,729 | G | A |
| A-26 | 470,562 | G | A |
| A-27 | 471,288 | G | A |
| A-28 | 472,023 | G | A |
| A-29 | 504,885 | G | A |
| A-30 | 505,785 | G | A |
| A-31 | 514,371 | G | A |
| A-32 | 518,684 | G | A |
| A-33 | 521,126 | G | A |
| A-34 | 524,551 | G | A |
| A-35 | 660,841 | C | T |
| A-36 | 732,121 | C | T |
| A-37 | 787,055 | C | T |
| A-38 | 806,047 | C | T |
| A-39 | 872,482 | G | A |
| A-40 | 878,069 | C | T |
| A-41 | 903,037 | C | T |
| A-42 | 922,802 | C | T |
| A-43 | 948,145 | C | T |
| A-44 | 955,819 | C | T |
| A-45 | 968,915 | C | T |
| A-46 | 973,013 | C | T |
| A-47 | 974,797 | C | T |
| A-48 | 994,815 | C | T |
| A-49 | 1,000,498 | C | T |
| A-50 | 1,019,704 | C | T |
| A-51 | 1,049,052 | C | T |
| A-52 | 1,069,322 | C | T |
| A-53 | 1,070,554 | C | T |
| A-54 | 1,131,016 | C | T |
| A-55 | 1,138,639 | C | T |
| A-56 | 1,162,588 | C | T |
| A-57 | 1,193,273 | C | T |
| A-58 | 1,203,146 | C | T |
| A-59 | 1,222,633 | C | T |
| A-60 | 1,226,969 | G | A |
| A-61 | 1,264,895 | G | A |
| A-62 | 1,268,790 | G | A |
| A-63 | 1,279,676 | G | A |
| A-64 | 1,363,909 | T | C |
| A-65 | 1,387,476 | G | A |
| A-66 | 1,401,171 | G | A |
| A-67 | 1,416,228 | C | T |
| A-68 | 1,420,034 | C | T |
| A-69 | 1,447,494 | C | T |
| A-70 | 1,448,318 | C | T |
| A-71 | 1,448,776 | C | T |
| A-72 | 1,451,922 | C | T |
| A-73 | 1,466,961 | C | T |
| A-74 | 1,503,736 | C | T |
| A-75 | 1,504,207 | C | T |
| A-76 | 1,505,998 | C | T |
| A-77 | 1,507,027 | C | T |
| A-78 | 1,544,310 | C | T |
| A-79 | 1,554,973 | C | T |
| A-80 | 1,558,509 | C | T |
| A-81 | 1,562,459 | C | T |
| A-82 | 1,572,716 | C | T |
| A-83 | 1,594,314 | C | T |
| A-84 | 1,602,545 | C | T |
| A-85 | 1,659,808 | C | T |
| A-86 | 1,682,132 | C | T |
| A-87 | 1,689,863 | C | T |
| A-88 | 1,744,963 | C | T |
| A-89 | 1,784,642 | C | T |
| A-90 | 1,814,866 | C | T |
| A-91 | 1,829,145 | C | T |
| A-92 | 1,852,511 | G | A |
| A-93 | 1,861,170 | G | A |
| A-94 | 1,902,133 | G | A |
| A-95 | 1,916,048 | C | T |
| A-96 | 1,917,434 | C | T |
| A-97 | 1,938,271 | C | T |
| A-98 | 1,949,357 | G | T |
| A-99 | 1,954,368 | C | T |
| A-100 | 1,967,997 | C | T |
| A-101 | 1,975,599 | C | T |
| A-102 | 2,141,466 | C | T |
| A-103 | 2,308,064 | C | T |
| A-104 | 2,310,428 | C | T |
| A-105 | 2,354,420 | C | T |
| A-106 | 2,449,270 | T | C |
| A-107 | 2,449,278 | C | A |
| A-108 | 2,449,291 | G | C |
| A-109 | 2,449,318 | G | A |
| A-110 | 2,496,945 | C | T |
| A-111 | 2,505,022 | C | T |
| A-112 | 2,505,285 | C | T |
| A-113 | 2,525,513 | G | A |
| A-114 | 2,565,856 | C | T |
| A-115 | 2,601,306 | G | A |
| A-116 | 2,615,688 | G | A |
| A-117 | 2,650,740 | G | A |
| A-118 | 2,653,259 | G | A |
| A-119 | 2,663,827 | G | A |
| A-120 | 2,667,322 | G | A |
| A-121 | 2,674,077 | G | A |
| A-122 | 2,679,915 | G | A |
| A-123 | 2,686,979 | G | A |
| A-124 | 2,693,950 | ç | T |
| A-125 | 2,696,737 | C | T |
| A-126 | 2,706,442 | C | T |
| A-127 | 2,709,469 | C | T |
| A-128 | 2,711,214 | C | T |
| A-129 | 2,714,651 | C | T |
| A-130 | 2,721,339 | G | A |
| A-131 | 2,731,030 | G | A |
| A-132 | 2,746,202 | G | A |
| A-133 | 2,805,389 | C | T |
| A-134 | 2,816,733 | G | A |
| A-135 | 2,827,114 | G | A |
| B-1 | 29,724 | G | A |
| B-2 | 92,869 | G | A |
| B-3 | 116,733 | G | A |
| B-4 | 131,184 | G | A |
| B-5 | 156,247 | G | A |
| B-6 | 177,083 | G | A |
| B-7 | 184,379 | G | A |
| B-8 | 212,586 | G | A |
| B-9 | 282,162 | G | A |
| B-10 | 309,483 | G | A |
| B-11 | 376,164 | C | T |
| B-12 | 440,885 | C | T |
| B-13 | 479,120 | G | A |
| B-14 | 722,430 | G | A |
| B-15 | 745,504 | G | A |
| B-16 | 809,993 | G | A |
| B-17 | 859,643 | G | A |
| B-18 | 923,209 | G | A |
| B-19 | 924,973 | G | A |
| B-20 | 998,893 | C | T |
| B-21 | 1,062,144 | C | T |
| B-22 | 1,095,062 | C | T |
| B-23 | 1,102,484 | C | T |
| B-24 | 1,103,812 | C | T |
| B-25 | 1,105,749 | C | T |
| B-26 | 1,107,561 | C | T |
| B-27 | 1,205,722 | C | T |
| B-28 | 1,233,449 | C | T |
| B-29 | 1,242,484 | C | T |
| B-30 | 1,248,388 | C | T |
| B-31 | 1,249,270 | C | T |
| B-32 | 1,291,377 | C | T |
| B-33 | 1,308,597 | C | T |
| B-34 | 1,329,535 | C | T |
| B-35 | 1,367,486 | C | T |
| B-36 | 1,382,065 | C | T |
| B-37 | 1,403,043 | C | T |
| B-38 | 1,433,914 | C | T |
| B-39 | 1,442,447 | C | T |
| B-40 | 1,501,903 | G | A |
| B-41 | 1,504,744 | C | T |
| B-42 | 1,651,403 | G | A |
| B-43 | 1,695,473 | G | A |
| B-44 | 1,779,939 | G | A |
| B-45 | 1,797,452 | G | A |
| B-46 | 1,801,284 | G | A |
| B-47 | 1,816,679 | G | A |
| B-48 | 1,832,252 | G | A |
| B-49 | 1,843,841 | G | A |
| B-50 | 1,868,285 | G | A |
| B-51 | 1,879,922 | G | A |
| B-52 | 1,892,007 | G | A |
| B-53 | 1,916,016 | G | A |
| B-54 | 1,937,604 | G | A |
| B-55 | 1,947,044 | G | A |
| B-56 | 1,948,411 | G | A |
| B-57 | 1,948,649 | G | A |
| B-58 | 1,967,697 | G | A |
| B-59 | 1,974,137 | G | A |
| B-60 | 2,028,284 | C | T |
| B-61 | 2,050,998 | G | A |
| B-62 | 2,052,708 | G | A |
| B-63 | 2,054,372 | G | A |
| B-64 | 2,065,568 | G | A |
| B-65 | 2,067,167 | G | A |
| B-66 | 2,082,577 | G | A |
| B-67 | 2,121,006 | A | G |
| B-68 | 2,149,369 | C | T |
| B-69 | 2,159,680 | G | A |
| B-70 | 2,380,965 | G | A |
| B-71 | 2,477,728 | G | A |
| B-72 | 2,542,800 | G | A |
| B-73 | 2,570,107 | G | A |
| B-74 | 2,647,383 | G | A |
| B-75 | 2,726,248 | C | T |
| B-76 | 2,825,055 | C | T |
| B-77 | 2,837,078 | C | T |
| B-78 | 2,865,322 | C | T |
| B-79 | 2,872,907 | C | T |
| B-80 | 2,880,351 | C | T |
| B-81 | 2,889,394 | C | T |
| B-82 | 2,906,471 | C | T |
| B-83 | 2,927,044 | C | T |
| B-84 | 2,929,963 | C | T |
| B-85 | 2,940,673 | C | T |
| B-86 | 2,946,285 | C | T |
| B-87 | 2,962,909 | C | T |
| B-88 | 2,975,742 | C | T |
| B-89 | 2,987,052 | C | T |
| B-90 | 3,079,560 | C | T |
| B-91 | 3,083,927 | C | T |
| B-92 | 3,090,163 | C | T |

The “specified mutation” may be one or more mutations selected from the mutations shown in Table 1. That is, the L-glutamic acid-producing bacterium may have one or more mutations selected from the mutations shown in Table 1.

The L-glutamic acid-producing bacterium may have, for example, one or more mutations selected from the mutations of Group A. The L-glutamic acid-producing bacterium may have, for example, one or more mutations selected from the mutations of Group B. The L-glutamic acid-producing bacterium may have, for example, one or more mutations selected from the mutations of Group A and one or more mutations selected from the mutations of Group B. The L-glutamic acid-producing bacterium may have, for example, one or more mutations selected from the mutations of Group A, and furthermore may have one or more mutations selected from the mutations of Group B. The L-glutamic acid-producing bacterium may have, for example, one or more mutations selected from the mutations of Group B, and furthermore may have one or more mutations selected from the mutations of Group A. In other words, the “specified mutation” may be, for example, one or more mutations selected from the mutations of Group A, one or more mutations selected from the mutations of Group B, or any combination of one or more mutations selected from the mutations of Group A and one or more mutations selected from the mutations of Group B.

The number of mutations selected from the mutations of Group A in the L-glutamic acid-producing bacterium may be, for example, 1 or more, 5 or more, 10 or more, 20 or more, 30 or more, 40 or more, 50 or more, 60 or more, 70 or more, 80 or more, 90 or more, 100 or more, 110 or more, 120 or more, or 130 or more, or may be 135 or less, 130 or less, 120 or less, 110 or less, 100 or less, 90 or less, 80 or less, 70 or less, 60 or less, 50 or less, 40 or less, 30 or less, 20 or less, 10 or less, or 5 or less, or may be any combination that does not contradict with the above-described ranges. Specifically, the number of mutations selected from the mutations of Group A in the L-glutamic acid-producing bacterium may be, for example, from 1 to 5, from 5 to 10, from 10 to 20, from 20 to 30, from 30 to 40, from 40 to 50, from 50 to 60, from 60 to 70, from 70 to 80, from 80 to 90, from 90 to 100, from 100 to 110, from 110 to 120, from 120 to 130, or from 130 to 135. Specifically, the number of mutations selected from the mutations of Group A in the L-glutamic acid-producing bacterium may be 1 or more, 50 or more, 100 or more, 120 or more, 130 or more, or 135.

The number of mutations selected from the mutations of Group B in the L-glutamic acid-producing bacterium may be, for example, 1 or more, 5 or more, 10 or more, 20 or more, 30 or more, 40 or more, 50 or more, 60 or more, 70 or more, 80 or more, or 90 or more, or 92 or less, 90 or less, 80 or less, 70 or less, 60 or less, 50 or less, 40 or less, 30 or less, 20 or less, 10 or less, or 5 or less, or any combination that does not contradict with the above-described ranges. Specifically, the number of mutations selected from the mutations of Group B in the L-glutamic acid-producing bacterium may be, for example, from 1 to 5, from 5 to 10, from 10 to 20, from 20 to 30, from 30 to 40, from 40 to 50, from 50 to 60, from 60 to 70, from 70 to 80, from 80 to 90, or from 90 to 92. Specifically, the number of mutations selected from the mutations of Group B in the L-glutamic acid-producing bacterium may be 1 or more, 30 or more, 60 or more, 70 or more, 80 or more, or 92.

The L-glutamic acid-producing bacterium may have, for example, 50 or more mutations selected from the mutations of Group A and 30 or more mutations selected from the mutations of Group B. The L-glutamic acid-producing bacterium may have, for example, 100 or more mutations selected from the mutations of Group A and 60 or more mutations selected from the mutations of Group B. The L-glutamic acid-producing bacterium may have, for example, 120 or more mutations selected from the mutations of Group A and 80 or more mutations selected from the mutations of Group B. The L-glutamic acid-producing bacterium may have, for example, 135 mutations selected from the mutations of Group A and 92 mutations selected from the mutations of Group B.

The L-glutamic acid-producing bacterium having the “specified mutation” may be particularly a coryneform bacterium. The L-glutamic acid-producing bacterium having the “specified mutation” may be further particularly a bacterium of the genus Corynebacterium. The L-glutamic acid-producing bacterium having the “specified mutation” may be further particularly Corynebacterium casei. The L-glutamic acid-producing bacterium having the “specified mutation” may be further particularly a modified strain derived from Corynebacterium casei JCM 12072.

Specific examples of the L-glutamic acid-producing bacterium having the “specified mutation” may include Corynebacterium casei RUN5-2-96 strain (NITE BP-03688). RUN5-2-96 strain is also referred to as “AJ111891 strain”. RUN5-2-96 strain is a modified strain derived from Corynebacterium casei JCM 12072 and has all 135 mutations of Group A. RUN5-2-96 strain has been originally deposited as an international deposit with the Patent Microorganisms Depositary, National Institute of Technology and Evaluation (independent organization) (NITE NPMD, postal code: 292-0818, address: Room 122, 2-5-8, Kazusakamatari, Kisarazu-shi, Chiba, Japan) on Jul. 7, 2022, and Accession No. NITE BP-03688 is assigned. RUN5-2-96 strain is available from, for example, NITE NPMD.

In Table 1, “Position on genome” represents the position of each mutation on a base sequence registered as ACCESSION No. NZ_CP004350.1 on the NCBI (National Center for Biotechnology Information; https://www.ncbi.nlm.nih.gov/). Hereinafter, this base sequence is also referred to as “base sequence of NZ_CP004350.1”. The base sequence of NZ_CP004350.1 is the base sequence of the genome of Corynebacterium casei JCM 12072 (LMG S-19264), and is published with annotations. The base sequence of NZ_CP004350.1 and the annotation of each position on the genome can be obtained from, for example, NCBI.

Each mutation shown in Table 1 is to be interpreted as the mutation at the position corresponding to the position of each mutation shown in Table 1, on the genome of each bacterium. For example, mutation A-1 is to be interpreted as a mutation at the position corresponding to the 78,486-th position of the base sequence of NZ_CP004350.1, on the genome of each bacterium. The position of each mutation shown in Table 1 is denoted for convenience in order to identify each mutation, and it is not necessary to show any absolute position on the genome of each bacterium. That is, the position of each mutation shown in Table 1 indicates a relative position based on the base sequence of NZ_CP004350.1, and the absolute position may be shifted upstream or downstream due to deletion, insertion, or the like of nucleic acid residue(s). For example, when one nucleic acid residue is deleted or inserted at a position at the 5′ terminal side relative to the X-th position (X is a positive integer) in the base sequence of NZ_CP004350.1, the original X-th position corresponds to the X−1-th position or the X+1-th position, but the mutation at the original X-th position is regarded as “mutation at the position corresponding to the X-th position of the base sequence of NZ_CP004350.1”. The base before mutation shown in Table 1 is denoted for convenience in order to identify each mutation and is not needed to be preserved on the genome of a bacterium before modification. That is, when the genome of a bacterium before modification has no base sequence of NZ_CP004350.1, no base before mutation shown in Table 1 may not be preserved. That is, the phrase “introduction of mutation into bacterium” with respect to each mutation shown in Table 1 means that the base (this is any base other than the base after mutation) at the position of each mutation shown in Table 1 on the genome of a bacterium before modification is modified to a base after mutation shown in Table 1. For example, the phrase “introduction of mutation A-1 into bacterium” means that the base (this is C, G, or A) at the position corresponding to the 78,486-th position of the base sequence of NZ_CP004350.1, on the genome of a bacterium before modification, is modified to T.

Which position on the genome of each bacterium is “position corresponding to the position of each mutation shown in Table 1” can be determined by carrying out alignment between the base sequence of the genome of each bacterium and the base sequence of NZ_CP004350.1. Such alignment can be carried out by using, for example, known gene analysis software. Examples of such gene analysis software include DNASIS manufactured by Hitachi Solutions, Ltd. and GENETYX manufactured by GENETYX CORPORATION (Elizabeth C. Tyler et al., Computers and Biomedical Research, 24 (1), 72-96, 1991; Barton G J et al., Journal of molecular biology, 198 (2), 327-37. 1987).

The phrase “bacterium having mutation” with respect to each mutation shown in Table 1 means that the base at the position of such a mutation on the genome of such a bacterium is the base after mutation shown in Table 1, and does not necessarily mean that such a bacterium is obtained by introduction of such a mutation. In other words, “bacterium having mutation” with respect to each mutation shown in Table 1 may be naturally a bacterium where the base at the position of such a mutation is the base after mutation shown in Table 1, or may be naturally a bacterium obtained by modifying a bacterium where the base at the position of such a mutation is not the base after mutation shown in Table 1. For example, the phrase “bacterium having mutation A-1” means that the position (the position corresponding to the 78,486-th position of the base sequence of NZ_CP004350.1) of mutation A-1 on the genome of such a bacterium is T, and does not necessarily mean that such a bacterium is obtained by introduction of mutation A-1. In other words, for example, “bacterium having mutation A-1” may be naturally a bacterium where the base at the position of mutation A-1 (the position corresponding to the 78,486-th position of the base sequence of NZ_CP004350.1) on the genome is T, or may be naturally a bacterium obtained by modifying a bacterium where the base at the position of mutation A-1 on the genome is not T. The L-glutamic acid-producing bacterium having “specified mutation” may be particularly one obtained by introduction of one portion or the whole of “specified mutation”.

The L-glutamic acid-producing bacterium having “specified mutation” can be obtained by, for example, introduction of “specified mutation” into a bacterium having no “specified mutation”. The L-glutamic acid-producing bacterium having “specified mutation” can be obtained by, for example, introducing the remaining portion of “specified mutation” to a bacterium having one portion of “specified mutation”.

The introduction of mutations can be performed by, for example, a known procedure. For example, an objective mutation can be introduced to an objective position on a genome by site-directed mutagenesis. Examples of the site-directed mutagenesis may include methods with PCR (Higuchi, R., 61, in PCR technology, Erlich, H. A. Eds., Stockton press (1989); Carter, P., Meth. in Enzymol., 154, 382 (1987)) and methods with phage (Kramer, W. and Fritz, H. J., Meth. in Enzymol., 154, 350 (1987); Kunkel, T. A. et al., Meth. in Enzymol., 154, 367 (1987)).

Each mutation shown in Table 1 may be a mutation which enhances an L-glutamic acid-producing ability of a bacterium. Each mutation shown in Table 1 may be particularly a mutation which enhances an L-glutamic acid-producing ability of a bacterium as compared with a case where the base at the position of each mutation is the base before mutation shown in Table 1.

Each mutation shown in Table 1 may be, for example, a mutation of a gene (which here means its coding region), a mutation in an expression-regulating region of a gene such as a promoter, or a mutation in an intergenic region. Which region (for example, a gene, an expression-regulating region of a gene, or an intergenic region) each mutation shown in Table 1 is located in can be confirmed by, for example, referring to the annotations at the position of each mutation and the base sequence of NZ_CP004350.1.

When each mutation shown in Table 1 is a mutation of a gene, each mutation may cause, for example, the change (for example, increase or decrease) in expression of such a gene. When each mutation shown in Table 1 is a mutation of a gene, each mutation may cause, for example, the change (for example, increase or decrease) in the activity of the protein encoded by such a gene.

When each mutation shown in Table 1 is a mutation of an expression-regulating region of a gene, each mutation may cause, for example, the change (for example, increase or decrease) in expression of such a gene.

The change (for example, increase or decrease) in expression of a gene may cause, for example, the change (for example, increase or decrease) in the activity of the protein encoded by such a gene.

The L-glutamic acid-producing bacterium having “specified mutation” may or may not have other modifications than “specified mutation” as long as it has an L-glutamic acid-producing ability. Examples of other modifications than “specified mutation” include known modifications for imparting or enhancing an L-glutamic acid-producing ability. Examples of other modifications than “specified mutation” include modifications not selected as “specified mutation” shown in Table 1. The L-glutamic acid-producing bacterium having “specified mutation” may have, for example, an L-glutamic acid-producing ability depending on “specified mutation”, or an L-glutamic acid-producing ability depending on a combination of “specified mutation” and other modifications than “specified mutation”.

Specific examples of the L-glutamic acid-producing bacterium include Corynebacterium casei A-013 (NITE BP-03806). A-013 strain is also referred to as the AJ120306 strain. Corynebacterium casei A-013 strain is a modified strain derived from Corynebacterium casei JCM 12072. A-013 strain has been originally deposited as an international deposit with the Patent Microorganism Depositary, National Institute of Technology and Evaluation (Independent Administrative Institution) (NITE NPMD, postal code: 292-0818, address: Room 122, 2-5-8, Kazusakamatari, Kisarazu-shi, Chiba, Japan) on Jan. 25, 2023, and Accession No. NITE BP-03806 is assigned. A-013 strain is available from, for example, NITE NPMD.

An L-glutamic acid-producing bacterium such as the L-glutamic acid-producing bacterium having “specified mutation” may have, for example, a remarkably higher L-glutamic acid-producing ability than that of Corynebacterium casei JCM 12072. The phrase “remarkably higher L-glutamic acid-producing ability than that of Corynebacterium casei JCM 12072” may mean, for example, an ability to produce and accumulate L-glutamic acid in an amount twice or more, 3 times or more, 5 times or more, or 7 times or more that of JCM 12072, in a medium, in culture under appropriate culture conditions. The L-glutamic acid-producing bacterium such as the L-glutamic acid-producing bacterium having “specified mutation” may have, for example, an L-glutamic acid-producing ability equivalent to or more than that of Corynebacterium casei RUN5-2-96 strain (NITE BP-03688) or Corynebacterium casei A-013 strain (NITE BP-03806). The phrase “L-glutamic acid-producing ability equivalent to or more than that of a Corynebacterium casei RUN5-2-96 strain (NITE BP-03688) or Corynebacterium casei A-013 strain (NITE BP-03806)” may mean, for example, an ability to produce L-glutamic acid in an amount of 80% or more, 90% or more, 95% or more, or 100% or more relative to RUN5-2-96 strain or Corynebacterium casei A-013 strain (NITE BP-03806), in a medium, in culture under appropriate culture conditions. Examples of appropriate culture conditions include the culture condition for measuring the amount of L-glutamic acid produced, as mentioned in Example 1 (2) described later (i.e. the condition of shaking and culture at 30° C. for 48 hours in a 500 μL evaluation medium (Table 4) contained in a 96 deep well plate), or the “oxygen limitation only” condition or the “temperature elevation×oxygen limitation” condition as mentioned in Examples 2 to 5 later.

The base sequence of the genome of an L-glutamic acid-producing bacterium such as the L-glutamic acid-producing bacterium having “specified mutation” may be 95% or more, 96% or more, 97% or more, 98% or more, 99% or more, 99.5% or more, 99.7% or more, 99.9% or more, 99.95% or more, 99.97% or more, or 99.99% or more identical to that of, for example, Corynebacterium casei JCM 12072, Corynebacterium casei RUN5-2-96 strain (NITE BP-03688), or Corynebacterium casei A-013 strain (NITE BP-03806). The term “identity” between base sequences means the identity between base sequences calculated with Scoring Parameters of default settings (Match/Mismatch scores=1, −2; Gap Costs=Linear), by blastn.

### <1-1> Methods for Increasing the Activity of a Protein

Hereinafter, the methods for increasing the activity of a protein (for example, enzymes such as L-glutamic acid biosynthesis enzymes) will be explained.

The expression “the activity of a protein is increased” means that the activity of the protein is increased as compared with that of a non-modified strain. The expression “the activity of a protein is increased” means that, specifically, the activity of the protein per cell is increased as compared with that of a non-modified strain. The term “non-modified strain” used herein refers to a control strain that has not been modified so that the activity of an objective protein is increased. Examples of the non-modified strain include a wild-type strain and a parent strain. Examples of the non-modified strain include, specifically, a type strain of Corynebacterium casei. Furthermore, examples of the non-modified strain also include, specifically, Corynebacterium casei JCM 12072. That is, in an embodiment, the activity of a protein may be increased as compared with that of a type strain of Corynebacterium casei. In another embodiment, the activity of a protein may be increased as compared with that of Corynebacterium casei JCM 12072. The state that “the activity of a protein is increased” may also mean “the activity of a protein is enhanced”. Specifically, the expression “the activity of a protein is increased” means that the number of molecules of the protein per cell is increased, and/or the function of each molecule of the protein is increased as compared with those of a non-modified strain. That is, the term “activity” in the expression “the activity of a protein is increased” is not limited to the catalytic activity of the protein, but may also mean the transcription level of a gene (i.e. amount of mRNA) encoding the protein, or the translation level of the protein (i.e. amount of the protein). The expression “the number of molecules of the protein per cell” may mean the average number of molecules of the protein per cell. Furthermore, the state that “the activity of a protein is increased” includes not only a state that the activity of an objective protein is increased in a strain inherently having the activity of the objective protein, but also a state that the activity of an objective protein is imparted to a strain not inherently having the activity of the objective protein. Furthermore, so long as the activity of the protein is eventually increased, the activity of an objective protein inherently contained in a host may be attenuated and/or eliminated, and then an appropriate type of the objective protein may be imparted to the host.

The degree of the increase in the activity of a protein is not particularly limited, so long as the activity of the protein is increased as compared with a non-modified strain. The activity of the protein may be increased to, for example, 1.5 times or more, 2 times or more, or 3 times or more of that of the non-modified strain. Furthermore, when the non-modified strain does not have the activity of the objective protein, it is sufficient that the protein is produced as a result of introduction of the gene encoding the protein, and for example, the protein may be produced to such an extent that the activity thereof can be measured.

A modification for increasing the activity of a protein is attained by, for example, increasing the expression of a gene encoding the protein. The expression “expression of a gene is increased” means that the expression level of the gene is increased as compared with that of a non-modified strain such as a wild-type strain and a parent strain. Specifically, the expression “expression of a gene is increased” may mean that the expression level of the gene per cell is increased as compared with that of a non-modified strain. The expression “expression level of a gene per cell” may mean the average of the expression level of the gene per cell. More specifically, the expression “expression of a gene is increased” may mean that the transcription level of the gene (i.e. amount of mRNA) is increased, and/or the translation level of the gene (i.e. amount of the protein expressed from the gene) is increased. The state that the “expression of a gene is increased” may also be referred to as the “expression of a gene is enhanced”. The expression of the gene may be increased to, for example, 1.5 times or more, 2 times or more, or 3 times or more of that of the non-modified strain. Furthermore, the state that the “expression of a gene is increased” includes not only a state that the expression level of an objective gene is increased in a strain that inherently expresses the objective gene, but also a state that the gene is introduced into a strain that does not inherently express the objective gene, and expressed therein. That is, the phrase “expression of a gene is increased” may also mean, for example, that an objective gene is introduced into a strain that does not possess the gene, and is expressed therein.

The expression of a gene can be increased by, for example, increasing the copy number of the gene.

The copy number of the gene can be increased by introducing the gene into the chromosome of a host. A gene can be introduced into a chromosome by, for example, using homologous recombination (Miller, J. H., Experiments in Molecular Genetics, 1972, Cold Spring Harbor Laboratory). Examples of the gene transfer methods utilizing homologous recombination include, for example, a method using a linear DNA such as Red-driven integration (Datsenko, K. A., and Wanner, B. L., Proc. Natl. Acad. Sci. USA, 97:6640-6645 (2000)), a method using a plasmid containing a temperature sensitive replication origin, a method using a plasmid capable of conjugative transfer, a method using a suicide vector not having a replication origin that functions in a host, and a transduction method using a phage. Specifically, the gene can be introduced into the chromosome of the host by transforming the host with recombinant DNA containing the target gene and causing homologous recombination between the target site on the chromosome of the host and the recombinant DNA. The structure of the recombinant DNA used for homologous recombination is not particularly limited, so long as homologous recombination can occur in a desired manner. For example, transformation may be performed with linear DNA containing the target gene and having nucleotide sequences homologous to upstream and downstream regions of the chromosomal target site at each end of the gene, respectively. Homologous recombination occurring both upstream and downstream of the target site allows replacement of the target site with the gene. The recombinant DNA used for homologous recombination may comprise a marker gene for selecting transformants. Only one copy, or two or more copies of a gene may be introduced. For example, by performing homologous recombination using a sequence that is present in multiple copies on a chromosome as a target, multiple copies of a gene can be introduced into the chromosome. Examples of such a sequence that is present in multiple copies on a chromosome include repetitive DNA, and inverted repeats located at both ends of a transposon. Furthermore, homologous recombination may be performed by using an appropriate sequence on a chromosome such as a gene unnecessary for production of L-glutamic acid as a target. Furthermore, a gene can also be randomly introduced into a chromosome by using a transposon or Mini-Mu (Japanese Patent Laid-open (Kokai) No. 2-109985, U.S. Pat. No. 5,882,888, and EP805867B1). Such chromosome modification methods utilizing homologous recombination are not limited to introducing a gene, and may be used for any modification of the chromosome, such as a modification of regulatory sequences.

Introduction of a target gene into a chromosome can be confirmed by Southern hybridization using a probe having a sequence complementary to the whole gene or a part thereof, PCR using primers prepared on the basis of the sequence of the gene, or the like.

Furthermore, the copy number of a gene can also be increased by introducing a vector containing the gene into a host. For example, the copy number of a target gene can be increased by ligating a DNA fragment containing a target gene with a vector that functions in a host to construct an expression vector of the gene, and transforming the host with the expression vector. The DNA fragment containing the target gene can be obtained by, for example, PCR using the genomic DNA of a microorganism having the target gene as the template. As the vector, a vector autonomously replicable in the cell of the host can be used. The vector is preferably a multicopy vector. Furthermore, the vector preferably has a marker such as an antibiotic resistance gene for selection of transformant. Furthermore, the vector may have a promoter and/or terminator for expressing the introduced gene. The vector may be, for example, a vector derived from a bacterial plasmid, a vector derived from a yeast plasmid, a vector derived from a bacteriophage, cosmid, phagemid, or the like. Specific examples of vector autonomously replicable in Coryneform bacteria include, for example, pHM1519 (Agric. Biol. Chem., 48, 2901-2903 (1984)); pAM330 (Agric. Biol. Chem., 48, 2901-2903 (1984)); plasmids comprising drug resistance genes that are improved variants thereof; pCRY30 (Japanese Patent Laid-open (Kokai) No. 3-210184); pCRY21, pCRY2KE, pCRY2KX, pCRY31, pCRY3KE, and pCRY3KX (Japanese Patent Laid-open (Kokai) No. 2-72876, U.S. Pat. No. 5,185,262); pCRY2 and pCRY3 (Japanese Patent Laid-open (Kokai) No. 1-191686); pAJ655, pAJ611, and pAJ1844 (Japanese Patent Laid-open (Kokai) No. 58-192900); pCG1 (Japanese Patent Laid-open (Kokai) No. 57-134500); pCG2 (Japanese Patent Laid-open (Kokai) No. 58-35197); pCG4 and pCG11 (Japanese Patent Laid-open (Kokai) No. 57-183799); pVK7 (Japanese Patent Laid-open (Kokai) No. 10-215883); pVK9 (US2006-0141588); pVC7 (Japanese Patent Laid-open (Kokai) No. 9-070291); and pVS7 (WO2013/069634). Furthermore, specific examples of the vectors autonomously replicable in coryneform bacteria include, for example, variants of pVC7, such as pVC7H2 (WO2018/179834).

When a gene is introduced, it is sufficient that the gene is expressibly harbored by a host. Specifically, it is sufficient that the gene is harbored by a host so that it is expressed under control by a promoter sequence that functions in the host. The promoter is not particularly limited so long as it functions in the host. The expression “promoter that functions in a host” refers to a promoter that shows a promoter activity in the host. The promoter may be a promoter derived from the host, or a heterologous promoter. The promoter may be the native promoter of the gene to be introduced, or a promoter of another gene. As the promoter, for example, such a stronger promoter as mentioned later may also be used.

A terminator for termination of gene transcription may be located downstream of the gene. The terminator is not particularly limited so long as it functions in the host. The terminator may be a terminator derived from the host, or a heterologous terminator. The terminator may be the native terminator of the gene to be introduced, or a terminator of another gene.

Vectors, promoters, and terminators that can be used in various microorganisms are disclosed in detail, for example, in “Microbiology Basic Course 8: Genetic Engineering, Kyoritsu Shuppan, 1987”, and these elements can be used.

Furthermore, when two or more genes are introduced, it is sufficient that the genes each are expressibly harbored by the host. For example, all the genes may be harbored by a single expression vector or on the chromosome. Furthermore, the genes may be separately harbored by two or more expression vectors, or separately harbored by a single or two or more expression vectors and on the chromosome. An operon constituted by two or more genes may also be introduced. The cases of “introducing two or more genes” include, for example, cases of introducing respective genes encoding two or more kinds of proteins (such as enzymes), cases of introducing respective genes encoding two or more subunits constituting a single protein complex (such as enzyme complex), and a combination thereof.

The gene to be introduced is not particularly limited so long as it encodes a protein that functions in the host. The gene to be introduced may be a gene derived from the host, or may be a heterologous gene. The gene to be introduced can be obtained by, for example, PCR using primers designed on the basis of the nucleotide sequence of the gene, and using the genomic DNA of an organism having the gene, a plasmid carrying the gene, or the like as a template. The gene to be introduced may also be totally synthesized, for example, on the basis of the nucleotide sequence of the gene (Gene, 60(1), 115-127 (1987)). The obtained gene can be used as it is, or after being modified as required. A gene can be modified by a known procedure. For example, an objective mutation can be introduced into an objective site of DNA by site-directed mutagenesis. That is, the coding region of a gene can be modified by the site-directed mutagenesis so that a specific site of the encoded protein includes substitution, deletion, insertion, or addition of amino acid residues. Examples of the site-directed mutagenesis may include the method utilizing PCR (Higuchi, R., 61, in PCR Technology, Erlich, H. A. Eds., Stockton Press (1989); Carter, P., Meth. in Enzymol., 154, 382 (1987)), and the method utilizing phage (Kramer, W. and Frits, H. J., Meth. in Enzymol., 154, 350 (1987); Kunkel, T. A. et al., Meth. in Enzymol., 154, 367 (1987)). Alternatively, a variant of the gene may also be synthesized in its entirety.

Incidentally, when a protein functions as a complex consisting of a plurality of subunits, all or a part of the plurality of subunits may be modified, so long as the activity of the protein is eventually increased. That is, for example, when the activity of a protein is increased by increasing the expression of a gene, the expression of all or a part of the plurality of genes that encode the subunits may be enhanced. It is usually preferable to enhance the expression of all of the plurality of genes encoding the subunits. Furthermore, the subunits constituting the complex may be derived from a single kind of organism or two or more kinds of organisms, so long as the complex has a function of the objective protein. That is, for example, genes of the same organism encoding a plurality of subunits may be introduced into a host, or genes of different organisms encoding a plurality of subunits may be introduced into a host.

Furthermore, the expression of a gene can be increased by improving the transcription efficiency of the gene. In addition, the expression of a gene can also be increased by improving the translation efficiency of the gene. The transcription efficiency of the gene and the translation efficiency of the gene can be improved by, for example, modifying an expression control sequence of the gene. The term “expression control sequence” collectively refers to sites that affect the expression of a gene. Examples of the expression control sequences include, for example, promoter, Shine-Dalgarno (SD) sequence (also referred to as ribosome binding site (RBS)), and spacer region between RBS and the start codon. Expression control sequences can be identified by using a promoter search vector or gene analysis software such as GENETYX. These expression control sequences can be modified by, for example, a method using a temperature sensitive vector, or the Red-driven integration method (WO2005/010175).

The transcription efficiency of the gene can be improved by, for example, replacing a promoter of the gene on a chromosome with a stronger promoter. The term “stronger promoter” refers to a promoter providing an improved transcription of a gene compared with an inherently existing wild-type promoter of the gene. Examples of the stronger promoters usable in coryneform bacteria include, for example, the artificially modified P54-6 promoter (Appl. Microbiol. Biotechnol., 53, 674-679 (2000)), pta, aceA, aceB, adh, and amyE promoters inducible in coryneform bacteria with acetic acid, ethanol, pyruvic acid, or the like, cspB, SOD, and tuf (EF-Tu) promoters, which are potent promoters capable of providing a large expression level in coryneform bacteria (Journal of Biotechnology, 104 (2003) 311-323; Appl. Environ. Microbiol., 2005 December; 71 (12):8587-96), P2 promoter (US2018-0334693A), P3 promoter (US2018-0334693A), F1 promoter (WO2018/179834), lac promoter, tac promoter, and trc promoter. Furthermore, as the stronger promoter, a highly-active type of an existing promoter may also be obtained by using various reporter genes. For example, by making the −35 and −10 regions in a promoter region closer to the consensus sequence, the activity of the promoter can be enhanced (WO00/18935). Examples of the highly active-type promoters include various tac-like promoters (Katashkina J I et al., Russian Federation Patent Application No. 2006134574) and pnlp8 promoter (WO2010/027045). Methods for evaluating the strength of promoters and examples of strong promoters are described in the paper of Goldstein et al. (Prokaryotic Promoters in Biotechnology, Biotechnol. Annu. Rev., 1, 105-128 (1995)), and so forth.

The translation efficiency of the gene can be improved by, for example, replacing the Shine-Dalgarno (SD) sequence (also referred to as ribosome binding site (RBS)) of the gene on a chromosome with a stronger SD sequence. The term “stronger SD sequence” means a SD sequence that provides an improved translation of mRNA compared with the inherently existing wild-type SD sequence of the gene. Examples of stronger SD sequences include, for example, RBS of the gene 10 derived from phage T7 (Olins P. O. et al, Gene, 1988, 73, 227-235). Furthermore, it is known that substitution, insertion, or deletion of several nucleotides in a spacer region between RBS and the start codon, especially in a sequence immediately upstream of the start codon (5′-UTR), significantly affects the stability and translation efficiency of mRNA, and hence, the translation efficiency of a gene can also be improved by modifying them.

The translation efficiency of the gene can also be improved by, for example, modifying codons. For example, the translation efficiency of the gene can be improved by replacing a rare codon present in the gene with a synonymous codon more frequently used. That is, the gene to be introduced may be modified, for example, so as to contain optimal codons according to the frequencies of codons observed in a host to be used. Codons can be replaced by, for example, site-directed mutagenesis for introducing an objective mutation into an objective site of DNA. Alternatively, a gene fragment in which objective codons are replaced may be totally synthesized. Frequencies of codons in various organisms are disclosed in the “Codon Usage Database” (http://www.kazusa.or.jp/codon; Nakamura, Y. et al, Nucl. Acids Res., 28, 292 (2000)).

Furthermore, the expression of the gene can also be increased by amplifying a regulator that increases the expression of the gene, or deleting or attenuating a regulator that reduces the expression of the gene.

Such methods for increasing the gene expression as mentioned above may be used independently or in an arbitrary combination.

Furthermore, the modification that increases the activity of a protein can also be attained by, for example, enhancing the specific activity of a protein. Enhancing the specific activity also includes desensitization to feedback inhibition. A protein showing an enhanced specific activity can be obtained by, for example, searching various organisms. Furthermore, a highly-active type of an existing protein may also be obtained by introducing a mutation into the existing protein. The mutation to be introduced may be, for example, substitution, deletion, insertion, or addition of one or several amino acid residues at one or several positions of the protein. The mutation can be introduced by, for example, such site-directed mutagenesis as mentioned above. The mutation may also be introduced by, for example, a mutagenesis treatment. Examples of the mutagenesis treatments include irradiation of X-ray, irradiation of ultraviolet, and a treatment with a mutation agent such as N-methyl-N′-nitro-N-nitrosoguanidine (MNNG), ethyl methanesulfonate (EMS), and methyl methanesulfonate (MMS). Furthermore, a random mutation may be induced by directly treating DNA in vitro with hydroxylamine. Enhancing the specific activity may be independently used, or may be used in an arbitrary combination with such methods for enhancing gene expression as mentioned above.

The method for the transformation is not particularly limited, and conventionally known methods can be used. Examples of the methods for the transformation include protoplast method (Gene, 39, 281-286 (1985)), electroporation (Bio/Technology, 7, 1067-1070 (1989)), and electric pulse method (Japanese Patent Laid-open (Kokai) No. 2-207791).

An increase in the activity of a protein can be confirmed by measuring the activity of the protein.

An increase in the activity of a protein can also be confirmed by confirming an increase in the expression of a gene encoding the protein. An increase in the expression of a gene can be confirmed by confirming an increase in the transcription level of the gene, or by confirming an increase in the amount of a protein expressed from the gene.

An increase of the transcription level of a gene can be confirmed by comparing the amount of mRNA transcribed from the gene with that of a non-modified strain such as a wild-type strain or a parent strain. Examples of the methods for evaluating the amount of mRNA include Northern hybridization, RT-PCR, microarray, RNA-seq, and so forth (Sambrook, J., et al., Molecular Cloning: A Laboratory Manual/Third Edition, Cold Spring Harbor Laboratory Press, Cold Spring Harbor (USA), 2001). The amount of mRNA (for example, number of molecules per cell) may be increased to, for example, 1.5 times or more, 2 times or more, or 3 times or more of that of a non-modified strain.

An increase in the amount of a protein can be confirmed by Western blotting using antibodies (Molecular Cloning, Cold Spring Harbor Laboratory Press, Cold Spring Harbor (USA), 2001). The amount of the protein (for example, the number of molecules per cell) may be increased to, for example, 1.5 times or more, 2 times or more, or 3 times or more of that of a non-modified strain.

The aforementioned methods for increasing the activity of a protein can be used for enhancing the activities of arbitrary proteins and enhancing the expression of arbitrary genes.

### <1-2> Method for Reducing the Activity of a Protein

Hereinafter, the methods for reducing the activity of a protein (for example, enzymes such as enzymes that catalyse reactions branching from the L-glutamic acid biosynthetic pathway to generate compounds other than L-glutamic acid) will be explained.

The expression “the activity of a protein is reduced” means that the activity of the protein is reduced as compared with that of a non-modified strain. The expression “the activity of a protein is reduced” means that, specifically, the activity of the protein per cell is reduced as compared with that of a non-modified strain. The term “non-modified strain” used herein refers to a control strain that has not been modified so that the activity of an objective protein is reduced. Examples of the non-modified strain include a wild-type strain and parent strain. Examples of the non-modified strain include, specifically, a type strain of Corynebacterium casei. Furthermore, examples of the non-modified strain also include, specifically, Corynebacterium casei JCM 12072. That is, in an embodiment, the activity of a protein may be reduced as compared with that of a type strain of Corynebacterium casei. In another embodiment, the activity of a protein may be reduced as compared with that of Corynebacterium casei JCM 12072. The state that “the activity of a protein is reduced” also includes a state that the activity of the protein has completely disappeared. Specifically, the expression “the activity of a protein is reduced” means that the number of molecules of the protein per cell is reduced, and/or the function of the protein per molecule is reduced as compared with those of a non-modified strain. That is, the term “activity” in the expression “the activity of a protein is reduced” is not limited to the catalytic activity of the protein, but may also mean the transcription level of a gene (i.e. amount of mRNA) encoding the protein, or the translation level of the protein (i.e. amount of the protein). The expression “the number of molecules of the protein per cell” may mean the average number of molecules of the protein per cell. The state that “the number of molecules of the protein per cell is reduced” also includes a state that the protein does not exist at all. The state that “the function of each molecule of the protein is reduced” also includes a state that the function of each protein molecule has completely disappeared. The degree of the reduction in the activity of a protein is not particularly limited, so long as the activity is reduced as compared with that of a non-modified strain. The activity of a protein may be reduced to, for example, 50% or less, 20% or less, 10% or less, 5% or less, or 0% of that of a non-modified strain.

A modification for reducing the activity of a protein can be attained by, for example, reducing the expression of a gene encoding the protein. The expression “expression of a gene is reduced” means that the expression of the gene per cell is reduced as compared with that of a non-modified strain such as a wild-type strain and parent strain. Specifically, the expression “expression of a gene is reduced” may mean that the expression level of the gene per cell is reduced as compared with that of a non-modified strain. The expression “expression level of a gene per cell” may mean the average of the expression level of the gene per cell. The expression “expression of a gene is reduced” may specifically mean that the transcription level of the gene (i.e. amount of mRNA) is reduced, and/or the translation level of the gene (i.e. amount of the protein expressed from the gene) is reduced. The state that “expression of a gene is reduced” also includes a state that the gene is not expressed at all. The state that “expression of a gene is reduced” is also referred to as “expression of a gene is attenuated”. The expression of a gene may be reduced to, for example, 50% or less, 20% or less, 10% or less, 5% or less, or 0% of that of a non-modified strain.

The reduction in gene expression may be due to, for example, a reduction in the transcription efficiency, a reduction in the translation efficiency, or a combination thereof. The expression of a gene can be reduced by modifying an expression control sequence of the gene such as a promoter, Shine-Dalgarno (SD) sequence (also referred to as ribosome-binding site (RBS)), and spacer region between RBS and the start codon of the gene. When an expression control sequence is modified, preferably one or more nucleotides, more preferably two or more nucleotides, particularly preferably three or more nucleotides, of the expression control sequence are modified. A decrease in transcriptional efficiency of the gene can be attained by, for example, replacing the promoter of a chromosomal gene with a weaker promoter. The term “weaker promoter” refers to a promoter that results in weaker transcription of the gene as compared to the originally existing wild-type promoter. Examples of the weaker promoters include inducible promoters. That is, inducible promoters can function as weaker promoters under non-induced conditions (for example, in the absence of an inducer). Further examples of the weaker promoters include the P4 promoter (US2018-0334693A) and the P8 promoter (US2018-0334693A). Furthermore, a part or the whole of an expression control sequence may be deleted. The expression of a gene can also be reduced by, for example, manipulating a factor responsible for expression control. Examples of the factor responsible for expression control include small molecules (such as inducers and inhibitors), proteins (such as transcription factors), nucleic acids (such as siRNA), and so forth that are responsible for transcription or translation control. Furthermore, the expression of a gene can also be reduced by, for example, introducing a mutation that reduces the expression of the gene into the coding region of the gene. For example, the expression of a gene can be reduced by replacing a codon in the coding region of the gene with a synonymous codon used less frequently in a host. Furthermore, for example, the gene expression may be reduced due to disruption of a gene as described later.

The modification for reducing the activity of a protein can also be attained by, for example, disrupting a gene encoding the protein. The expression “gene is disrupted” means that the gene is modified so that the protein that can normally function is not produced. The state that “protein that normally functions is not produced” includes a state that the protein is not produced at all from the gene, and a state that the protein of which the function (such as activity or property) per molecule is reduced or eliminated is produced from the gene.

Disruption of a gene can be attained by, for example, deleting (removing) the gene on a chromosome. The term “gene deletion” refers to the deletion of a part or the whole of the coding region of a gene. Furthermore, the whole of the gene including sequences upstream and downstream from the gene on a chromosome may be deleted. The sequences upstream and downstream from the coding region of a gene may include, for example, regulatory sequences of the gene. The region to be deleted may be any region such as an N-terminus region (region encoding the N-terminus of the protein), an internal region, or a C-terminus region (region encoding the C-terminus of the protein), so long as the activity of the protein can be reduced. Generally, deleting a longer region more reliably inactivates the gene. The deleted region may be, for example, a region accounting for 10% or more, 20% or more, 30% or more, 40% or more, 50% or more, 60% or more, 70% or more, 80% or more, 90% or more, or 95% or more of the total length of the coding region of the gene. Furthermore, it is preferable that reading frames of the sequences upstream and downstream from the deleted region are not the same. The mismatch of reading frames may cause frameshift downstream of the deleted region.

Disruption of a gene can also be attained by, for example, introducing an amino acid substitution (missense mutation), a stop codon (nonsense mutation), a frame shift mutation which adds or deletes one or two nucleotide residues, or the like into the coding region of the gene on a chromosome (Journal of Biological Chemistry, 272:8611-8617 (1997); Proceedings of the National Academy of Sciences, USA, 95 5511-5515 (1998); Journal of Biological Chemistry, 26 116, 20833-20839 (1991)).

Disruption of a gene can also be attained by, for example, inserting another sequence into a coding region of the gene on a chromosome. The insertion site may be in any region of the gene, and insertion of a longer nucleotide sequence can usually more surely inactivate the gene. It is preferable that reading frames of the sequences upstream and downstream from the insertion site are not the same. The mismatch of reading frames can cause frameshift downstream of the insertion site. The other sequence is not particularly limited so long as a sequence that reduces or eliminates the activity of the encoded protein is chosen, and examples thereof include, for example, marker genes such as antibiotic resistance genes, and a gene useful for production of L-glutamic acid.

Disruption of a gene may be performed, specifically, such that an amino acid sequence of an encoded protein is deleted (removed). That is, the modifications that decrease the activity of the protein can be attained by, for example, deleting the amino acid sequence of the protein (a part or the whole of the amino acid sequence), and specifically, by modifying the gene to encode a protein whose amino acid sequence (a part or the whole of the amino acid sequence) is deleted. Incidentally, “deletion of an amino acid sequence of a protein” refers to the deletion of a part or the whole of the amino acid sequence of the protein. Furthermore, “deletion of an amino acid sequence of a protein” means that the original amino acid sequence of the protein no longer exists and also includes a state that the original amino acid sequence is changed to a different amino acid sequence. That is, for example, a region that is changed to a different amino acid sequence due to a frameshift may be regarded as a deleted region. As a result of deletion in the amino acid sequence of the protein, the length of the protein is typically shortened; however, it is also possible for the length of the protein to remain unchanged or even to be extended. For example, by deleting a part or the whole of the coding region of a gene, it is possible to delete the region of the amino acid sequence of the resulting protein corresponding to the deleted region. Furthermore, for example, by introducing a stop codon into the coding region of a gene, it is possible to delete, from the amino acid sequence of the resulting protein, the region that is encoded downstream of the insertion site. Furthermore, for example, by causing a frameshift in the coding region of a gene, it is possible to delete the region corresponding to the frameshift site. The explanations regarding the position and length of the region to be deleted in the case of deletion of the amino acid sequence may be analogously applied to the explanations for the position and length of the region to be deleted in the case of deletion of a gene.

The modification of a gene on a chromosome as described above can be attained by, for example, preparing a deficient type gene modified so that it is unable to produce a protein that normally functions, and transforming a host with a recombinant DNA containing the deficient type gene to cause homologous recombination between the deficient type gene and the wild-type gene on a chromosome and thereby substitute the deficient type gene for the wild-type gene on the chromosome. In this procedure, if a marker gene selected according to the characteristics of the host such as auxotrophy is included in the recombinant DNA, the operation becomes easier. Examples of the deficient type gene include a gene in which a part or the whole of the coding region has been deleted, a gene into which a missense mutation has been introduced, a gene into which a nonsense mutation has been introduced, a gene into which a frameshift mutation has been introduced, and a gene into which insertion sequences such as transposons or marker genes have been inserted. The protein encoded by the deficient type gene has a conformation different from that of the wild-type protein, even if it is produced, and thus the function thereof is reduced or eliminated. The structure of recombinant DNA used for homologous recombination is not particularly limited so long as homologous recombination occurs in the desired manner. For example, the host may be transformed with linear DNA containing the deficient type gene, and the linear DNA may have, at each end, sequences upstream and downstream from the wild-type gene on the chromosome, so that homologous recombination occurs at both the upstream and downstream regions of the wild-type gene, thereby replacing the wild-type gene with the deficient type gene. Such gene disruption based on gene substitution utilizing homologous recombination has already been established, and there are methods of using a linear DNA such as a method called “Red-driven integration” (Datsenko, K. A, and Wanner, B. L., Proc. Natl. Acad. Sci. USA, 97:6640-6645 (2000)), and a method utilizing the Red-driven integration in combination with an excision system derived from λ phage (Cho, E. H., Gumport, R. I., Gardner, J. F., J. Bacteriol., 184:5200-5203 (2002)) (refer to WO2005/010175), a method using a plasmid having a temperature sensitive replication origin, a method using a plasmid capable of conjugative transfer, a method utilizing a suicide vector lacking a replication origin that functions in a host (U.S. Pat. No. 6,303,383, Japanese Patent Laid-open (Kokai) No. 05-007491), and so forth. Incidentally, such modification procedures of chromosomes utilizing homologous recombination are not limited to disruption of target genes, but can be used for arbitrary modifications of chromosomes, such as modification of regulatory sequences.

The modifications that decrease the activity of the protein can also be attained by, for example, a mutagenesis treatment. Examples of the mutagenesis treatment include irradiation of X-ray or ultraviolet and treatment with a mutation agent such as N-methyl-N′-nitro-N-nitrosoguanidine (MNNG), ethyl methanesulfonate (EMS), and methyl methanesulfonate (MMS).

When a protein functions as a complex consisting of a plurality of subunits, all or a part of the plurality of subunits may be modified, so long as the activity of the protein is eventually reduced. That is, for example, a part or all of a plurality of genes that encode the respective subunits may be disrupted or the like. Furthermore, when there is a plurality of isozymes of a protein, a part or all of the activities of the plurality of isozymes may be reduced, so long as the activity of the protein is eventually reduced. That is, for example, a part or all of a plurality of genes that encode the respective isozymes may be disrupted or the like.

The procedures for reducing activity of a protein as described above may be used independently or in any appropriate combination.

A reduction in the activity of a protein can be confirmed by measuring the activity of the protein.

A reduction in the activity of a protein can also be confirmed by confirming a reduction in the expression of a gene encoding the protein. A reduction in the expression of a gene can be confirmed by confirming a reduction in the transcription level of the gene or a reduction in the amount of the protein expressed from the gene.

A reduction in the transcription level of a gene can be confirmed by comparing the amount of mRNA transcribed from the gene with that of a non-modified strain. Examples of the method for confirming the amount of mRNA include Northern hybridization, RT-PCR, microarray, RNA-seq, and so forth (Sambrook, J., et al., Molecular Cloning: A Laboratory Manual/Third Edition, Cold Spring Harbor Laboratory Press, Cold Spring Harbor (USA), 2001). The amount of mRNA (e.g. the number of molecules per cell) may be reduced to, for example, 50% or less, 20% or less, 10% or less, 5% or less, or 0% of that of a non-modified strain.

A reduction in the amount of a protein can be confirmed by Western blotting using antibodies (Sambrook, J., et al., Molecular Cloning: A Laboratory Manual/Third Edition, Cold Spring Harbor Laboratory Press, Cold Spring Harbor (USA), 2001). The amount of the protein (for example, the number of molecules per cell) may be reduced to, for example, 50% or less, 20% or less, 10% or less, 5% or less, or 0% of that of a non-modified strain.

Disruption of a gene can be confirmed by determining nucleotide sequence of apart or the whole of the gene, restriction enzyme map, full length, or the like of the gene depending on the means used for the disruption.

The aforementioned methods for reducing the activity of a protein as mentioned above can be applied to reduction in the activities of arbitrary proteins and reduction in the expression of arbitrary genes.

### <2> Method for Producing L-Glutamic Acid

The method described herein is a method for producing L-glutamic acid, the method comprising:
a step of culturing a coryneform bacterium having an L-glutamic acid-producing ability in a culture medium,
wherein the bacterium is Corynebacterium casei, and
wherein oxygen limitation is implemented during said step.

The aforementioned step (that is, the step of culturing a coryneform bacterium having an L-glutamic acid-producing ability in a culture medium) is also referred to as the “culture step”. Specifically, the culture step may be a step of culturing a coryneform bacterium having an L-glutamic acid-producing ability in a culture medium to obtain a culture product containing L-glutamic acid. The culture product is also referred to as “fermentation broth”.

The culture medium to be used is not particularly limited, so long as the L-glutamic acid-producing bacteria can proliferate in it, and L-glutamic acid can be produced. As the culture medium, for example, a usual culture medium used for culture of coryneform bacteria such as Corynebacterium casei can be used. As the culture medium, for example, a culture medium containing carbon source, nitrogen source, phosphate source, and sulfur source, as well as components selected from other various organic components and inorganic components as required can be used. Types and concentrations of the culture medium components can be appropriately determined according to various conditions such as the type of L-glutamic acid-producing bacteria to be used.

The carbon source is not particularly limited, so long as it can be assimilated by the L-glutamic acid-producing bacteria. Specific examples of the carbon source include, for example, saccharides such as glucose, fructose, sucrose, lactose, galactose, xylose, arabinose, maltose, isomaltose, blackstrap molasses, hydrolysates of starches, and hydrolysates of biomass, organic acids such as acetic acid, fumaric acid, citric acid, and succinic acid, alcohols such as glycerol, crude glycerol, and ethanol, and fatty acids. As a carbon source, sugars composed of fructose may also be used. Examples of the sugars composed of fructose include fructose, sucrose, and fructooligosaccharides. The sugars composed of fructose may be used independently or in combination with other carbon sources as a carbon source. As the carbon source, plant-derived materials can be preferably used. Examples of the plant include, for example, corn, rice, wheat, soybean, sugarcane, beet, and cotton. Examples of the plant-derived materials include, for example, organs such as root, stem, trunk, branch, leaf, flower, and seed, plant bodies including them, and decomposition products of these plant organs. The forms of the plant-derived materials at the time of use thereof are not particularly limited, and they can be used in any form such as unprocessed product, juice, ground product, and purified product. For example, cane molasses, beet molasses, high-test molasses, citrus molasses, or invert sugar can be used as the carbon source; or hydrolysates of natural materials such as cellulose, starch, corn, cereal, tapioca, cassava, and the like may also be used. Pentoses such as xylose, hexoses such as glucose, or mixtures of them can be obtained from, for example, plant biomass, and used. Specifically, these saccharides can be obtained by subjecting a plant biomass to such a treatment as steam treatment, hydrolysis with concentrated acid, hydrolysis with diluted acid, hydrolysis with an enzyme such as cellulase, and alkaline treatment. Since hemicellulose is generally more easily hydrolyzed compared with cellulose, hemicellulose in a plant biomass may be hydrolyzed beforehand to liberate pentoses, and then cellulose may be hydrolyzed to generate hexoses. Furthermore, xylose may be supplied by conversion from hexoses by, for example, imparting a pathway for converting hexose such as glucose to xylose to the L-glutamic acid-producing bacteria. As the carbon source, a single kind of carbon source may be used, or two or more kinds of carbon sources may be used in combination. Specifically, for example, glucose may be used independently, or a mixture of two carbon sources such as glucose and fructose, or glucose and sucrose, at any ratio (for example, a weight ratio of 3:7 to 7:3) may be used.

Specific examples of the nitrogen source include, for example, ammonium salts such as ammonium sulfate, ammonium chloride, and ammonium phosphate, organic nitrogen sources such as peptone, yeast extract, meat extract, and hydrolyzed vegetable protein (HVP; for example, soybean protein decomposition products, soy sauce, pea soy sauce), ammonia, and urea. Ammonia gas or aqueous ammonia used for adjusting pH may also be used as the nitrogen source. Furthermore, in accordance with “Fundamentals of Fermentation Engineering, Academic Society Publishing Center, 1988”, the medium may be reused. As the nitrogen source, a single kind of nitrogen source may be used, or two or more kinds of nitrogen sources may be used in combination.

Specific examples of the phosphate source include, for example, phosphate salts such as potassium dihydrogenphosphate and dipotassium hydrogenphosphate, and phosphate polymers such as pyrophosphate. As the phosphate source, a single kind of phosphate source may be used, or two or more kinds of phosphate sources may be used in combination.

Specific examples of the sulfur source include, for example, inorganic sulfur compounds such as sulfates, thiosulfates, and sulfites, and sulfur-containing amino acids such as cysteine, cystine, and glutathione. As the sulfur source, a single kind of sulfur source may be used, or two or more kinds of sulfur sources may be used in combination.

Specific examples of other various organic components and inorganic components include, for example, inorganic salts such as sodium chloride and potassium chloride; trace metals such as iron, manganese, magnesium, and calcium; vitamins such as vitamin B1, vitamin B2, vitamin B6, nicotinic acid, nicotinamide, vitamin B12, biotin, folic acid; amino acids; nucleic acids; and organic components containing those such as peptone, casamino acids, yeast extract, and hydrolyzed vegetable protein (HVP; for example, soybean protein decomposition products, soy sauce, pea soy sauce). Furthermore, examples of other various organic and inorganic materials may include antifoaming agents, osmotic pressure adjusting agents for culture media, and osmotic pressure compensating agents. Examples of the antifoaming agents include silicone-based antifoaming agents (such as oil-type, solution-type, oil compound-type, emulsion-type, self-emulsifying type), alcohol-based antifoaming agents, oil-based antifoaming agents, polyether-based antifoaming agents, and vegetable oil (such as cottonseed oil, linseed oil, soybean oil, olive oil, castor oil, coconut oil). Examples of the antifoaming agents include any form of antifoaming agents, such as liquid, paste, solid, powder, emulsion, and wax. Examples of the osmotic pressure adjusting agents for culture media include salts such as sodium chloride and potassium chloride, and polysaccharides that are unassimilable by microorganisms (such as sorbitol and dextrin). Examples of the osmotic pressure compensating agents may include potassium ions, betaine (glycine betaine), blackstrap molasses (especially beet blackstrap molasses), glutamic acid, and trehalose. Furthermore, examples of components that may be added to the culture medium may include polymers selected from the group consisting of water-soluble cellulose derivatives, water-soluble polyvinyl compounds, polar organic solvent-soluble polyvinyl compounds, water-soluble starch derivatives, alginate salts, and polyacrylates. As the aforementioned other various organic components and inorganic components, a single kind of component may be used, or two or more kinds of components may be used in combination.

Furthermore, when an auxotrophic mutant strain that requires an amino acid or the like for growth thereof is used, it is preferable to supply a required nutrient to the culture medium.

Furthermore, it is also preferable to, for example, restrict the amount of biotin in the medium, or add a surfactant or penicillin to the culture medium.

The culture conditions are not particularly limited so long as the L-glutamic acid-producing bacterium can proliferate and L-glutamic acid can be produced, except for implementing oxygen limitation during the culture step. The culture can be performed, for example, under usual conditions used for culturing coryneform bacteria such as Corynebacterium casei, except for implementing oxygen limitation during the culture step. The culture conditions can be appropriately set according to various conditions such as the type of the L-glutamic acid-producing bacterium to be used.

The culture can be performed by using a liquid medium (i.e. by liquid culture). Examples of the method of the liquid culture include, for example, the method described in “Biotechnology Textbook Series 13: Culture Engineering, Toshiomi Yoshida, Corona Publishing Co., Ltd., 1998” can be utilized. In other words, examples of liquid culture that may be utilized may include, for example, surface culture, deep culture, membrane (such as dialysis membrane or hollow fiber) separation-type culture, or immobilized microorganism culture. Furthermore, examples of culture apparatuses that may be utilized may include, for example, aeration-agitation type culture devices, air-lift type culture devices, packed-bed culture devices, and fluidized-bed culture devices. For culture, the methods described in “Fundamentals of Fermentation Engineering, Academic Society Publishing Center, 1988” can be used. The culture may be performed separately as seed culture and main culture. L-glutamic acid may be produced in the main culture. Oxygen limitation may be implemented in the main culture. In other words, when culture is performed separately as seed culture and main culture, unless otherwise stated, the term “culture step” refers to the step of culturing the L-glutamic acid-producing bacterium in a culture medium in the main culture. The culture conditions of the seed culture and the main culture may be or may not be the same. At least, the main culture may be performed using a liquid medium. For example, the L-glutamic acid-producing bacterium cultured on a solid medium such as an agar medium may be directly inoculated into a liquid medium, or the L-glutamic acid-producing bacterium subjected to seed culture in a liquid medium may be inoculated into a liquid medium for the main culture. The amount of the L-glutamic acid-producing bacterium contained in the culture medium at the start of culture is not particularly limited. The main culture may be performed by, for example, inoculating a seed culture broth to a culture medium for main culture at an amount of 1 to 50% (v/v). Furthermore, the seed culture step may include, for example, two or more steps of seed culture to obtain the amount of cells required for the main culture. Furthermore, the seed culture may be inoculated only at the start of the main culture, or, in addition to at the start, it may be further inoculated during the main culture.

The culture can be performed as batch culture, fed-batch culture, continuous culture, or a combination thereof. Examples of such combinations may include, for example, cultures in which two or more steps of fed-batch culture are connected, or cultures in which two or more steps of continuous culture are connected. The culture medium used at the time of the start of the culture is also referred to as “starting medium”. The culture medium supplied to a culture system (fermentation tank) in fed-batch culture or continuous culture is also referred to as “feed medium”. Furthermore, to supply a feed medium to a culture system in fed-batch culture or continuous culture is also referred to as to “feed”. Furthermore, when the culture is performed separately as seed culture and main culture, for example, both the seed culture and the main culture may be performed as batch culture. Alternatively, for example, the seed culture may be performed as batch culture, and the main culture may be performed as fed-batch culture or continuous culture. Furthermore, for example, the seed culture may be performed as fed-batch culture, and the main culture may be performed as batch culture. The feed medium may be supplied from a location not in contact with the surface of the liquid medium at the upper part of the culture tank, from an internal position such as the middle or lower part of the culture tank, or from both the upper and middle parts of the culture tank, as appropriate. An embodiment in which the feed medium is supplied from an internal position in the culture medium is disclosed, for example, in Japanese Patent No. 6097869.

The culture medium components each may be contained in the starting medium, the feed medium, or both. The types of the components contained in the starting medium may be or may not be the same as the types of the components contained in the feed medium. The concentration of each component contained in the starting medium may be or may not be the same as the concentration of the corresponding component contained in the feed medium. Furthermore, two or more kinds of feed media containing different types and/or different concentrations of components may be used. For example, when culture medium is intermittently fed a plurality of times, the types and/or concentrations of components contained in the feed media may be or may not be the same for each feeding. For example, the carbon source of the starting medium may be glucose, and the carbon source of the feed medium may be sucrose.

The culture medium may be or may not be sanitized. Sanitization of the culture medium may be performed, for example, for the purpose of preventing contamination by extraneous microorganisms. The term “sanitization” of the culture medium may be interchangeably referred to as “sterilization” or “disinfection”. Examples of the method for sanitizing the culture medium may include sterilization under high-temperature and high-pressure conditions, sterilization by UV irradiation, and disinfection using filters or membranes. Sanitization of the culture medium may be performed in a batchwise manner or in a continuous manner. Examples of the sterilization under high-temperature and high-pressure conditions performed in a batchwise manner include, for example, autoclave sterilization and batch sterilization performed within a culture tank. Furthermore, examples of the sterilization under high-temperature and high-pressure conditions performed in a continuous manner may include continuous sterilization using a plate-type heat exchanger. Furthermore, sanitization of sugars may be performed concurrently with other components of the culture medium or separately from the other components. Preferably, the sugars and the other components may be sanitized separately.

The concentration of the carbon source in the culture medium is not particularly limited, so long as the L-glutamic acid-producing bacterium can proliferate and produce L-glutamic acid. The concentration of the carbon source in the culture medium may be as high as possible within such a range that production of L-glutamic acid is not inhibited. The concentration of the carbon source in the medium may be, as the initial concentration (the concentration in the starting medium), for example, 1 to 50% (w/v), preferably 1 to 30% (w/v), more preferably 3 to 10% (w/v). Furthermore, the carbon source may be additionally supplied to the medium as required. For example, the carbon source may be additionally supplied to the medium in proportion to consumption of the carbon source accompanying progress of the fermentation. Furthermore, in fed-batch culture or continuous culture, the supply amount of the carbon source may be an amount under the condition of sufficiency (i.e. a condition in which a quantity in excess of the carbon assimilation capacity of the L-glutamic acid-producing bacterium is supplied), or may be an amount under the condition of limitation (i.e. a condition in which a quantity less than the carbon assimilation capacity of the L-glutamic acid-producing bacterium is supplied).

The pH of the culture medium may be, for example, pH 3 to 10, preferably pH 4.0 to 9.5. During the culture, the pH of the culture medium may be adjusted as necessary. The pH of the culture medium may be adjusted using various alkaline or acidic substances such as ammonia gas, aqueous ammonia, sodium carbonate, sodium bicarbonate, potassium carbonate, potassium bicarbonate, magnesium carbonate, sodium hydroxide, potassium hydroxide, calcium hydroxide, and magnesium hydroxide. The culture temperature may be, for example, from 20 to 40° C., preferably from 25 to 37° C. The culture period may be, for example, from 10 to 120 hours. The culture may be continued, for example, until the carbon source in the culture medium is consumed or until the activity of the L-glutamic acid-producing bacterium is lost.

When producing L-glutamic acid, culture can also be performed using a liquid medium adjusted to the conditions under which L-glutamic acid precipitates, thereby precipitating L-glutamic acid in the culture medium. The conditions under which glutamic acid precipitates are, for example, pH 5.0 to 4.0, preferably pH 4.5 to 4.0, more preferably pH 4.3 to 4.0, especially preferably pH 4.0 (EP1078989A). When the liquid medium adjusted to the conditions under which L-glutamic acid precipitates is used, it can be crystallised more efficiently by adding pantothenic acid to the culture medium (WO2004/111258). When using the liquid medium adjusted to the conditions under which L-glutamic acid precipitates, crystals of L-glutamic acid can be added to the culture medium as seed crystals for more efficient crystallisation (EP1233069A). When the liquid medium adjusted to the conditions under which L-glutamic acid precipitates is used, crystals of L-glutamic acid and crystals of L-lysine can be added to the medium as seed crystals for more efficient crystallisation (EP1624069A).

In the method described in the present specification, oxygen limitation is implemented during the culture step. In other words, the method described in the present specification may include a step of implementing oxygen limitation during the culture step. This step (i.e. the step of implementing oxygen limitation during the culture step) may also be referred to as the “oxygen limitation step”.

By implementing oxygen limitation, production of L-glutamic acid can be improved as compared to a case where oxygen limitation is not implemented. Therefore, the method described in the present specification may also be interpreted as a method for improving the production of L-glutamic acid.

The embodiments of oxygen limitation (for example, the degree of oxygen limitation, the timing of implementing oxygen limitation, and the length of implementing oxygen limitation) are not particularly limited so long as the production of L-glutamic acid is improved. The embodiments of oxygen limitation can be appropriately set according to various conditions such as the length of the culture period.

The period during which oxygen limitation is implemented (i.e. the culture period under oxygen-limited conditions) may also be referred to as the “oxygen limitation period”. The period during which oxygen limitation is not implemented (i.e. the culture period under non-oxygen-limited conditions) may also be referred to as the “non-oxygen limitation period”. The method described in the present specification (specifically, the culture step) may specifically include the oxygen limitation period. More specifically, the method described in the present specification (specifically, the culture step) may include a non-oxygen limitation period followed by an oxygen limitation period.

“Oxygen limitation” may mean restricting the supply of oxygen to the culture medium. Specifically, “oxygen limitation” may mean restricting the amount of oxygen supplied to the culture medium to a level that is deficient relative to the oxygen consumption capacity of the L-glutamic acid-producing bacterium.

Whether oxygen limitation is implemented or not can be identified by, for example, using the dissolved oxygen concentration in the culture medium as an indicator.

By implementing oxygen limitation, specifically, for example, the culture conditions may become microaerobic conditions. That is, “oxygen limitation” may also mean setting the culture conditions to microaerobic conditions. In other words, culturing during the oxygen limitation period may be implemented under microaerobic conditions. That is, the method described in the present specification (specifically, the culture step) may include a culture period under microaerobic conditions. Furthermore, the “oxygen limitation period” may also mean a culture period under microaerobic conditions.

More specifically, by implementing oxygen limitation, for example, the culture conditions can be switched from aerobic conditions to microaerobic conditions. In other words, “oxygen limitation” may also mean to switch the culture conditions from aerobic conditions to microaerobic conditions. That is, culturing during the non-oxygen limitation period (for example, the period before implementing oxygen limitation) may be performed under aerobic conditions. Furthermore, culturing during the oxygen limitation period may be performed under microaerobic conditions. In other words, the method described in the present specification (specifically, the culture step) may include a culture period under aerobic conditions and a subsequent culture period under microaerobic conditions. Furthermore, “oxygen limitation period” may also mean a culture period under microaerobic conditions. Furthermore, “non-oxygen limitation period” may mean a culture period under aerobic conditions.

The dissolved oxygen concentration in the culture medium during the oxygen limitation period (for example, the culture period under microaerobic conditions) may, for example, fall within the range of dissolved oxygen concentration in the culture medium under microaerobic conditions described below. The dissolved oxygen concentration in the culture medium during the non-oxygen limitation period (for example, the culture period under aerobic conditions) may, for example, fall within the range of dissolved oxygen concentration in the culture medium under aerobic conditions described below. The dissolved oxygen concentration may be measured using a sensor such as a PL electrode or DO electrode.

“Microaerobic conditions” may mean conditions where the dissolved oxygen concentration in the culture medium is less than 0.18 ppm. The dissolved oxygen concentration in the culture medium under microaerobic conditions may be, for example, 0 ppm or more, 0.02 ppm or more, 0.04 ppm or more, 0.06 ppm or more, 0.08 ppm or more, 0.1 ppm or more, 0.12 ppm or more, 0.14 ppm or more, or 0.16 ppm or more, or less than 0.18 ppm, 0.16 ppm or less, 0.14 ppm or less, 0.12 ppm or less, 0.1 ppm or less, 0.08 ppm or less, 0.06 ppm or less, 0.04 ppm or less, or 0.02 ppm or less, or any combination that does not contradict with the above-described ranges. Preferably, the dissolved oxygen concentration in the culture medium under microaerobic conditions may be 0.16 ppm or less, 0.14 ppm or less, 0.12 ppm or less, 0.1 ppm or less, or 0.08 ppm or less. More preferably, the dissolved oxygen concentration in the culture medium under microaerobic conditions may be 0.14 ppm or less, 0.12 ppm or less, 0.1 ppm or less, or 0.08 ppm or less. Particularly preferably, the dissolved oxygen concentration in the culture medium under microaerobic conditions may be 0.12 ppm or less, 0.1 ppm or less, or 0.08 ppm or less. Specifically, the dissolved oxygen concentration in the culture medium under microaerobic conditions may be, for example, from 0 to 0.02 ppm, from 0.02 to 0.04 ppm, from 0.04 to 0.06 ppm, from 0.06 to 0.08 ppm, from 0.08 to 0.1 ppm, from 0.1 to 0.12 ppm, from 0.12 to 0.14 ppm, from 0.14 to 0.16 ppm, or 0.16 ppm or more and less than 0.18 ppm. Specifically, it may be, for example, from 0 to 0.16 ppm, from 0 to 0.14 ppm, from 0 to 0.12 ppm, from 0 to 0.1 ppm, or from 0 to 0.08 ppm. In particular, it may be from 0 to 0.14 ppm, from 0 to 0.12 ppm, from 0 to 0.1 ppm, or from 0 to 0.08 ppm. More specifically, it may be from 0 to 0.12 ppm, from 0 to 0.1 ppm, or from 0 to 0.08 ppm.

“Microaerobic conditions” may also mean conditions under which gene expression from a microaerobically inducible promoter is induced. “Inducing gene expression from a microaerobically inducible promoter” may mean that the expression level of a gene under the control of a microaerobically inducible promoter increases relative to the expression level of the same gene under aerobic conditions. Specifically, “Inducing gene expression from a microaerobically inducible promoter” may mean that, for example, the expression level of a gene under the control of a microaerobically inducible promoter is 1.5 times or more, 2 times or more, or 3 times or more than the expression level of the same gene under aerobic conditions. Examples of the microaerobically inducible promoter may include promoters of genes encoding D- or L-lactate dehydrogenase (e.g., lld, ldhA), promoters of genes encoding alcohol dehydrogenase (e.g., adhE gene), promoters of genes encoding pyruvate formate lyase (e.g., pflB gene), and promoters of genes encoding α-acetolactate decarboxylase (e.g., budA gene).

“Microaerobic conditions” may also mean conditions under which succinate, succinyl-CoA, and/or α-ketoglutaric acid accumulate in the cells and/or in the culture medium. Moreover, “microaerobic conditions” may mean conditions under which the efficiency of cell growth is decreased. “Efficiency of cell growth is decreased” may mean that the efficiency of cell growth is decreased relative to the efficiency of cell growth under aerobic conditions. That is, under microaerobic conditions, supply of oxygen is limited, resulting in inhibition of the metabolic process catalyzed by succinate dehydrogenase (complex II of the respiratory chain-electron transport system), so that metabolites such as succinate (the substrate), its precursor succinyl-CoA, and α-ketoglutaric acid may accumulate. Furthermore, under microaerobic conditions, since progression of the respiratory chain and the associated TCA cycle is inhibited, the efficiency of bioenergetic acquisition may decrease, resulting in reduced cell growth efficiency.

“Aerobic conditions” may mean conditions under which the dissolved oxygen concentration in the culture medium is higher than that under microaerobic conditions. Specifically, “aerobic conditions” may mean conditions under which the dissolved oxygen concentration in the culture medium is higher than the upper limit of the dissolved oxygen concentration range selected as the microaerobic conditions. “Aerobic conditions” may also mean conditions under which the dissolved oxygen concentration in the culture medium is 0.18 ppm or more. Specifically, the dissolved oxygen concentration in the culture medium under aerobic conditions may be, for example, 0.18 ppm or more, 0.2 ppm or more, 0.25 ppm or more, 0.3 ppm or more, 0.5 ppm or more, 1 ppm or more, 1.5 ppm or more, 2 ppm or more, 2.5 ppm or more, 3 ppm or more, 3.5 ppm or more, 4 ppm or more, 4.5 ppm or more, 5 ppm or more, 5.5 ppm or more, 6 ppm or more, 6.5 ppm or more, or 7 ppm or more, or 7.5 ppm or less, 7 ppm or less, 6.5 ppm or less, 6 ppm or less, 5.5 ppm or less, 5 ppm or less, 4.5 ppm or less, 4 ppm or less, 3.5 ppm or less, 3 ppm or less, 2.5 ppm or less, 2 ppm or less, 1.5 ppm or less, 1 ppm or less, 0.5 ppm or less, 0.3 ppm or less, 0.25 ppm or less, or 0.2 ppm or less, or any combination that does not contradict with the above-described ranges. Specifically, the dissolved oxygen concentration in the culture medium under aerobic conditions may be, for example, from 0.18 to 0.2 ppm, from 0.2 to 0.25 ppm, from 0.25 to 0.3 ppm, from 0.3 to 0.5 ppm, from 0.5 to 1 ppm, from 1 to 1.5 ppm, from 1.5 to 2 ppm, from 2 to 2.5 ppm, from 2.5 to 3 ppm, from 3 to 3.5 ppm, from 3.5 to 4 ppm, from 4 to 4.5 ppm, from 4.5 to 5 ppm, from 5 to 5.5 ppm, from 5.5 to 6 ppm, from 6 to 6.5 ppm, from 6.5 to 7 ppm, or from 7 to 7.5 ppm. Specifically, the dissolved oxygen concentration in the culture medium under aerobic conditions may be, for example, from 0.18 to 3.5 ppm, from 0.5 to 3 ppm, or from 1 to 2.5 ppm. However, the dissolved oxygen concentration in the culture medium under aerobic conditions is at most the saturated dissolved oxygen concentration. For example, under atmospheric pressure of 760 mmHg, temperature of 33° C., and water vapor saturated air containing 20.9% oxygen, the saturated dissolved oxygen concentration may be 7.22 ppm.

Whether or not oxygen limitation is implemented can also be identified by using Rab (Ratio of absorption) as an indicator. “Rab (Ratio of absorption)” refers to the oxygen consumption rate.

“Oxygen limitation” may also mean limiting Rab to a predetermined range (for example, the range described below).

During the oxygen limitation period (for example, the culture period under microaerobic conditions), Rab per OD620 may be, for example, 0.06 mol(O₂)/min/mL or more, 0.065 mol(O₂)/min/mL or more, 0.07 mol(O₂)/min/mL or more, 0.075 mol(O₂)/min/mL or more, 0.08 mol(O₂)/min/mL or more, 0.1 mol(O₂)/min/mL or more, 0.12 mol(O₂)/min/mL or more, 0.14 mol(O₂)/min/mL or more, 0.16 mol(O₂)/min/mL or more, 0.18 mol(O₂)/min/mL or more, 0.2 mol(O₂)/min/mL or more, 0.22 mol(O₂)/min/mL or more, 0.24 mol(O₂)/min/mL or more, 0.26 mol(O₂)/min/mL or more, 0.28 mol(O₂)/min/mL or more, 0.3 mol(O₂)/min/mL or more, 0.32 mol(O₂)/min/mL or more, 0.34 mol(O₂)/min/mL or more, 0.36 mol(O₂)/min/mL or more, or 0.38 mol(O₂)/min/mL or more, or 0.4 mol(O₂)/min/mL or less, 0.38 mol(O₂)/min/mL or less, 0.36 mol(O₂)/min/mL or less, 0.34 mol(O₂)/min/mL or less, 0.32 mol(O₂)/min/mL or less, 0.3 mol(O₂)/min/mL or less, 0.28 mol(O₂)/min/mL or less, 0.26 mol(O₂)/min/mL or less, 0.24 mol(O₂)/min/mL or less, 0.22 mol(O₂)/min/mL or less, 0.2 mol(O₂)/min/mL or less, 0.18 mol(O₂)/min/mL or less, 0.16 mol(O₂)/min/mL or less, 0.14 mol(O₂)/min/mL or less, 0.12 mol(O₂)/min/mL or less, 0.1 mol(O₂)/min/mL or less, 0.08 mol(O₂)/min/mL or less, 0.075 mol(O₂)/min/mL or less, 0.07 mol(O₂)/min/mL or less, or 0.065 mol(O₂)/min/mL or less, or any combination that does not contradict with the above-described ranges. Specifically, Rab per OD620 during the oxygen limitation period (for example, the culture period under microaerobic conditions) may be, for example, from 0.06 to 0.065 mol(O₂)/min/mL, from 0.065 to 0.07 mol(O₂)/min/mL, from 0.07 to 0.075 mol(O₂)/min/mL, from 0.075 to 0.08 mol(O₂)/min/mL, from 0.08 to 0.1 mol(O₂)/min/mL, from 0.1 to 0.12 mol(O₂)/min/mL, from 0.12 to 0.14 mol(O₂)/min/mL, from 0.14 to 0.16 mol(O₂)/min/mL, from 0.16 to 0.18 mol(O₂)/min/mL, from 0.18 to 0.2 mol(O₂)/min/mL, from 0.2 to 0.22 mol(O₂)/min/mL, from 0.22 to 0.24 mol(O₂)/min/mL, from 0.24 to 0.26 mol(O₂)/min/mL, from 0.26 to 0.28 mol(O₂)/min/mL, from 0.28 to 0.3 mol(O₂)/min/mL, from 0.3 to 0.32 mol(O₂)/min/mL, from 0.32 to 0.34 mol(O₂)/min/mL, from 0.34 to 0.36 mol(02)/min/mL, from 0.36 to 0.38 mol(O₂)/min/mL, or from 0.38 to 0.4 mol(O₂)/min/mL. Specifically, Rab per OD620 during the oxygen limitation period (for example, the culture period under microaerobic conditions) may be, for example, from 0.06 to 0.4 mol(O₂)/min/mL, from 0.075 to 0.4 mol(O₂)/min/mL, from 0.1 to 0.4 mol(O₂)/min/mL, from 0.06 to 0.3 mol(O₂)/min/mL, from 0.08 to 0.3 mol(O₂)/min/mL, from 0.1 to 0.3 mol(O₂)/min/mL, from 0.1 to 0.26 mol(O₂)/min/mL, or from 0.1 to 0.24 mol(O₂)/min/mL. Specifically, Rab per OD620 during the oxygen limitation period (for example, the culture period under microaerobic conditions) may be from 0.1 to 0.3 mol(O₂)/min/mL. For example, Rab may be calculated based on the oxygen concentration in the feed gas and the exhaust gas.

“OD620” refers to the optical density of the culture liquid at a measurement wavelength of 620 nm. For example, OD620 may be measured using a spectrophotometer. OD620 may be calculated from the dry cell weight. Specifically, for C. casei, the following relation is valid: dry cell weight (g/L)=(OD620×0.233)−2.97, and based on this relation OD620 and dry cell weight can be mutually converted. “Dry cell weight (g/L)” means the dry weight (g) of cells contained in 1 L of the culture liquid. The dry cell weight can be measured by separating cells from the culture liquid, drying them, and measuring the weight of the dried product obtained.

During the oxygen limitation period (for example, the culture period under microaerobic conditions), Rab may be, for example, 1 mol(O₂)/min/mL or more, 2 mol(O₂)/min/mL or more, 3 mol(O₂)/min/mL or more, 4 mol(O₂)/min/mL or more, 5 mol(O₂)/min/mL or more, 6 mol(O₂)/min/mL or more, 7 mol(O₂)/min/mL or more, 8 mol(O₂)/min/mL or more, 9 mol(O₂)/min/mL or more, 10 mol(O₂)/min/mL or more, 12 mol(O₂)/min/mL or more, 15 mol(O₂)/min/mL or more, 20 mol(O₂)/min/mL or more, or 25 mol(O₂)/min/mL or more, or 30 mol(O₂)/min/mL or less, 25 mol(O₂)/min/mL or less, 20 mol(O₂)/min/mL or less, 15 mol(O₂)/min/mL or less, 12 mol(O₂)/min/mL or less, 10 mol(O₂)/min/mL or less, 9 mol(O₂)/min/mL or less, 8 mol(O₂)/min/mL or less, 7 mol(O₂)/min/mL or less, 6 mol(O₂)/min/mL or less, 5 mol(O₂)/min/mL or less, 4 mol(O₂)/min/mL or less, 3 mol(O₂)/min/mL or less, or 2 mol(O₂)/min/mL or less, or any combination that does not contradict with the above-described ranges. Specifically, Rab during the oxygen limitation period (for example, the culture period under microaerobic conditions) may be, for example, from 1 to 2 mol(O₂)/min/mL, from 2 to 3 mol(O₂)/min/mL, from 3 to 4 mol(O₂)/min/mL, from 4 to 5 mol(O₂)/min/mL, from 5 to 6 mol(O₂)/min/mL, from 6 to 7 mol(O₂)/min/mL, from 7 to 8 mol(O₂)/min/mL, from 8 to 9 mol(O₂)/min/mL, from 9 to 10 mol(O₂)/min/mL, from 10 to 12 mol(O₂)/min/mL, from 12 to 15 mol(O₂)/min/mL, from 15 to 20 mol(O₂)/min/mL, from 20 to 25 mol(O₂)/min/mL, or from 25 to 30 mol(O₂)/min/mL. Specifically, Rab during the oxygen limitation period (for example, the culture period under microaerobic conditions) may be, for example, from 1 to 30 mol(O₂)/min/mL, from 3 to 25 mol(O₂)/min/mL, or from 5 to 20 mol(O₂)/min/mL.

The timing for implementing (i.e. starting) oxygen limitation can be appropriately set according to various conditions such as the length of a culture period. The timing for implementing (starting) oxygen limitation may be, for example, a point after a predetermined period has elapsed from the start of culture. The length of the predetermined period referred to herein may be, for example, 5 hours or more, 7 hours or more, 10 hours or more, 15 hours or more, 20 hours or more, 25 hours or more, or 30 hours or more; or it may also be 5 hours or more, 7 hours or more, 10 hours or more, 15 hours or more, 20 hours or more, 25 hours or more, or 30 hours or more, or 40 hours or less, 30 hours or less, 25 hours or less, 20 hours or less, 15 hours or less, 10 hours or less, or 7 hours or less, or any combination that does not contradict with the above-described ranges. Specifically, the length of the predetermined period may be, for example, from 5 to 7 hours, from 7 to 10 hours, from 10 to 15 hours, from 15 to 20 hours, from 20 to 25 hours, from 25 to 30 hours, or from 30 to 40 hours. Specifically, the length of the predetermined period may be, for example, from 5 to 40 hours, from 5 to 30 hours, or from 5 to 20 hours. Furthermore, the timing for performing (starting) oxygen limitation may be a point when OD620 is within a predetermined range. The predetermined OD620 range referred to herein may be, for example, 10 or more, 15 or more, 20 or more, 25 or more, 30 or more, 35 or more, 40 or more, 50 or more, or 70 or more, or 100 or less, 70 or less, 50 or less, 40 or less, 35 or less, 30 or less, 25 or less, 20 or less, or 15 or less, or any combination that does not contradict with the above-described ranges. Specifically, the predetermined OD620 range may be, for example, from 10 to 15, from 15 to 20, from 20 to 25, from 25 to 30, from 30 to 35, from 35 to 40, from 40 to 50, from 50 to 70, or from 70 to 100. Specifically, the predetermined OD620 range may be, for example, from 20 to 100, from 25 to 70, or from 30 to 50. Incidentally, “implementing oxygen limitation at a certain timing” means implementing (i.e. starting) oxygen limitation at least at that timing and does not preclude implementing oxygen limitation (i.e. starting) at times other than that timing.

After the start of oxygen limitation, an oxygen-limited state may be maintained throughout the entire culture period, or it may be maintained only during part of the culture period. That is, for example, after the start of oxygen limitation, the oxygen limitation may be ended before completion of culture. Alternatively, after the start of oxygen limitation, the oxygen limitation may be once stopped and then restarted. Furthermore, after the start of oxygen limitation, stopping and restarting of the oxygen limitation may be repeated. In other words, oxygen limitation may be implemented intermittently multiple times. The period of the culture during which the oxygen-limited state is maintained may be set so that, for example, the length of the oxygen limitation period described below is obtained. Preferably, after the start of oxygen limitation, the oxygen-limited state is maintained during the entire culture period (i.e. until the end of culture). “Maintaining oxygen-limited state” may mean, for example, maintaining microaerobic conditions. Furthermore, “maintaining oxygen-limited state” may mean maintaining Rab in the ranges exemplified above. Furthermore, “maintaining oxygen-limited state” may mean maintaining microaerobic conditions and, at the same time, maintaining Rab in the ranges exemplified above. By stopping or ending oxygen limitation, for example, the culture conditions may become aerobic conditions. By stopping or ending oxygen limitation, the culture conditions may be switched from microaerobic conditions to aerobic conditions.

The length of the oxygen limitation period can be appropriately set according to various conditions such as the timing of oxygen limitation and the length of the culture period. The length of the oxygen limitation period may be, for example, 70% or more, 80% or more, 90% or more, 95% or more, 97% or more, or 99% or more of the entire period after the start of the oxygen limitation until the end of culture. Furthermore, the oxygen limitation period may be, for example, 70% or more, 80% or more, 90% or more, 95% or more, 97% or more, or 99% or more of the period from the start of the oxygen limitation until complete consumption of the carbon source. Furthermore, the oxygen limitation period may be, for example, 70% or more, 80% or more, 90% or more, 95% or more, 97% or more, or 99% or more of the period during which OD620 is 10 or more, 15 or more, 20 or more, 25 or more, 30 or more, 35 or more, or 40 or more. Furthermore, the oxygen limitation period may be, for example, 1 hour or more, 2 hours or more, 3 hours or more, 5 hours or more, 7 hours or more, 10 hours or more, 15 hours or more, 20 hours or more, 25 hours or more, or 30 hours or more. “Length of the oxygen limitation period” refers to the total length during which oxygen limitation is implemented, in a case where the oxygen limitation is implemented intermittently multiple times. Preferably, after the start of the oxygen limitation, the oxygen-limited state is maintained throughout a period corresponding to the length of the oxygen limitation period exemplified above, without being stopped.

During the oxygen limitation period, oxygen is supplied to the culture medium. That is, for example, oxygen may be supplied to the culture medium so that the microaerobic conditions exemplified above are maintained during the oxygen limitation period. Furthermore, for example, oxygen may be supplied so that Rab is maintained within the ranges exemplified above during the oxygen limitation period. Furthermore, oxygen may be supplied to the culture medium so as to maintain both the microaerobic conditions and Rab within the ranges exemplified above. Oxygen supply may be implemented during the entire oxygen limitation period or only during part of the period. That is, for example, oxygen supply may be terminated prior to completion of the culture during the oxygen limitation period. Alternatively, during the oxygen limitation period, oxygen supply may be temporarily stopped and then restarted. Furthermore, oxygen supply may be stopped and restarted multiple times during the oxygen limitation period. In other words, oxygen supply may be performed intermittently multiple times. Preferably, oxygen supply is continued throughout the entire oxygen limitation period. The length of oxygen supply may be 70% or more, 80% or more, 90% or more, 95% or more, 97% or more, or 99% or more of the oxygen limitation period.

The supply of oxygen (for example, oxygen supply during the non-oxygen limitation period and the oxygen limitation period) can be implemented, for example, by aeration of the culture medium, shaking of the culture medium, agitation of the culture medium, or a combination thereof. In other words, culture may be implemented, for example, by aerated culture, shaken culture, agitated culture, or a combination thereof. The oxygen limitation may be implemented, for example, by reducing the rate or amount of aeration of the culture medium, reducing the shaking speed of the culture medium, reducing the agitating speed of the culture medium, or a combination thereof. Oxygen may be supplied to the culture medium alone or as part of mixed gas with other gases. The ratio of oxygen in the mixed gas may be, for example, from 10 to 30 v/v %, preferably about 20 v/v %. Specific examples of mixed gas include air.

During the culture step, the temperature of the culture medium may be increased. In other words, the method described in the present specification may further include a step of increasing the temperature of the culture medium during the culture step. This step (i.e. the step of increasing the temperature of the culture medium during the culture step) may also be referred to as the “temperature-increase step”. Increasing the temperature of the culture medium may also be referred to as “temperature elevation” or “elevating the temperature of the culture medium”.

The embodiment of elevating the temperature of the culture medium is not particularly limited so long as L-glutamic acid can be produced. The embodiment of elevating the temperature of the culture medium may be, for example, set so that the production of L-glutamic acid is improved as compared with a case where the temperature elevation is not implemented. The embodiment of the temperature elevation may be appropriately set according to various conditions such as the length of the culture period.

The amount of the temperature elevation of the culture medium (i.e. the difference between the temperature before and after elevation) may be, for example, 1° C. or more, 2° C. or more, 3° C. or more, 4° C. or more, 5° C. or more, 6° C. or more, 7° C. or more, 8° C. or more, 9° C. or more, 10° C. or more, or 12° C. or more, or 15° C. or less, 12° C. or less, 10° C. or less, 9° C. or less, 8° C. or less, 7° C. or less, 6° C. or less, 5° C. or less, 4° C. or less, 3° C. or less, or 2° C. or less, or any combination that does not contradict with the above-described ranges. Specifically, the amount of the temperature elevation (i.e. the difference between the temperature before and after elevation) may be, for example, from 1 to 2° C., from 2 to 3° C., from 3 to 4° C., from 4 to 5° C., from 5 to 6° C., from 6 to 7° C., from 7 to 8° C., from 8 to 9° C., from 9 to 10° C., from 10 to 12° C., or from 12 to 15° C. Specifically, the amount of the temperature elevation (i.e. the difference between the temperature before and after elevation) may be, for example, from 1 to 12° C., from 2 to 9° C., or from 3 to 6° C.

The temperature of the culture medium before the temperature elevation may be, for example, 20° C. or more, 25° C. or more, 27° C. or more, 28° C. or more, 29° C. or more, 30° C. or more, or 31° C. or more, or 32° C. or less, 31° C. or less, 30° C. or less, 29° C. or less, 28° C. or less, 27° C. or less, or 25° C. or less, or any combination that does not contradict with the above-described ranges. Specifically, the temperature of the culture medium before the temperature elevation may be, for example, from 20 to 25° C., from 25 to 27° C., from 27 to 28° C., from 28 to 29° C., from 29 to 30° C., from 30 to 31° C., or from 31 to 32° C. Specifically, the temperature of the culture medium before the temperature elevation may be, for example, from 20 to 32° C., from 25 to 32° C., or from 27 to 32° C.

The temperature of the culture medium after the temperature elevation may be, for example, 32° C. or more, 33° C. or more, 34° C. or more, 35° C. or more, 36° C. or more, 37° C. or more, 38° C. or more, or 39° C. or more, or 40° C. or less, 39° C. or less, 38° C. or less, 37° C. or less, 36° C. or less, 35° C. or less, 34° C. or less, or 33° C. or less, or any combination that does not contradict with the above-described ranges. Specifically, the temperature of the culture medium after the temperature elevation may be, for example, from 32 to 33° C., from 33 to 34° C., from 34 to 35° C., from 35 to 36° C., from 36 to 37° C., from 37 to 38° C., from 38 to 39° C., or from 39 to 40° C. Specifically, the temperature of the culture medium after the temperature elevation may be, for example, from 32 to 40° C., from 33 to 38° C., or from 34 to 36° C.

The timing for implementing the temperature elevation can be appropriately set according to various conditions such as the length of the culture period. The timing for the temperature elevation may be the same as or different from that of oxygen limitation. The timing for the temperature elevation may be prior to, simultaneous with, or after the timing of oxygen limitation. The timing for the temperature elevation may be within 5 hours, within 2 hours, or within 1 hour before or after the timing of oxygen limitation. The timing for the temperature elevation may be, for example, a point after a predetermined period has elapsed since the start of culture. The length of the predetermined period may be, for example, 5 hours or more, 7 hours or more, 10 hours or more, 15 hours or more, 20 hours or more, 25 hours or more, or 30 hours or more; and it may also be 5 hours or more, 7 hours or more, 10 hours or more, 15 hours or more, 20 hours or more, 25 hours or more, or 30 hours or more, or 40 hours or less, 30 hours or less, 25 hours or less, 20 hours or less, 15 hours or less, 10 hours or less, or 7 hours or less, or any combination that does not contradict with the above-described ranges. Specifically, the predetermined period may be, for example, from 5 to 7 hours, from 7 to 10 hours, from 10 to 15 hours, from 15 to 20 hours, from 20 to 25 hours, from 25 to 30 hours, or from 30 to 40 hours. Specifically, the predetermined period may be from 5 to 40 hours, from 5 to 30 hours, or from 5 to 20 hours. Furthermore, the timing for the temperature elevation may be a point when OD620 is within a predetermined range. The predetermined OD620 range may be, for example, 10 or more, 15 or more, 20 or more, 25 or more, 30 or more, 35 or more, 40 or more, 50 or more, or 70 or more, or 100 or less, 70 or less, 50 or less, 40 or less, 35 or less, 30 or less, 25 or less, 20 or less, or 15 or less, or any combination that does not contradict with the above-described ranges. Specifically, the predetermined OD620 range may be, for example, from 10 to 15, from 15 to 20, from 20 to 25, from 25 to 30, from 30 to 35, from 35 to 40, from 40 to 50, from 50 to 70, or from 70 to 100. Specifically, the predetermined OD620 range may be from 20 to 100, from 25 to 70, or from 30 to 50. “Implementing temperature elevation at a certain timing” means performing temperature elevation at least at that timing and does not preclude implementing additional temperature elevation at other timings.

After the temperature elevation, the elevated temperature may be maintained throughout the entire culture period or only during a part of the culture period. That is, for example, after the temperature elevation, the temperature of the culture medium may become outside of the range of the elevated temperature (for example, to a temperature lower than the elevated temperature) before the end of the culture. Furthermore, after the temperature elevation, the temperature of the culture medium may become outside of the range of the elevated temperature (for example, to a temperature lower than the elevated temperature) and then return to within the range of the elevated temperature. Furthermore, for example, after the temperature elevation, the temperature of the culture medium may go back and forth between being within and outside the range of the elevated temperature (for example, to a temperature lower than the elevated temperature) multiple times. In other words, the temperature elevation may be implemented intermittently multiple times. The period of the culture for which the elevated temperature is maintained may be set so that, for example, the length of the period during which the elevated temperature is maintained as described below is obtained. Preferably, after the temperature elevation, the elevated temperature is maintained throughout the entire culture period (i.e. until the end of culture). “Maintaining the elevated temperature” may mean, for example, maintaining the temperature of the culture medium within the temperature range exemplified above after the elevation.

The length of the period during which the elevated temperature is maintained can be appropriately set according to various conditions such as the timing of temperature elevation and the length of the culture period. The length of the period during which the elevated temperature is maintained may be, for example, 70% or more, 80% or more, 90% or more, 95% or more, 97% or more, or 99% or more of the entire culture period after temperature elevation. Furthermore, the length of the period during which the elevated temperature is maintained may be, for example, 70% or more, 80% or more, 90% or more, 95% or more, 97% or more, or 99% or more of the period from temperature elevation until complete consumption of the carbon source. The length of the period during which the elevated temperature is maintained may also be 70% or more, 80% or more, 90% or more, 95% or more, 97% or more, or 99% or more of the period during which OD620 is 10 or more, 15 or more, 20 or more, 25 or more, 30 or more, 35 or more, or 40 or more. Furthermore, the period may be 1 hour or more, 2 hours or more, 3 hours or more, 5 hours or more, 7 hours or more, 10 hours or more, 15 hours or more, 20 hours or more, 25 hours or more, or 30 hours or more. “The length of the period during which the elevated temperature is maintained” refers to the total length during which the elevated temperature is maintained in a case where temperature elevation is implemented intermittently multiple times. Preferably, after temperature elevation, the elevated temperature is maintained during a period corresponding to the length exemplified above, without allowing the temperature of the culture medium to deviate from the range after temperature elevation.

In this manner, by culturing the L-glutamic acid-producing bacterium, L-glutamic acid accumulates in the culture medium and/or in the cells, and thus a culture product containing L-glutamic acid (i.e. fermentation broth) is obtained.

Production of L-glutamic acid can be confirmed by known procedures used for detection or identification of compounds. Examples of such procedures may include, for example, HPLC, LC/MS, GC/MS, and NMR. These procedures can be independently used, or can be used in an appropriate combination.

The fermentation broth can be treated, for example, using a liquid cyclone. Examples of the liquid cyclone that may be used may include, for example, generally shaped cyclones made of ceramic, stainless steel, or resin with a cylindrical section diameter of 10 to 110 mm. The feed rate of the fermentation broth into the liquid cyclone may be set, for example, according to the cell concentration or the concentration of L-glutamic acid in the fermentation broth. The feed rate of the fermentation broth into the liquid cyclone may be, for example, from 2 to 1200 L/min.

L-glutamic acid may be produced as a composition containing L-glutamic acid (for example, obtained and utilized as such). In other words, the produced L-glutamic acid may be in the form of a composition containing L-glutamic acid. The composition containing L-glutamic acid may consist solely of L-glutamic acid, or may comprise components other than L-glutamic acid. There are no particular restrictions on the components other than L-glutamic acid. The components other than L-glutamic acid may be appropriately selected, for example, according to various conditions such as the intended use of L-glutamic acid. Components other than L-glutamic acid may be derived from, for example, a culture product containing L-glutamic acid (i.e. a fermentation broth), or may be separately incorporated. Examples of the components other than L-glutamic acid may include bacterial cells, culture medium components, moisture, and bacterial metabolic by-products. Further examples of the components other than L-glutamic acid may include ingredients blended into food or pharmaceuticals. Specific examples of the ingredients blended into food or pharmaceuticals may include excipients and other additives.

L-glutamic acid may be produced, for example, as a culture product containing L-glutamic acid (i.e. a fermentation broth) (for example, obtained and used). In other words, the produced L-glutamic acid (for example, a composition containing L-glutamic acid) may be, for example, a culture product containing L-glutamic acid (i.e. a fermentation broth). Furthermore, for example, L-glutamic acid may also be collected from a culture product (i.e. a fermentation broth). In other words, the method described herein may further include a step of collecting L-glutamic acid from a culture product (i.e. a fermentation broth). L-glutamic acid may be collected as an appropriate fraction containing L-glutamic acid. Examples of such fraction may include, for example, the culture supernatant. The culture supernatant may be obtained, for example, by subjecting the culture to centrifugation. Furthermore, L-glutamic acid may also be isolated and purified from such fractions as described above. For example, after separating the cells of the L-glutamic acid-producing bacteria from the culture product to obtain the culture supernatant, L-glutamic acid may be collected from the culture supernatant. Furthermore, in a case where L-glutamic acid accumulates within the cells, for example, the cells may be disrupted by ultrasonic treatment or the like, the supernatant may be obtained by centrifugation or the like, and L-glutamic acid may be collected from the supernatant. L-glutamic acid can be collected by known procedures used for separation and purification of compounds. Examples of such procedures may include, for example, ion-exchange resin method (Nagai, H. et al., Separation Science and Technology, 39(16), 3691-3710), precipitation, membrane separation (Japanese Patent Laid-open (Kokai) No. 9-164323 and Japanese Patent Laid-open (Kokai) No. 9-173792), and crystallization (WO2008/078448 and WO2008/078646). For example, when L-glutamic acid precipitates in the culture medium, L-glutamic acid may be collected from the culture medium by centrifugation or filtration. Furthermore, L-glutamic acid that has precipitated in the culture medium may be collected from the culture medium together with L-glutamic acid dissolved in the culture medium after crystallization. Furthermore, fractions containing L-glutamic acid (for example, the culture product or culture supernatant) may be appropriately processed prior to use. Examples of such processing may include concentration, drying, and heating. Concentration, drying, and heating may each be performed by any known procedures. Examples of the known procedures of drying may include spray drying and freeze drying. In other words, the produced L-glutamic acid (for example, a composition containing L-glutamic acid) may include the culture product of the L-glutamic acid-producing bacterium, the culture supernatant collected therefrom, the processed products thereof, or L-glutamic acid collected therefrom. Specific examples of the processed products may include dried products (for example, a dried product of the culture product or a dried product of the supernatant of the culture product). Specific examples of the dried products of the culture product may include dried powders (for example, dried culture powders or dried culture supernatant powders). The dried products (for example, dried powders) may be or may not be subjected to processing other than drying. The dried products (for example, dried powders) may contain or may not contain components other than L-glutamic acid (for example, excipients or other additives).

The produced L-glutamic acid may be in a free form, a salt thereof, or a mixture thereof. The produced L-glutamic acid may specifically be L-glutamic acid in a free form, sodium L-glutamate (for example, monosodium L-glutamate; MSG), potassium L-glutamate (for example, monopotassium L-glutamate), ammonium L-glutamate (for example, monoammonium L-glutamate), or a mixture thereof. For example, ammonium L-glutamate present in a fermentation broth can be crystallized by adding acid, and monosodium L-glutamate (MSG) can be obtained by adding an equimolar amount of sodium hydroxide to the resulting crystals. Furthermore, decolorization may be performed by adding activated carbon before or after crystallization (see “Industrial Crystallization of Monosodium Glutamate,” Journal of the Japan Society for Salt Science and Technology, Vol. 56, No. 5, Tetsuya Kawakita). The crystals of sodium L-glutamate may be used, for example, as an umami seasoning. The sodium L-glutamate crystals may also be used as a seasoning in combination with nucleic acids such as sodium guanylate or sodium inosinate, which similarly impart umami taste.

The collected L-glutamic acid may contain components such as bacterial cells, medium components, moisture, and by-product metabolites of the bacterium in addition to L-glutamic acid. The collected L-glutamic acid may also be purified to a desired extent. Purity of the collected L-glutamic acid may be, for example, 50% (w/w) or higher, preferably 85% (w/w) or higher, particularly preferably 95% (w/w) or higher (JP1214636B, U.S. Pat. Nos. 5,431,933, 4,956,471, 4,777,051, 4,946,654, 5,840,358, 6,238,714, and US2005/0025878).

The produced L-glutamic acid may be used as is, or in combination with components other than L-glutamic acid. Specifically, the produced L-glutamic acid may be used as is, or in combination with components other than L-glutamic acid, as a composition containing L-glutamic acid. Furthermore, during the production of L-glutamic acid (for example, during the processing of a fraction containing L-glutamic acid), components other than L-glutamic acid may be incorporated. For example, additives such as excipients may be incorporated during processing such as drying.

The use of the produced L-glutamic acid is not particularly limited. The produced L-glutamic acid may be used, for example, as a seasoning. In other words, the produced L-glutamic acid (for example, a composition containing L-glutamic acid) may be a seasoning. The produced L-glutamic acid may be used, for example, to enhance the umami taste of food products. In other words, the produced L-glutamic acid (for example, a composition containing L-glutamic acid) may be a composition for enhancing the umami taste of food products.

### EXAMPLES

Hereafter, the present invention will be more specifically explained with reference to examples. However, the present invention is not limited by these examples. In the present embodiment, “L-Glu” or “Glu” refers to L-glutamic acid. In the present embodiment, “CT” or “C.T.” refers to Culturing Time.

### Example 1: Obtaining High L-Glutamic Acid-Producing Strains from Corynebacterium casei JCM 12072

(1) Production of Mutant Strain Library of C. casei JCM 12072

Cells of C. casei JCM 12072 were inoculated in a Sakaguchi flask where 30 mL of a medium described in Table 2(A) was placed, and shaken and cultured at 30° C. for one day, and then the cells were collected. The cell was suspended in a solution containing 0.1 M potassium phosphate buffer (pH 7.0), 6.0% dimethyl sulfoxide, and 0.1 mg/mL N-methyl-N-nitrosoguanidine (NTG), and left to stand at room temperature for 50 minutes. The cell was collected and washed with 0.1 M potassium phosphate buffer (pH 7.0) three times. It was cultured at 30° C. for 2 hours in a Sakaguchi flask where 30 mL of a medium for recovery culture described in Table 2(B) was placed, and the cell was collected. The cell was suspended in 20% glycerol, and stored at −80° C. This was adopted as a mutant strain library.

TABLE 2
(A)

| Component | Final Concentration |
|---|---|
| Soy peptone | 40 g/L |
| Yeast extract | 8 g/L |
| MgSO₄•7H₂O | 1 g/L |

Autoclave sterilization at pH 7.5 (KOH) and 120° C. for 20 minutes
(B)

| Component | Final Concentration |
|---|---|
| Soy peptone | 40 g/L |
| Yeast extract | 8 g/L |
| MgSO₄•7H₂O | 1 g/L |
| Na₂C₄HAO₄•6H₂O | 2 g/L |

Autoclave sterilization at pH 7.0 (KOH) and 120° C. for 20 minutes

(2) Screening of High L-Glutamic Acid-Producing Strains from Mutant Strain Library

Next, the mutant strain library prepared was shaken and cultured at 30° C. for 29 hours in a Glu-minimal medium described in Table 3 to which 100 mg/L ampicillin was added, and then seeded on an agar medium described in Table 2(A). Colonies grown were inoculated respectively to the Glu-minimal medium described in Table 3 and the agar medium described in Table 2(A), and 26 clone strains lowered in ability to assimilate L-glutamic acid (L-Glu) were selected as candidate strains.

TABLE 3

| Component | Final Concentration | |
|---|---|---|
| KH₂PO₄ | 3 | g/L |
| NaCl | 0.5 | g/L |
| Na₂HPO₄ | 6.78 | g/L |
| NH₄Cl | 1 | g/L |
| Sodium hydrogen L(+)-glutamate monohydrate | 4.49 | g/L |
| MgSO₄ | 0.24 | g/L |
| CaCl₂ | 0.01 | g/L |
| Biotin | 0.1 | μg/L |

pH not adjusted, filter sterilization

Twenty six strains as the candidate strains were shaken and cultured at 30° C. for 48 hours in a 500 μL evaluation medium (Table 4) contained in a 96 deep well plate, and the amount of L-Glu accumulated in the medium was measured with Biotec Analyzer AS210 (SAKURA SI CO., LTD.). RUN5-2-96 strain (NITE BP-03688) having the highest L-Glu concentration among the strains evaluated was selected. RUN5-2-96 strain is also referred to as AJ111891 strain. RUN5-2-96 strain has all of 135 mutations of Group A (i.e. all of mutations A-1 to A-135).

TABLE 4
< A zone >

| Component | Final Concentration | |
|---|---|---|
| Glucose | 55 | g/L |

Autoclave sterilization at pH not adjusted and 120° C. for 20 minutes
< B zone >

| Component | Final Concentration | |
|---|---|---|
| (NH₄)₂SO₄ | 5 | g/L |
| KH₂PO₄ | 1 | g/L |
| MgSO₄•7H₂O | 0.4 | g/L |
| FeSO₄•7H₂O | 0.01 | g/L |
| MnSO₄•5H₂O | 0.01 | g/L |
| VB1 | 0.0002 | g/L |
| Biotin | 0.0006 | g/L |
| Bacto Peptone | 10 | g/L |
| Yeast Extract | 5 | g/L |

Autoclave sterilization at pH 7.5 (KOH) and 120° C. for 20 minutes

### Mixed at Ratio of 20% of a Zone and 80% of B Zone

(3) Screening of High L-Glutamic Acid-Producing Strains from the Mutant Strain Library

Separately, A-013 strain (NITE BP-03806) was selected from the mutant strain library prepared in (1) as a high Glu-producing strain; A-013 strain is also referred to as AJ120306 strain.

### Example 2: Evaluation of the Impact of Increased Culture Temperature and/or Oxygen Limitation on Glu Production During Culture of C. casei JCM 12072 (Wild-Type Strain)

In this example, the impact of increased culture temperature and/or oxygen limitation on Glu production was evaluated during culture of C. casei JCM 12072 (wild-type strain; WT). The increase in culture temperature is also referred to simply as “temperature elevation”.

First, a seed culture of C. casei JCM 12072 (wild-type strain) was performed via jar fermentation using the seed culture medium shown in Table 6. The culture temperature was maintained constantly at 30° C., and the culture pH was controlled at 6.8 with ammonia gas.

Next, main culture of C. casei JCM 12072 (wild-type strain) was performed via jar fermentation using the culture medium described in Table 7. The culture pH was maintained at 6.8 using ammonia gas, as with the seed culture. After inoculation with 1.5% seed culture broth to start the culture, the culture was conducted under the conditions of 30° C. and a relative dissolved oxygen concentration of 23% or more relative to the saturation concentration (air flow rate: ½ vvm, PL electrode reading of 5 or greater), until an OD620 of 38 was reached. When OD620 reached 38 (at 17.5 hours of cultivation), Tween 40 was added to a final concentration of 1 g/L, and the culture conditions were set to four different conditions: with or without oxygen limitation and with or without temperature elevation (changing the culture temperature from 30° C. to 35° C.), and cultivation was continued. The “no limitation” condition indicates that neither oxygen limitation nor temperature elevation was applied. The “temperature elevation only” condition refers to a condition in which oxygen limitation was not implemented, but temperature elevation was implemented. The “oxygen limitation only” condition refers to a condition in which temperature elevation was not implemented, but oxygen limitation was implemented. The “temperature elevation×oxygen limitation” condition refers to a condition in which both oxygen limitation and temperature elevation were implemented. In cases where oxygen limitation was implemented, this was achieved by lowering the agitation speed in the jar fermentor, inducing the PL electrode reading close to zero. Sampling was performed over time during the culture.

For the PL electrode, zero-point calibration was conducted without connecting the sensor to the electrode (electromotive force zero), and two-point calibration was performed by measuring the dissolved oxygen concentration of the culture medium saturated with dissolved oxygen by aeration and agitation at 30° C. with the PL electrode reading set to 21 (Same procedure was used in subsequent embodiments). Since the saturated dissolved oxygen concentration at 30° C. (atmospheric pressure 760 mmHg, oxygen 20.9%, saturated with water vapor) is 7.53 ppm, a PL electrode reading of 5 at 30° C. indicates a dissolved oxygen concentration of approximately 1.8 ppm (=7.53 ppm× 5/21). In addition, since the saturated dissolved oxygen concentration at 35° C. (atmospheric pressure 760 mmHg, oxygen 20.9%, saturated with water vapor) is 7.04 ppm, and the PL display value at that time was 24.3 (determined separately), a PL electrode reading of 5 at 35° C. indicates a dissolved oxygen concentration of approximately 1.46 ppm (=7.04×5/24.3).

Thus, four types of time-course culture broth samples containing L-glutamic acid were obtained. The amount of L-glutamic acid accumulated in the culture medium and the amount of glucose in the culture medium were measured using a biosensor BF-7 (Oji Scientific Instruments Co., Ltd.). The amount of organic acids accumulated in the medium was measured by liquid chromatography.

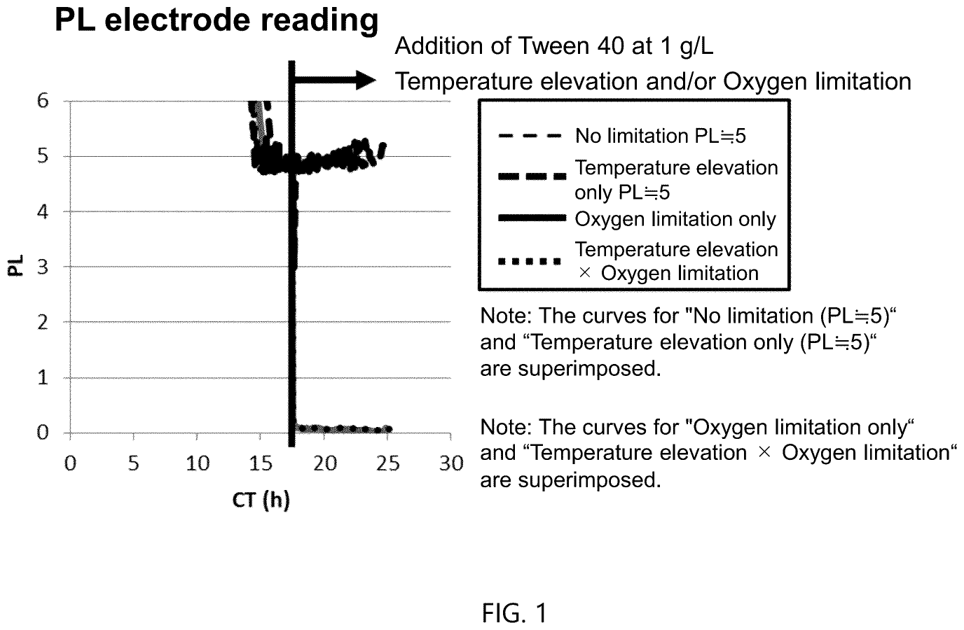

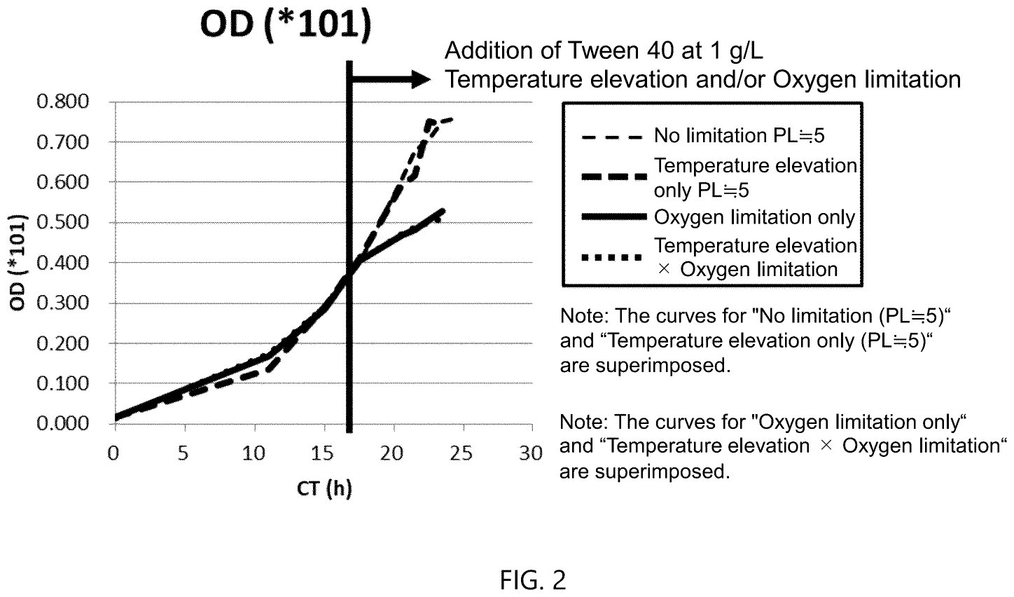

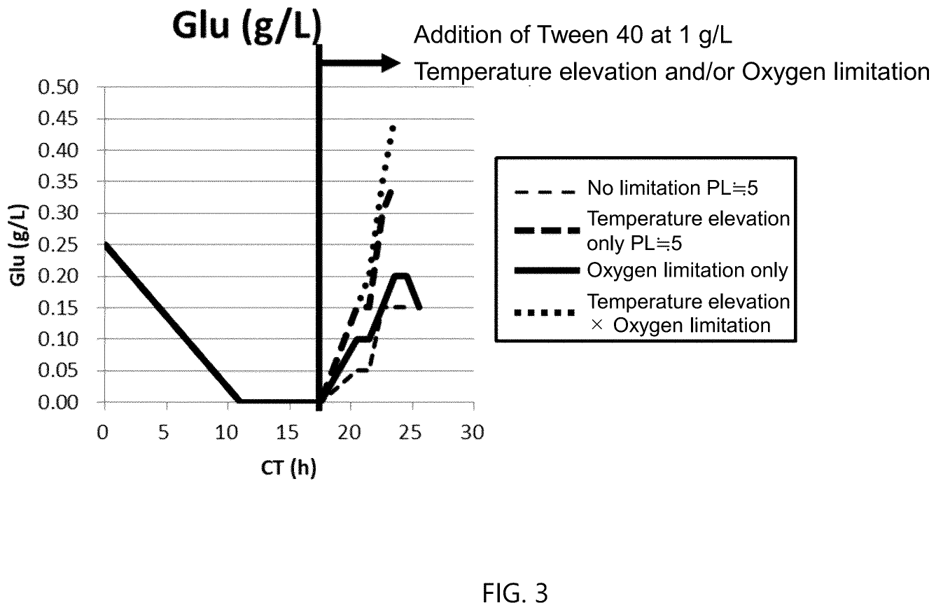

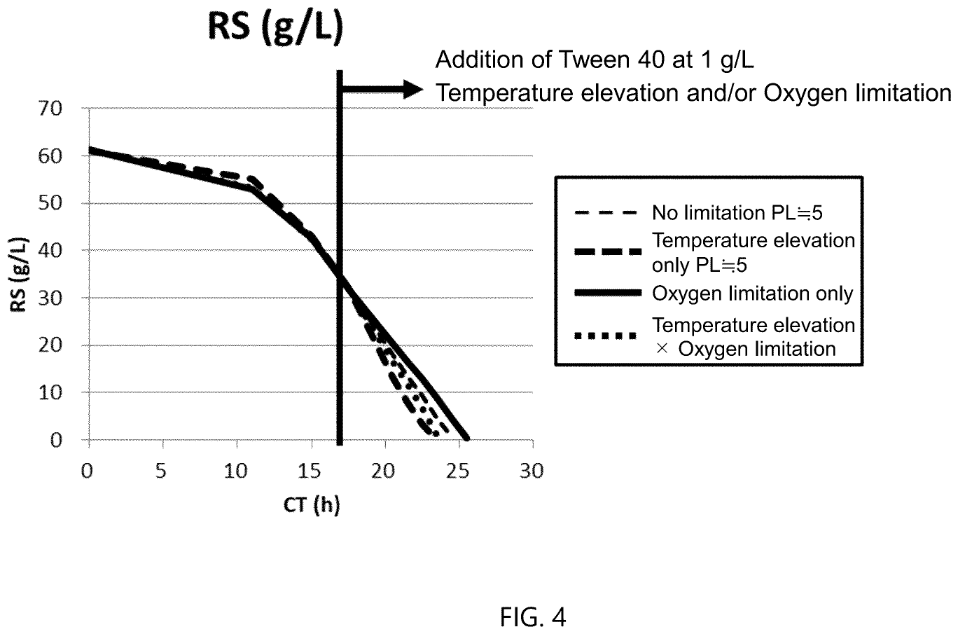

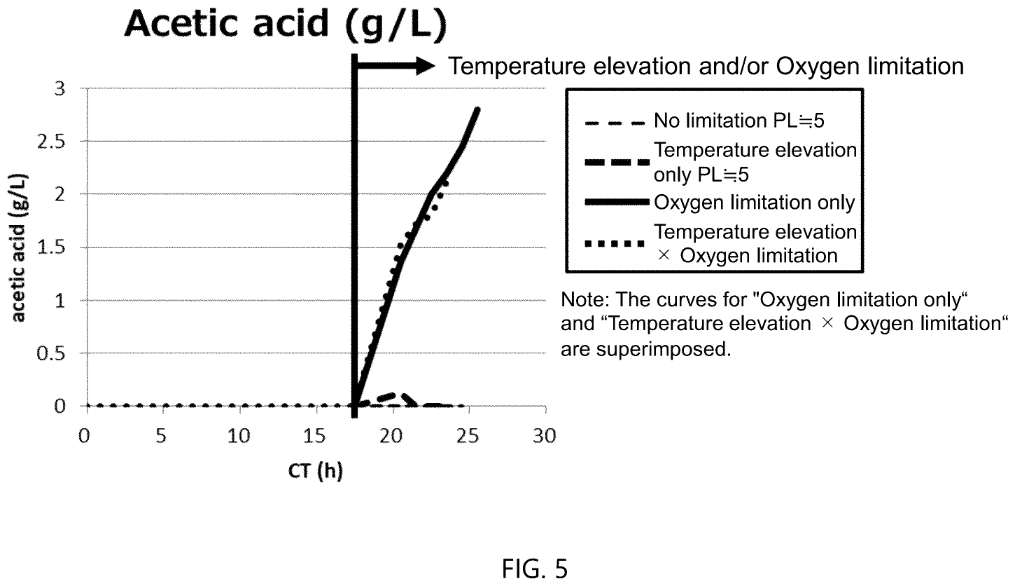

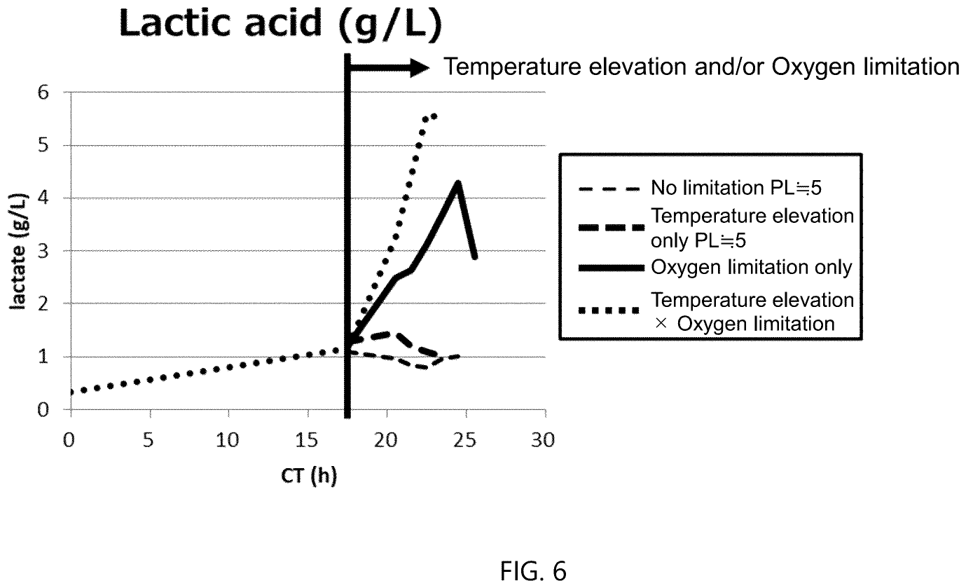

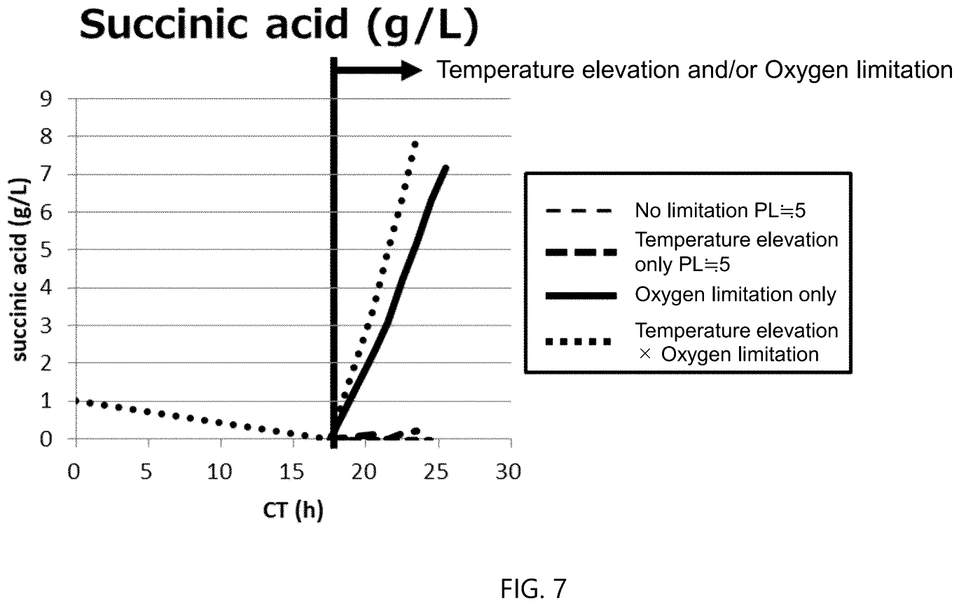

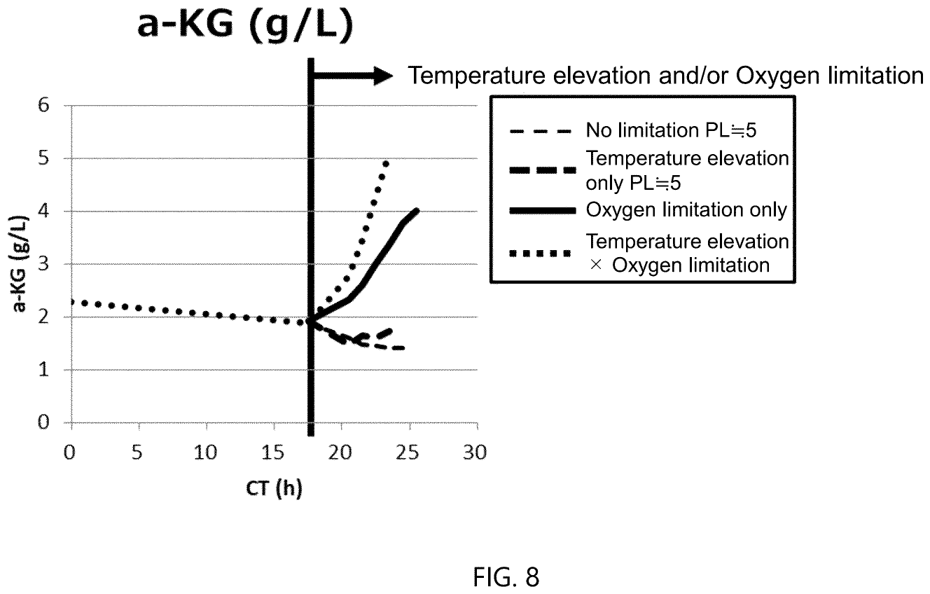

The results are shown in Table 5 and FIGS. 1 to 8. In C. casei JCM 12072 (wild-type strain), compared with the “no limitation” condition, Glu production increased under either the “temperature elevation only” or “oxygen limitation only” conditions. Furthermore, in C. casei JCM 12072 (wild-type strain), a marked increase in Glu production was observed under the “temperature elevation×oxygen limitation” condition. Additionally, under oxygen limitation conditions (“oxygen limitation only” and “temperature elevation×oxygen limitation” conditions), increased accumulation of organic acids and decreased cell biomass were observed compared to the “no limitation” condition.

Generally, in cultures under oxygen limitation conditions, respiratory efficiency decreases, resulting in a tendency for decreased efficiency of amino acid production around the TCA cycle, including Glu, and increased accumulation of organic acids. In addition, in general, in cultures under oxygen limitation conditions, ATP production, the energy currency of the cell, is suppressed, resulting in reduced cell proliferation. In the culture using C. casei JCM 12072 (wild-type strain) in this example as well, an increase in accumulation of organic acids and a decrease in cell growth were noted under oxygen limitation conditions. Similarly, in cultures using C. glutamicum, which is the most typical L-glutamic acid-producing bacterium, in Examples 6 and 7 described below, increased accumulation of organic acids and decreased cell growth were also confirmed under oxygen limitation conditions. In contrast, Glu production increased under oxygen limitation conditions in the culture using C. casei JCM 12072 (wild-type strain) in this example, whereas Glu production decreased under oxygen limitation conditions in cultures using C. glutamicum in Examples 6 and 7 described below. Furthermore, in the culture using C. casei JCM 12072 (wild-type strain) in this example, a remarkable increase in Glu production was observed under combined oxygen limitation and temperature elevation conditions—a result significantly different from general microbial metabolism or the result in C. glutamicum, and thus was not easily predictable. In other words, this example demonstrated that oxygen limitation is effective for L-glutamic acid production in C. casei, and the combination of oxygen limitation and temperature elevation is particularly effective. Furthermore, in Examples 3 to 5 described below, in various mutant strains of C. casei with improved L-glutamic acid-producing ability, a more pronounced effect of improved Glu production by oxygen limitation or the combination of oxygen limitation and temperature elevation was observed.

TABLE 5

| Genus and | Corynebacterium casei | | | |
|---|---|---|---|---|
| Species | | | | |
| Strain | JCM 12072 strain (WT) | | | |
| | | | | Temperature |
| | | Temperature | Oxygen | elevation |
| Condition or | No | elevation | limitation | Oxygen × |
| Purpose | limitation | only | only | limitation |
| Symbol | [symbol] | [symbol] | [symbol] | [symbol] |
| Yield (%) | 0.24 | 0.56 | 0.25 | 0.71 |
| BO-Glu conc. | 0.15 | 0.35 | 0.15 | 0.45 |
| (g/L) | | | | |
| Main CT (h) | 22.8 | 21.7 | 26.3 | 23.5 |

TABLE 6

| Component | Final Concentration | |
|---|---|---|
| Glucose | 45 | g/L |
| KH₂PO₄ | 3.52 | g/L |
| MgSO₄•7H₂O | 0.45 | g/L |
| FeSO₄•7H₂O | 10 | mg/L |
| Bean Concentrated | 1540 | mg/L |
| 2Na succinate | 2 | g/L |
| L (+) Ascorbic Acid | 8.55 | mg/L |
| VB1.HCl | 23 | mg/L |
| VB12 | 4 | μg/L |
| Biotin | 3.2 | mg/L |
| Yeast Extract | 5 | g/L |
| Defoamer (PP-AJ-2K) | 0.1 | mL/L |

TABLE 7

| Component | Final Concentration | |
|---|---|---|
| Glucose | 60 | g/L |
| KH₂PO₄ | 3.46 | g/L |
| MgSO₄•7H₂O | 0.5 | g/L |
| FeSO₄•7H₂O | 10 | mg/L |
| Bean Concentrated | 500 | mg/L |
| 2Na succinate | 2 | g/L |
| L (+) Ascorbic Acid | 8.55 | mg/L |
| VB1.HCl | 23 | mg/L |
| VB12 | 4 | μg/L |
| Biotin | 0.5 | mg/L |
| Defoamer (PP-AJ-2K) | 0.2 | mL/L |
| Betaine | 0.6 | g/L |

### Example 3: Evaluation of the Impact of Increased Culture Temperature and/or Oxygen Limitation on Glu Production During Culture of C. casei AJ111891 (NITE BP-03688)

The impact of increased culture temperature and/or oxygen limitation on Glu production was evaluated in C. casei AJ111891, a mutant strain with enhanced Glu-producing ability derived from C. casei JCM 12072 (wild-type strain). The increase in culture temperature is also referred to simply as “temperature elevation”.

(3-1) Evaluation of the Impact of Increased Culture Temperature and/or Oxygen Limitation on Glu Production During Culture of C. casei AJ111891

First, seed culture of AJ111891 strain was performed via jar fermentation using the seed culture medium shown in Table 6. The culture temperature was maintained constantly at 30° C., and the culture pH was controlled at 6.8 with ammonia gas.

Subsequently, the main culture of AJ111891 strain was performed via jar fermentation using the culture medium described in Table 7. The culture pH was maintained at 6.8 using ammonia gas, as with the seed culture. After inoculation with 15% seed culture broth to start the culture, the culture was conducted under the conditions of 30° C. and a relative dissolved oxygen concentration of 23% or more relative to the saturation concentration (air flow rate: ½ vvm, PL electrode reading of 5 or greater), until an OD620 of 40 was reached. When OD620 reached 40 (at 10 hours of cultivation), the culture conditions were set to four different conditions: with or without oxygen limitation and with or without temperature elevation (changing the culture temperature from 30° C. to 35° C.), and cultivation was continued. The “no limitation” condition indicates that neither oxygen limitation nor temperature elevation was applied. The “temperature elevation only” condition refers to a condition in which oxygen limitation was not implemented, but temperature elevation was implemented. The “oxygen limitation only” condition refers to a condition in which temperature elevation was not implemented, but oxygen limitation was implemented. The “temperature elevation×oxygen limitation” condition refers to a condition in which both oxygen limitation and temperature elevation were implemented. In cases where oxygen limitation was implemented, this was achieved by lowering the agitation speed in the jar fermentor, inducing the PL electrode reading close to zero. In cases where oxygen limitation was not implemented, this was achieved by maintaining the agitation speed in the jar fermentor in a higher range than in the oxygen limitation condition, inducing the PL electrode reading close to 0.5. Sampling was performed over time during the culture.

Thus, four types of time-course culture broth samples containing L-glutamic acid were obtained. The amount of L-glutamic acid accumulated in the culture medium and the amount of glucose in the culture medium were measured using a biosensor BF-7 (Oji Scientific Instruments Co., Ltd.). The amount of organic acids accumulated in the medium was measured by liquid chromatography.

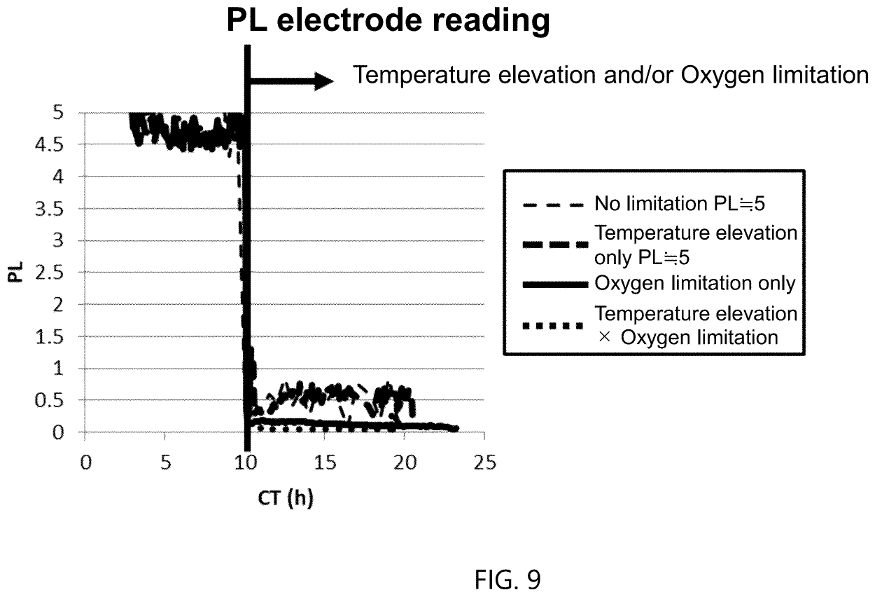

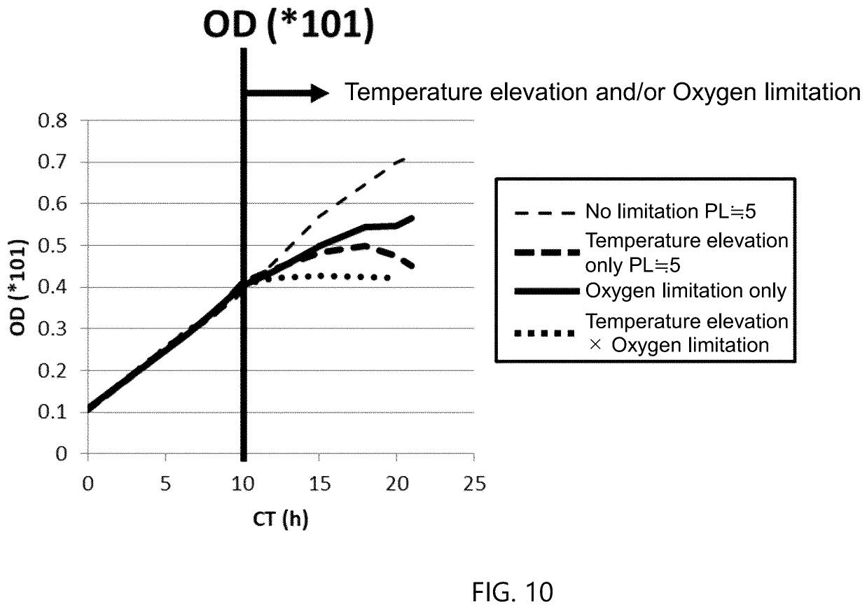

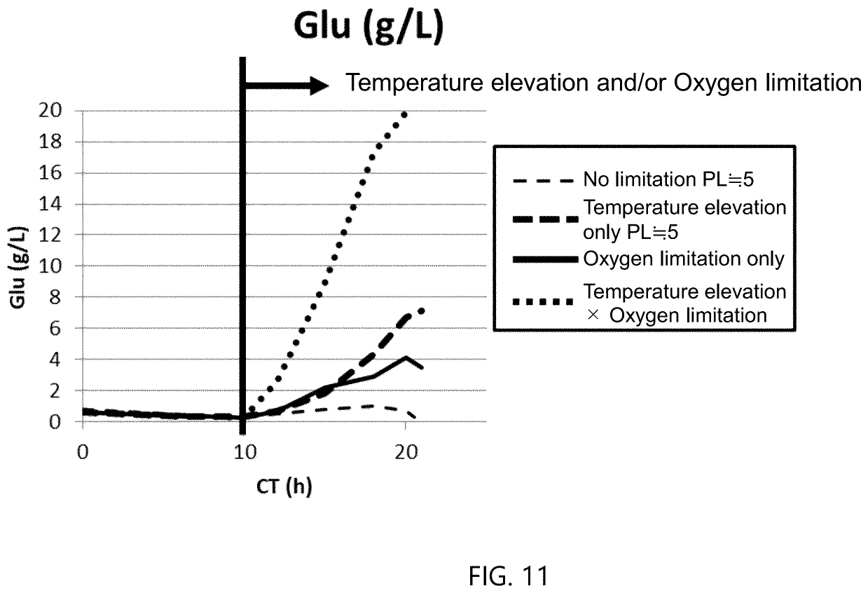

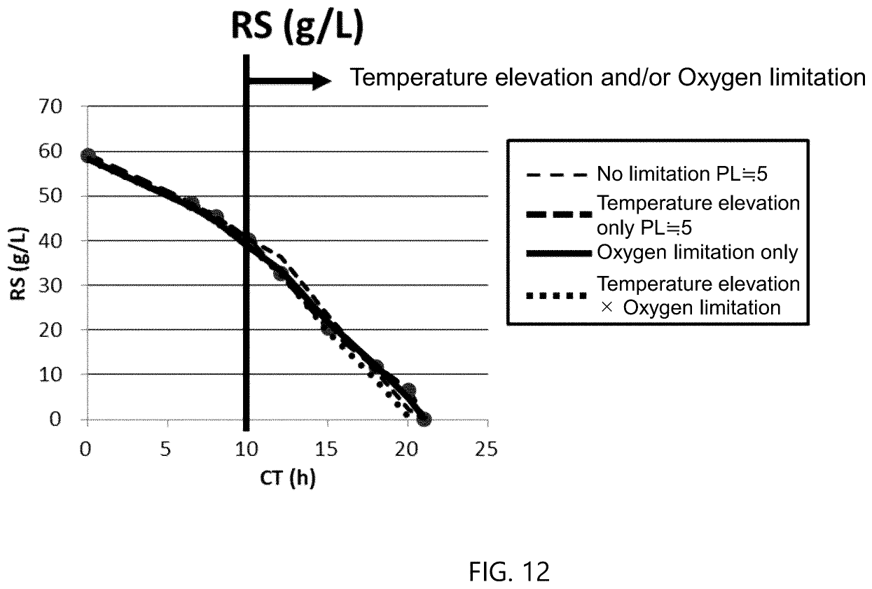

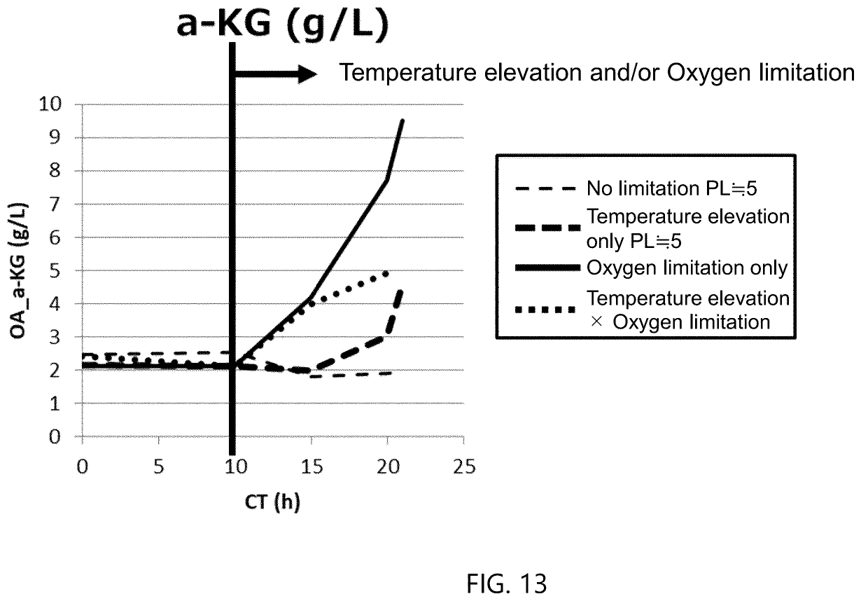

The results are shown in Table 8 and FIGS. 9 to 13. In C. casei AJ111891, Glu production was hardly observed under the “no limitation” condition. In contrast, under the “temperature elevation only” or “oxygen limitation only” condition, Glu production increased. Furthermore, in C. casei AJ111891, under the “temperature elevation×oxygen limitation” condition, Glu production increased significantly. Furthermore, under oxygen limitation conditions (“oxygen limitation only” and “temperature elevation×oxygen limitation” conditions), as compared to the “no limitation” condition, an increase in the accumulation of organic acids and a decrease in cell mass were confirmed.

This example further demonstrates that, in C. casei, oxygen limitation is effective for Glu production, and the combination of oxygen limitation and temperature elevation is particularly effective. Moreover, it was demonstrated that in the mutant strain of C. casei with enhanced Glu-producing ability compared to JCM 12072 (wild-type strain), a more significant increase in Glu production can be obtained by oxygen limitation or by the combination of oxygen limitation and temperature elevation.

Separately, it was confirmed that the growth and Glu production of C. casei AJ111891 were comparable under the conditions with a PL electrode reading of 5 or greater and under the conditions with a PL electrode reading of around 0.5.

TABLE 8

| Genus and | Corynebacterium casei | | | |
|---|---|---|---|---|
| Species | | | | |
| Strain | AJ111891 | | | |
| | | | | Temperature |
| | | Temperature | Oxygen | elevation × |
| Condition | No | elevation | limitation | Oxygen |
| or Purpose | limitation | only | only | limitation |
| Symbol | [symbol] | [symbol] | [symbol] | [symbol] |
| Yield (%) | 1.5 | 14.4 | 6.0 | 34.6 |
| BO-Glu conc. (g/L) | 0.9 | 8.9 | 3.5 | 19.9 |
| Main CT (h) | 20.6 | 22.6 | 21.0 | 20.2 |

(3-2) Evaluation of the Impact of the Degree of Oxygen Limitation on Glu Production During Culture of C. casei AJ111891

Examples 2 and 3 (3-1) demonstrated that implementing oxygen limitation so that the PL electrode reading approaches zero is effective for the production of L-glutamic acid by C. casei. Next, the impacts on L-glutamic acid production by C. casei when varying the degree of oxygen limitation, and the corresponding impact on time-course L-glutamic acid production efficiency, were evaluated.

First, seed culture of AJ111891 strain was performed via jar fermentation using the seed culture medium shown in Table 6. The culture temperature was maintained constantly at 30° C., and the culture pH was controlled at 6.8 with ammonia gas.

Subsequently, the main culture of the AJ111891 strain was performed via jar fermentation using the culture medium described in Table 7. The culture pH was maintained at 6.8 using ammonia gas, as with the seed culture. Until OD620 reached 40, the culture temperature was maintained at 30° C., and the dissolved oxygen concentration was maintained at a value of 23% or more relative to the saturation concentration (aeration rate: ½ vvm, PL electrode reading: 5 or greater). Then, upon OD620 reaching 40 (10 hours of culture), the culture conditions were established as either: oxygen limitation only, or a combination of oxygen limitation and increased temperature (elevating the culture temperature from 30° C. to 35° C.), and culture was continued. When implementing oxygen limitation, the agitation speed of the jar fermenter was lowered, thereby inducing the PL electrode reading to close to zero. At this time, three agitation speeds (370 rpm, 440 rpm, 550 rpm) were set to induce three types of oxygen limitation conditions differing in oxygen supply. In the condition of oxygen limitation only, the Rab value was approximately 4 at 370 rpm, 5 to 7 at 440 rpm, and 7 to 9 at 550 rpm. In the condition of oxygen limitation only, the Rab value was approximately 4 at 370 rpm, 6 to 7 at 440 rpm, and 8 to 9 at 550 rpm. Under each oxygen limitation condition, since the PL electrode reading is close to zero, it can be considered that the oxygen supply is nearly balanced with the oxygen consumption rate (Rab) calculated from the oxygen concentration in the inlet and outlet gases.

Thus, six types of time-course culture broth samples containing L-glutamic acid were obtained. The amount of L-glutamic acid accumulated in the culture medium and the amount of glucose in the culture medium were measured using a biosensor BF-7 (Oji Scientific Instruments Co., Ltd.).

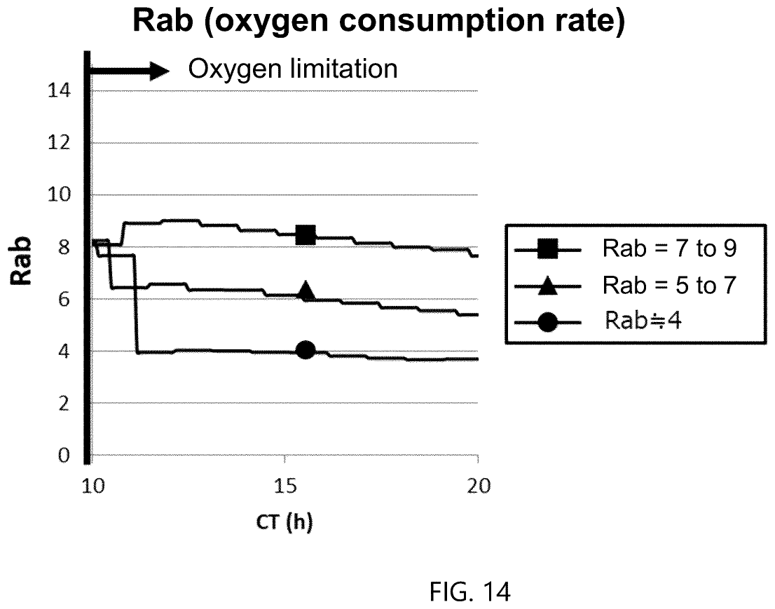

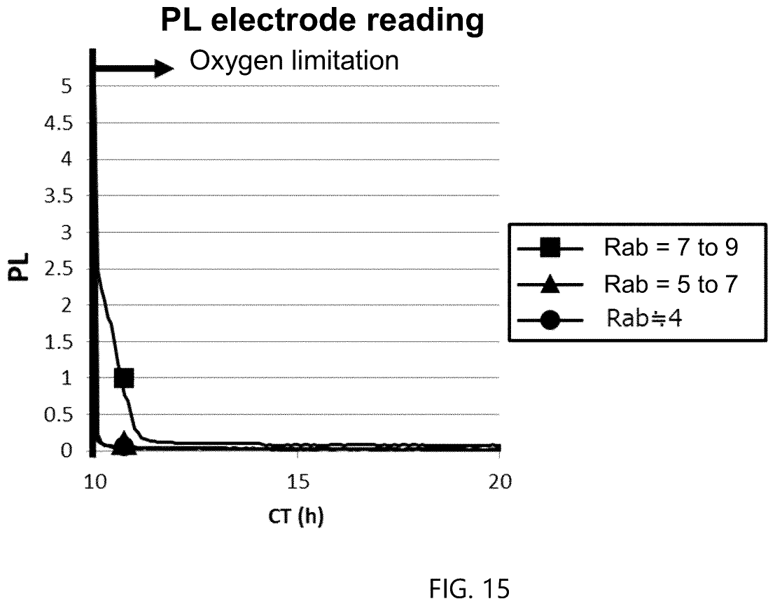

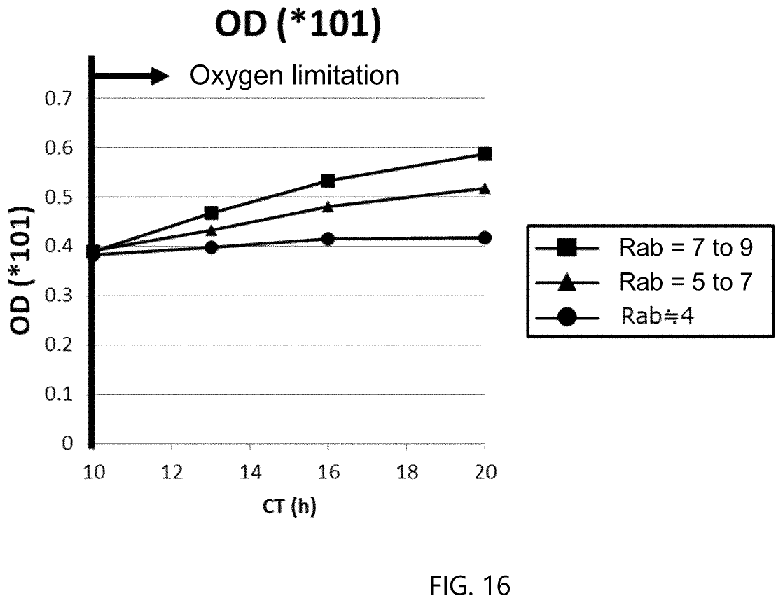

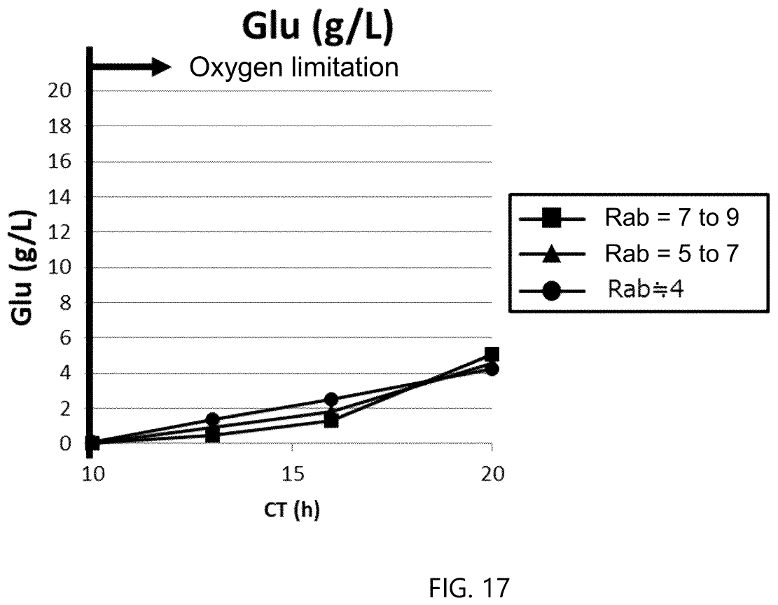

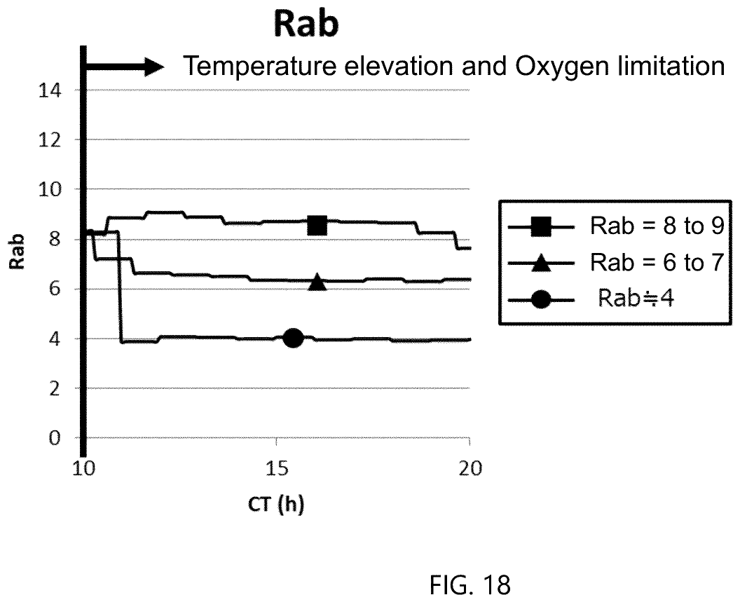

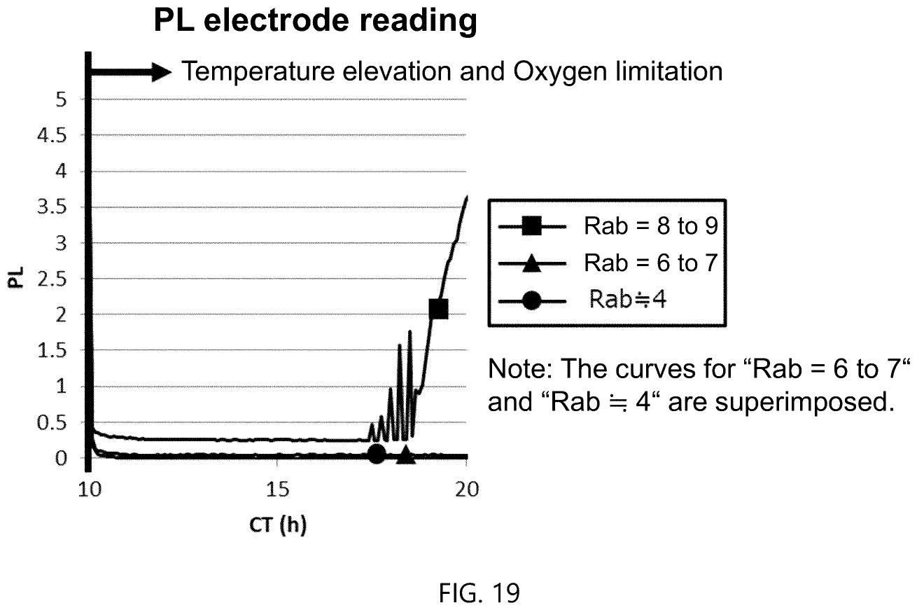

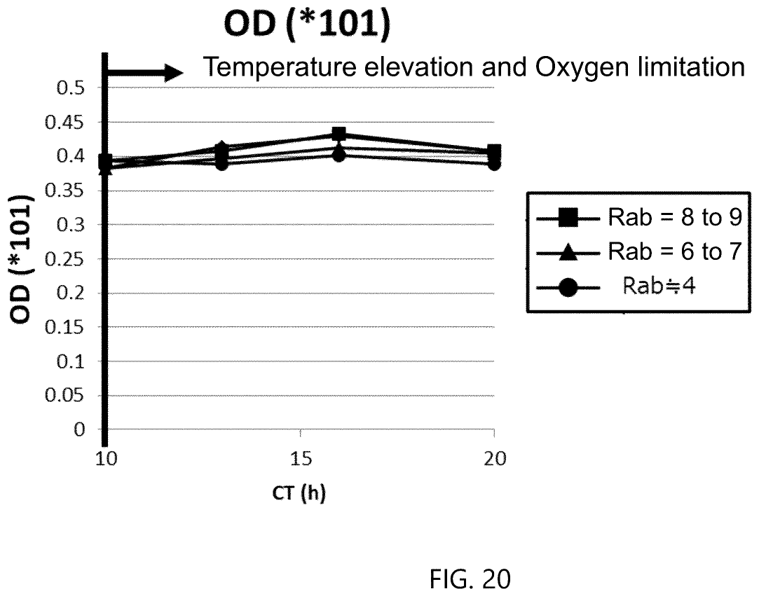

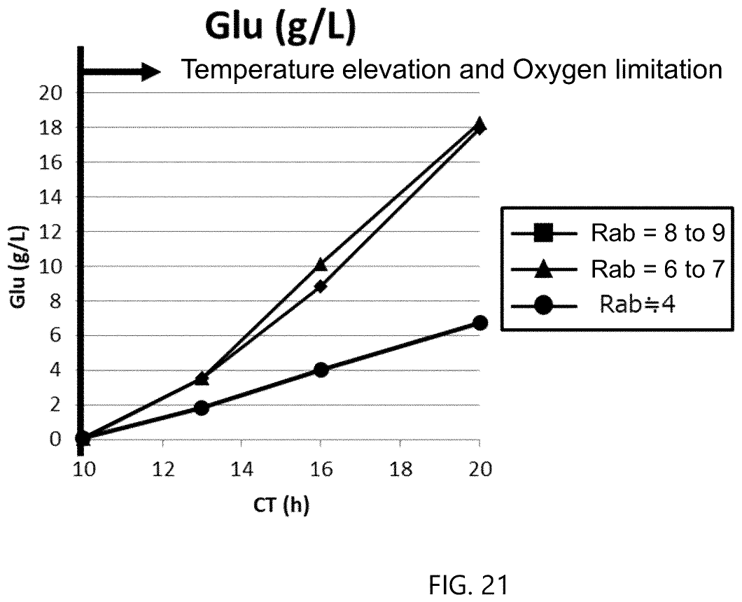

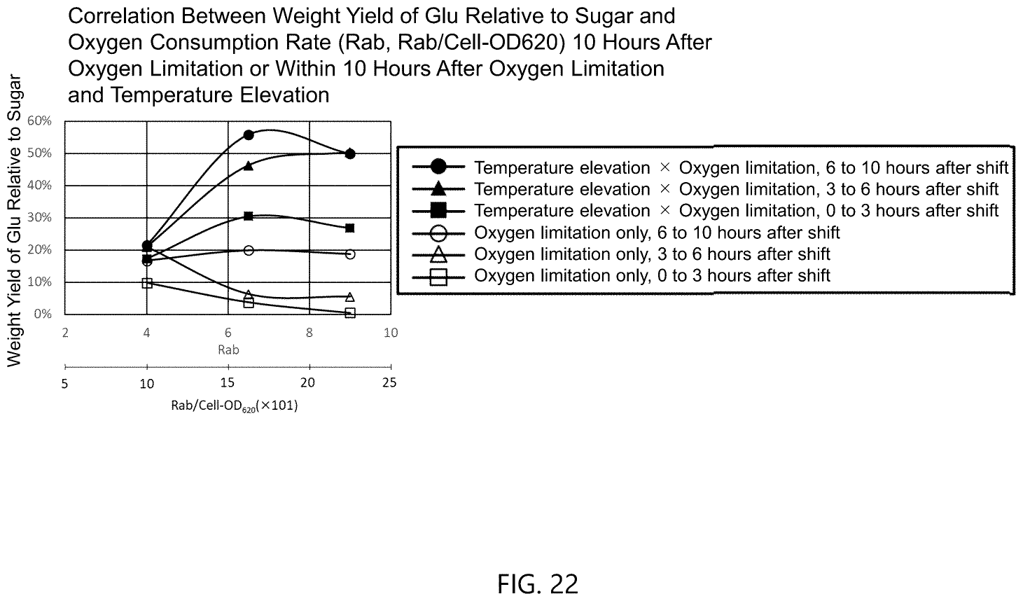

The results obtained under the condition of oxygen limitation only are shown in FIGS. 14 to 17. The results obtained under the condition of oxygen limitation and increased temperature are shown in FIGS. 18 to 21. The correlation between weight yield of Glu relative to sugar and the oxygen consumption rate (Rab or Rab/Cell-OD620) is shown in FIG. 22. Note that in FIG. 22, the Rab/Cell-OD620 value was uniformly calculated based on OD620 (×10¹)=0.4 at the time of implementing oxygen limitation. However, the actual Rab/Cell-OD620 value may vary depending on whether and to what extent cell proliferation occurs after initiation of oxygen limitation. In other words, for example, the actual Rab/Cell-OD620 value under the conditions where cell proliferation occurred after initiation of oxygen limitation will be smaller than the value shown in FIG. 22. In the case of C. casei AJ111891, efficient L-glutamic acid production was observed at any degree of oxygen limitation. Moreover, in the case of C. casei AJ111891, it was shown that the efficiency of L-glutamic acid production differs depending on the degree of oxygen limitation. Specifically, an increasing trend in the yield of L-glutamic acid was observed when the Rab value was 4 or more. Also, there was a trend towards improved L-glutamic acid production efficiency when the Rab/Cell-OD620 (×10¹) value was 10 or more. Thus, it was shown that, for L-glutamic acid production by C. casei, it is effective to perform oxygen limitation while maintaining the oxygen consumption rate at or above a certain threshold (for example, Rab value of 4 or more, or Rab/Cell-OD620 (×10¹) value of 10 or more), by, for example, inducing the PL electrode reading to close to zero.

In addition, after setting the culture conditions as described above, it was observed that, while the amount of cells did not increase significantly, the L-glutamic acid yield increased over time. From this, it was suggested that metabolic fluctuations, including gene expression, within the cells are induced by oxygen limitation or the combination of oxygen limitation and increased temperature. The metabolic fluctuations caused by gene expression are supported by the fact that, under the condition of “increased temperature×oxygen limitation” with Rab=8-9, a high sugar-to-L-glutamic acid yield was maintained even after the PL electrode reading increased at the end of culture. In other words, even if the culture conditions are returned to their original state (e.g., a culture temperature of 30° C. or aerobic conditions) after a predetermined period for which gene expression can be induced by oxygen limitation or the combination of oxygen limitation and increased temperature, high L-glutamic acid production can be maintained to some extent.

### Example 4: Evaluation of the Impact of Increased Culture Temperature and/or Oxygen Limitation on L-Glutamic Acid Production During Culture of C. casei A-013 (NITE BP-03806)

The impact of increased culture temperature and/or oxygen limitation on L-glutamic acid production was evaluated in C. casei A-013, a mutant strain with enhanced L-glutamic acid producing ability derived from C. casei JCM 12072 (wild-type strain). The increase of culture temperature is referred to simply as “temperature elevation”.

First, seed culture of A-013 strain was performed via jar fermentation using the seed culture medium shown in Table 6. The culture temperature was maintained constantly at 30° C., and the culture pH was controlled at 6.8 with ammonia gas.

Subsequently, the main culture of A-013 strain was performed via jar fermentation using the culture medium described in Table 7. The culture pH was maintained at 6.8 using ammonia gas, as with the seed culture. After inoculation with 15% seed culture broth to start the culture, the culture was conducted under the conditions of 30° C. and a relative dissolved oxygen concentration of 23% or more (air flow rate: ½ vvm, PL electrode reading of 5 or greater), until an OD620 of 40 was reached. When OD620 reached 40 (at 8.5 hours of cultivation), the culture conditions were set to four different conditions: with or without oxygen limitation and with or without temperature elevation (changing the culture temperature from 30° C. to 35° C.), and cultivation was continued. The “no limitation” condition indicates that neither oxygen limitation nor temperature elevation was applied. The “temperature elevation only” condition refers to a condition in which oxygen limitation was not implemented, but temperature elevation was implemented. The “oxygen limitation only” condition refers to a condition in which temperature elevation was not implemented, but oxygen limitation was implemented. The “temperature elevation×oxygen limitation” condition refers to a condition in which both oxygen limitation and temperature elevation were implemented. When implementing oxygen limitation, the agitation speed of the jar fermenter was lowered, thereby inducing the PL electrode reading to close to zero. Sampling was performed over time during the culture.

Thus, four types of time-course culture broth samples containing L-glutamic acid were obtained. The amount of L-glutamic acid accumulated in the culture medium and the amount of glucose in the culture medium were measured using a biosensor BF-7 (Oji Scientific Instruments Co., Ltd.). The amount of organic acids accumulated in the medium was measured by liquid chromatography.

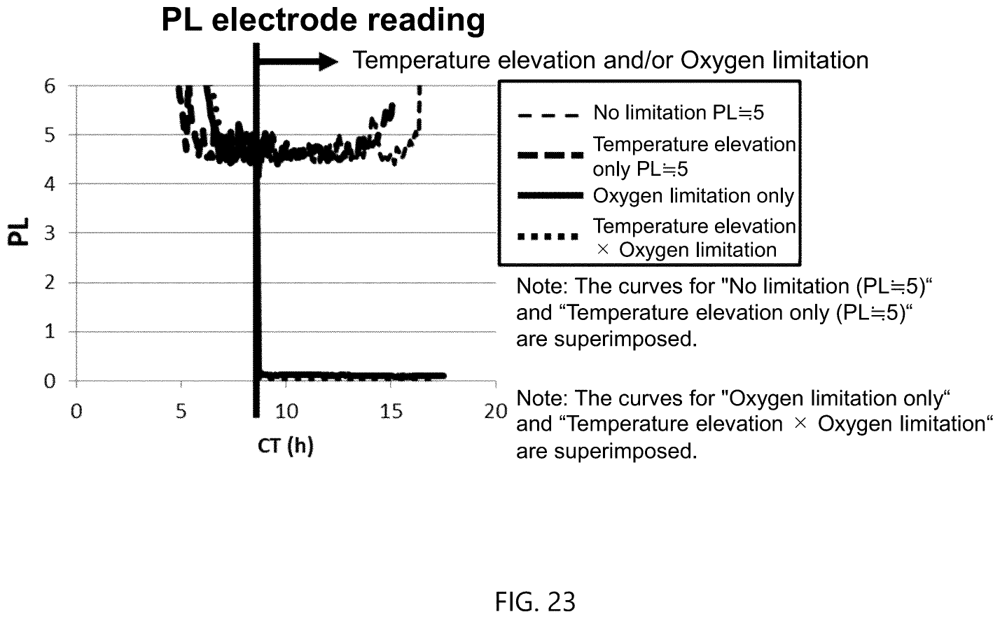

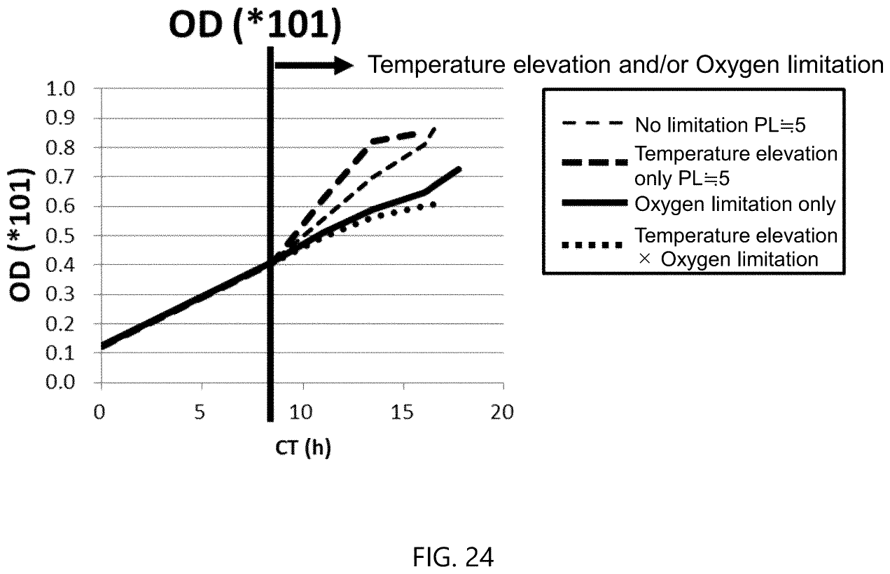

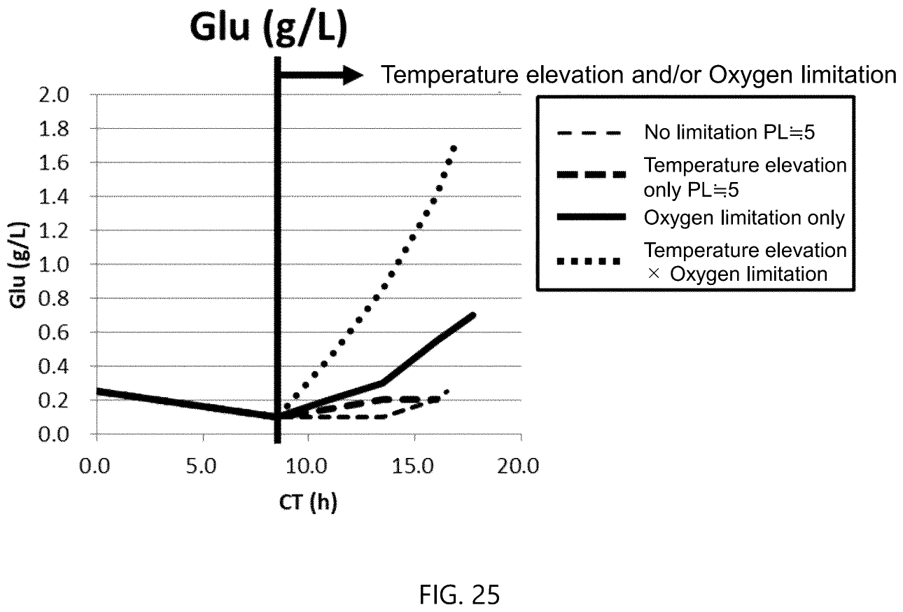

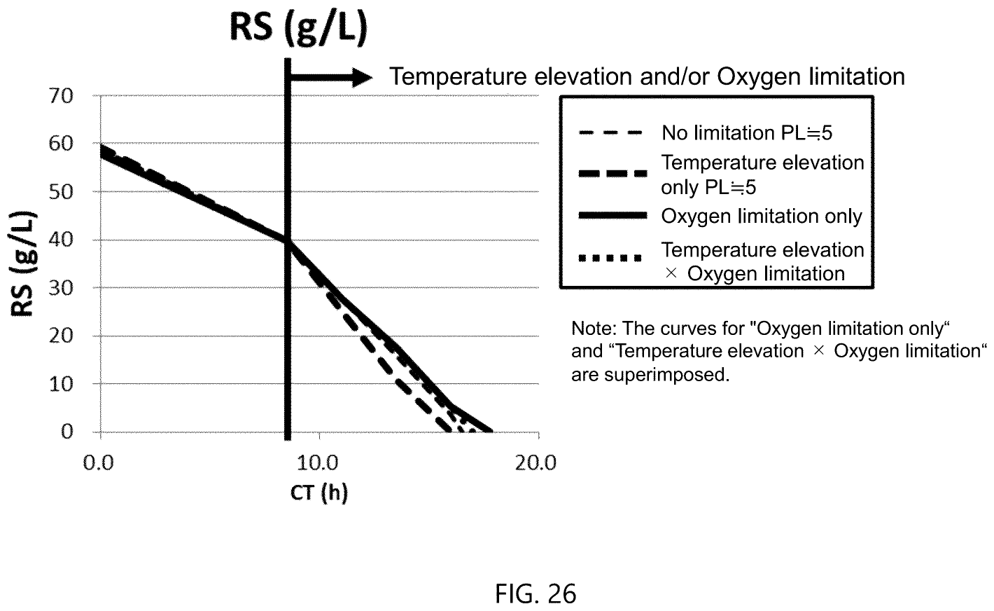

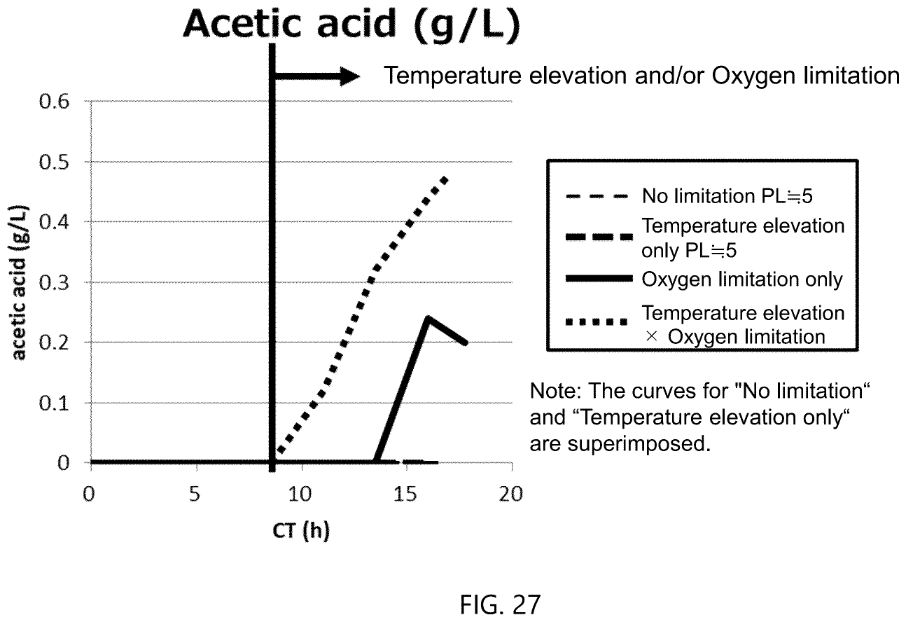

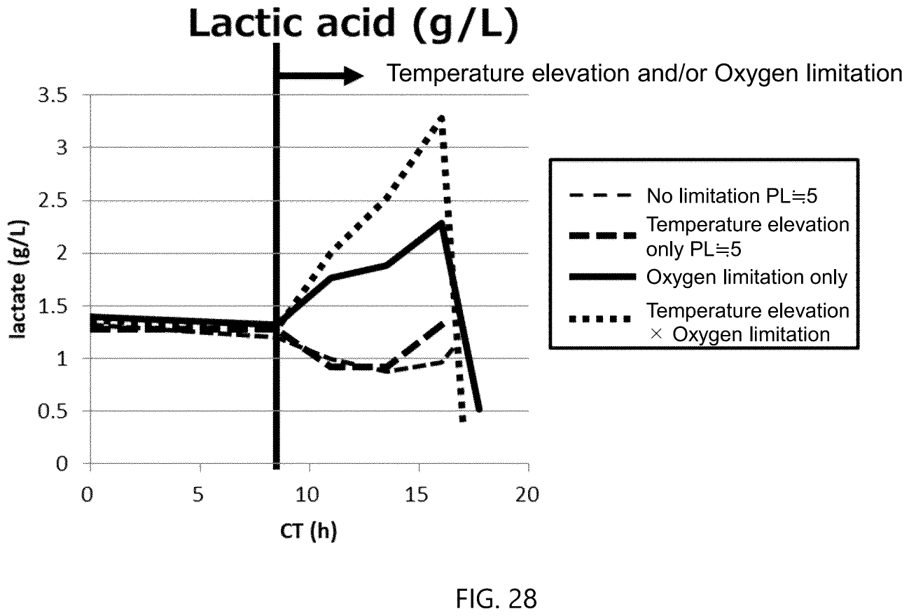

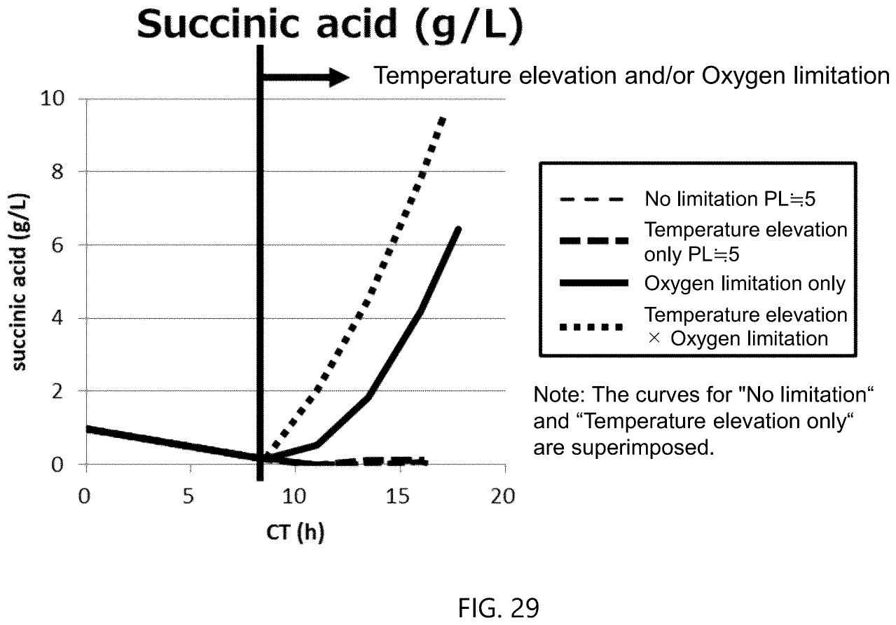

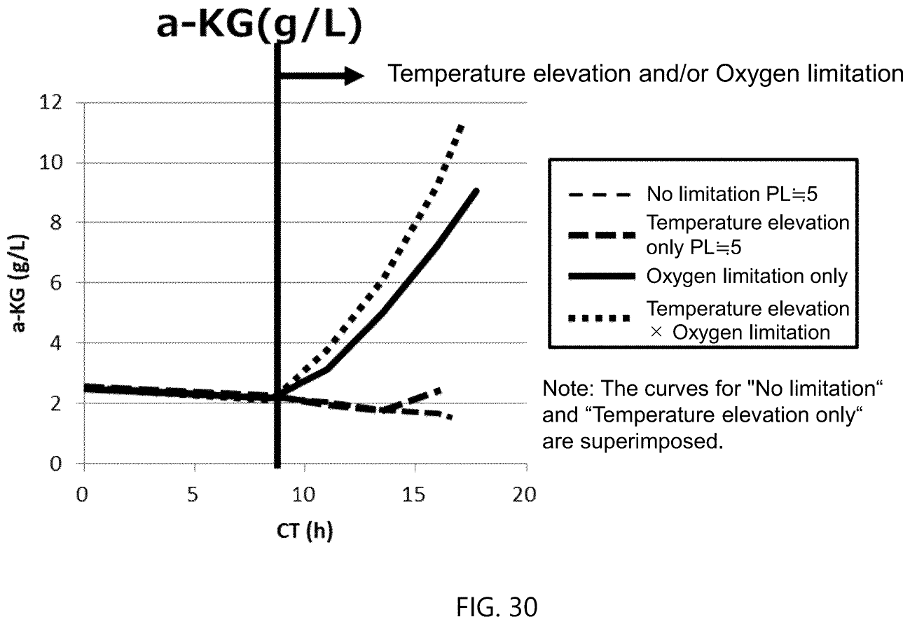

The results are shown in Table 9 and FIGS. 23 to 30. In C. casei A-013 strain, Glu production was hardly observed under the “no limitation” condition. In contrast, under the “oxygen limitation only” condition, Glu production increased. Furthermore, in C. casei A-013 strain, under the “temperature elevation×oxygen limitation” condition, Glu production increased significantly. Furthermore, under oxygen limitation conditions (“oxygen limitation only” and “temperature elevation×oxygen limitation” conditions), as compared to the “no limitation” condition, an increase in the accumulation of organic acids and a decrease in cell mass were confirmed.

This example further demonstrates that, in C. casei, oxygen limitation is effective for Glu production, and the combination of oxygen limitation and temperature elevation is particularly effective. Moreover, it was demonstrated that in the mutant strain of C. casei with enhanced Glu-producing ability compared to JCM 12072 (wild-type strain), a more significant increase in Glu production can be obtained by oxygen limitation or by the combination of oxygen limitation and temperature elevation.

TABLE 9

| Genus and | Corynebacterium casei | | | |
|---|---|---|---|---|
| Species | | | | |
| Strain | A-013 | | | |
| | | | | Temperature |
| | | Temperature | Oxygen | elevation × |
| Condition or | No | elevation | limitation | Oxygen |
| Purpose | limitation | only | only | limitation |
| Symbol | [symbol] | [symbol] | [symbol] | [symbol] |
| Yield (%) | 0.4 | 0.3 | 1.2 | 3.0 |
| BO-Glu conc. | 0.3 | 0.2 | 0.7 | 1.8 |
| (g/L) | | | | |
| Main CT (h) | 16.4 | 15.1 | 17.5 | 16.8 |

### Example 5: Evaluation of the Impact of Increased Culture Temperature and/or Oxygen Limitation on Glu Production During Culture of C. casei Strain with Enhanced Dicarboxylic Acid Exporter

It is known that dicarboxylic acid exporters play an important role in Glu fermentation. Therefore, using C. casei JCM 12072 (wild-type strain) as the parent strain, a recombinant strain was constructed in which the ybjL gene (WO2008/133161), encoding a heterologous dicarboxylic acid exporter, was expressed from a plasmid. The impacts of increased culture temperature and/or oxygen limitation on Glu production were evaluated. The increase in culture temperature is also referred to simply as “temperature elevation”.

(5-1) Cloning of the ybjL Gene into the pVK9 Plasmid

Using the genomic DNA of Pantoea ananatis 359 (FERM BP-6614) strain as a template, a fragment containing the ybjL gene was amplified by PCR using PrimeSTAR Max (manufactured by TaKaRa) and a combination of primers 1 and 2, as shown in Table 10. Subsequently, using the genomic DNA of C. glutamicum 2256 (ATCC 13869) as a template and a combination of primers 3 and 4, a fragment containing the PmsrA promoter was amplified by PCR using PrimeSTAR Max (manufactured by TaKaRa). These two fragments were cloned into pVK9 (WO2007/046389) treated with BamHI and PstI using the InFusion HD kit (manufactured by TaKaRa), to construct an expression plasmid of the ybjL gene. The constructed plasmid was designated as pVK9-ybjL. Pantoea ananatis 359 (P. ananatis AJ13355 strain) strain was originally deposited at the Life Science and Biotechnology Research Center, Agency of Industrial Science and Technology, Ministry of International Trade and Industry (currently NITE IPOD, Postal Code: 292-0818, Address: Room 120, 2-5-8 Kazusa-Kamatari, Kisarazu-shi, Chiba, Japan) on Feb. 19, 1998, under the deposit number FERM P-16644. The deposit was converted to an international deposit under the Budapest Treaty on Jan. 11, 1999, and assigned the deposit number FERM BP-6614.

TABLE 10

| SEQ | |
|---|---|
| ID NOS. | Nucleotide sequences |
| 1 | ATGTTGAATGTTAACATCGCAG |
| 2 | cggtacccggggatcTTATAAGGGCAATAACGGCCAG |
| 3 | Ccaagcttgcatgccatttgcgcctgcaacgtaggttg |
| 4 | GTTAACATTCAACATaacaggaatgttcctttcgaaaa |

(5-2) Introduction of pVK9-ybjL Plasmid into C. casei JCM 12072 (Wild-Type Strain)

First, the JCM 12072 was spread onto an LB plate medium and cultured overnight at 30° C. A loopful of the grown cells was inoculated into 4 mL of a commonly composed LB medium supplemented with 2.5 g/L glycine and 1 g/L Tween 80, and cultured at 30° C. for 3 hours. Next, ampicillin was added to a final concentration of 5 g/mL, and the culture was continued at 30° C. for 1 hour. After that, the cells were collected by centrifugation at 12,000 rpm for 5 minutes and washed with 1 mL of chilled 10% glycerol. This washing step was repeated two additional times. Subsequently, the cells were suspended in an appropriate amount of chilled 10% glycerol to prepare competent cells. The competent cells were mixed with the pVK9-ybjL plasmid, and the plasmid was introduced into the cells by electroporation.

Approximately 80 μL of the competent cells subjected to electroporation were supplemented with 1 mL of SOC medium (Takara), and recovery culture was performed in a 14 mL round tube at 30° C., 120 rpm, for 3 hours. The recovery culture was then spread onto a plate medium composed of a common LB medium supplemented with 2 g/L disodium succinate hexahydrate and 25 mg/L kanamycin, and cultured at 30° C. for 2 days. Colonies that appeared were purified to obtain strains harboring the pVK9-ybjL plasmid in the JCM 12072, which were designated as WT+pVK9-ybjL strains.

(5-3) Evaluation of the Impact of Increased Culture Temperature and/or Oxygen Limitation on Glu Production During Culture of C. casei WT+pVK9-ybjL Strain

First, seed culture of WT+pVK9-ybjL strain was performed via jar fermentation using the culture medium of the composition shown in Table 6, supplemented with 25 mg/L kanamycin. The culture temperature was maintained constantly at 30° C., and the culture pH was controlled at 6.8 with ammonia gas.

Subsequently, the main culture of WT+pVK9-ybjL strain was performed via jar fermentation using the culture medium of the composition shown in Table 7, supplemented with 25 mg/L kanamycin. The culture pH was maintained at 6.8 using ammonia gas, as with the seed culture. After inoculation with 15% seed culture broth to start the culture, the culture was conducted under the conditions of 30° C. and a relative dissolved oxygen concentration of 23% or more (air flow rate: ½ vvm, PL electrode reading of 5 or greater), until an OD620 of 35 was reached. When OD620 reached 35 (at 14 hours of cultivation), the culture conditions were set to four different conditions: with or without oxygen limitation and with or without temperature elevation (changing the culture temperature from 30° C. to 35° C., and further to 37° C. 4 hours later), and cultivation was continued. The “no limitation” condition indicates that neither oxygen limitation nor temperature elevation was applied. The “temperature elevation only” condition refers to a condition in which oxygen limitation was not implemented, but temperature elevation was implemented. The “oxygen limitation only” condition refers to a condition in which temperature elevation was not implemented, but oxygen limitation was implemented. The “temperature elevation×oxygen limitation” condition refers to a condition in which both oxygen limitation and temperature elevation were implemented. When implementing oxygen limitation, the agitation speed of the jar fermenter was lowered, thereby inducing the PL electrode reading to close to zero. Sampling was performed over time during the culture.

Thus, four types of time-course culture broth samples containing L-glutamic acid were obtained. The amount of L-glutamic acid accumulated in the culture medium and the amount of glucose in the culture medium were measured using a biosensor BF-7 (Oji Scientific Instruments Co., Ltd.). The amount of organic acids accumulated in the medium was measured by liquid chromatography.

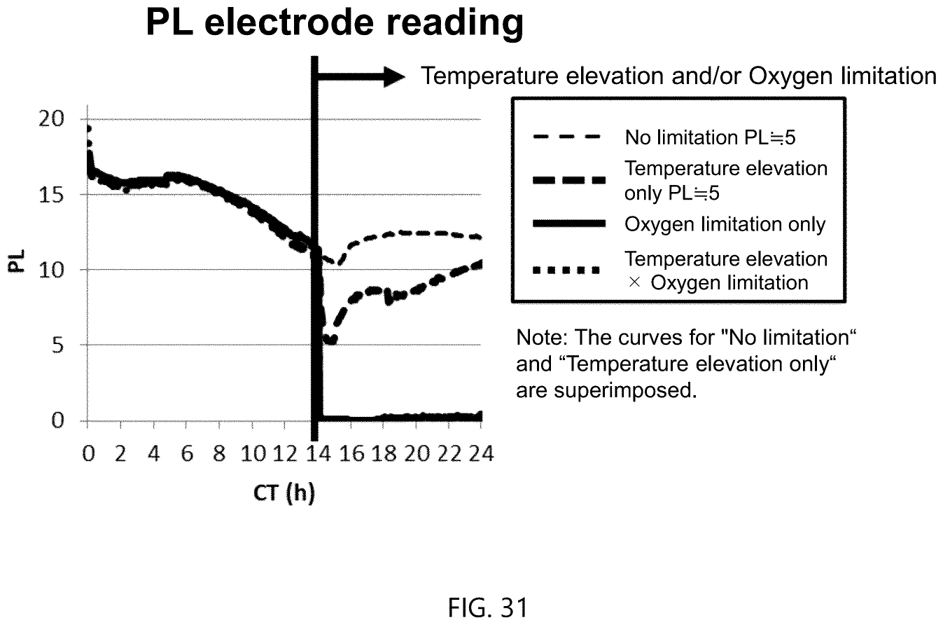

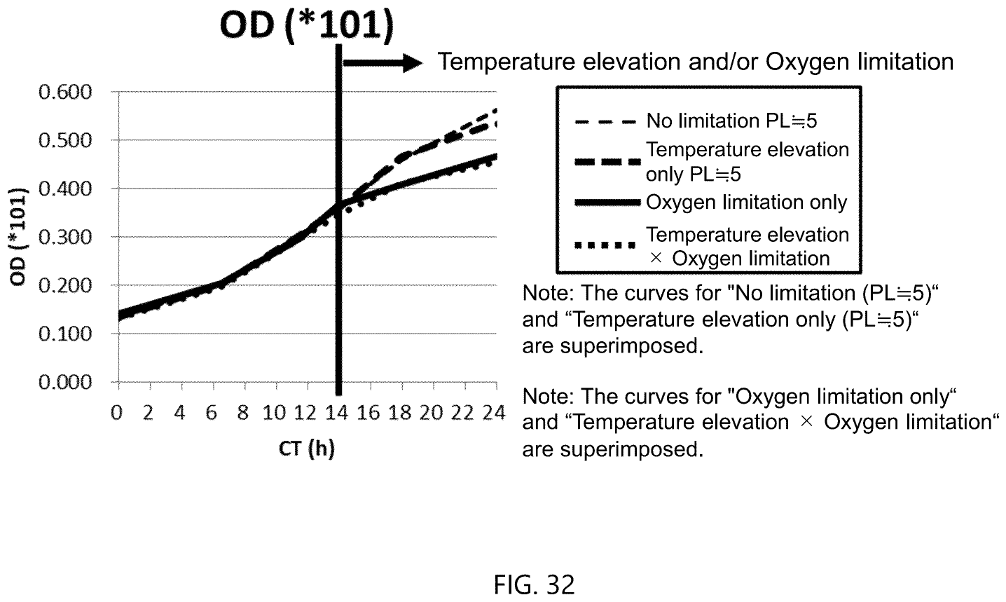

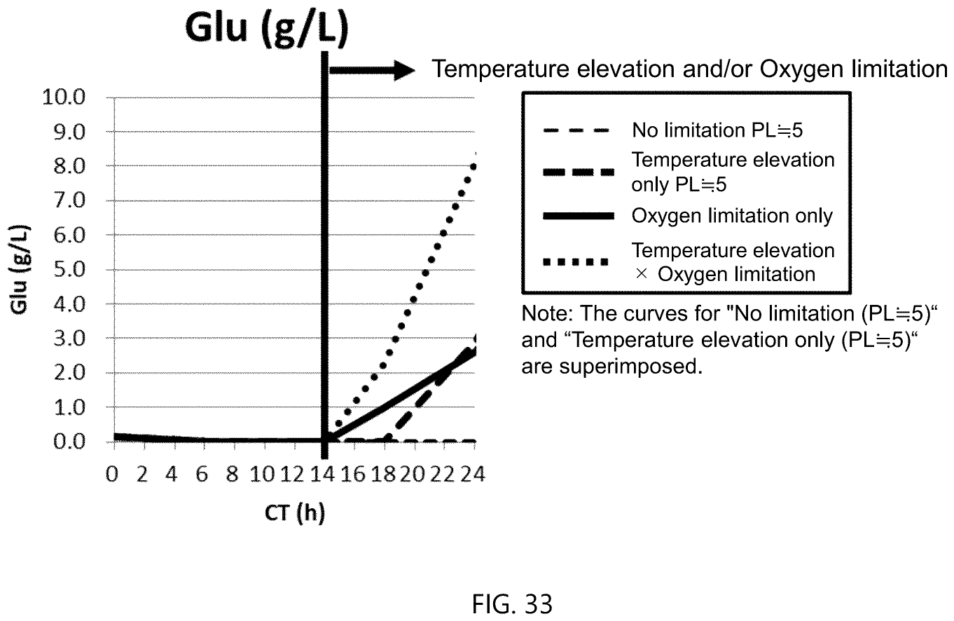

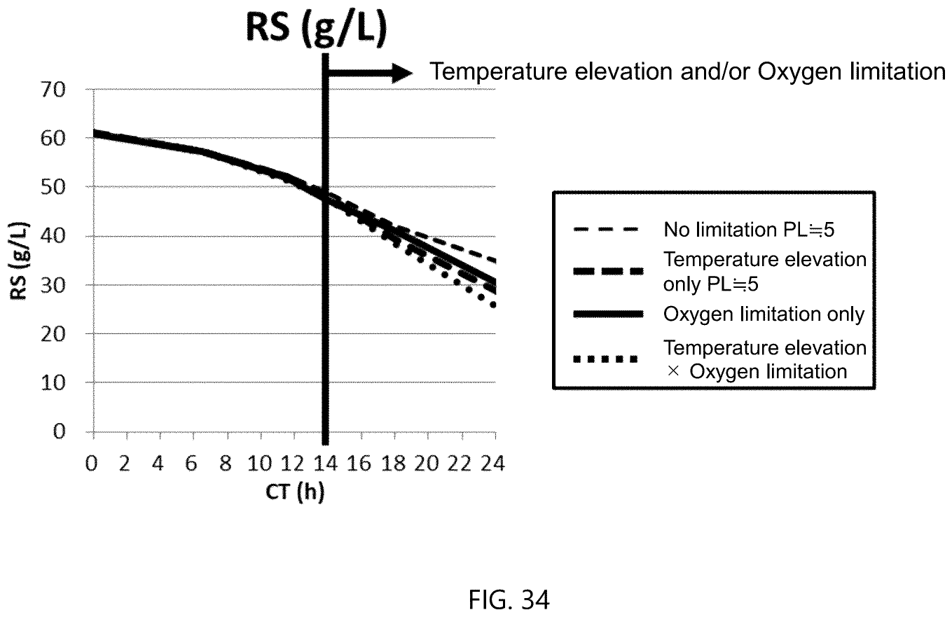

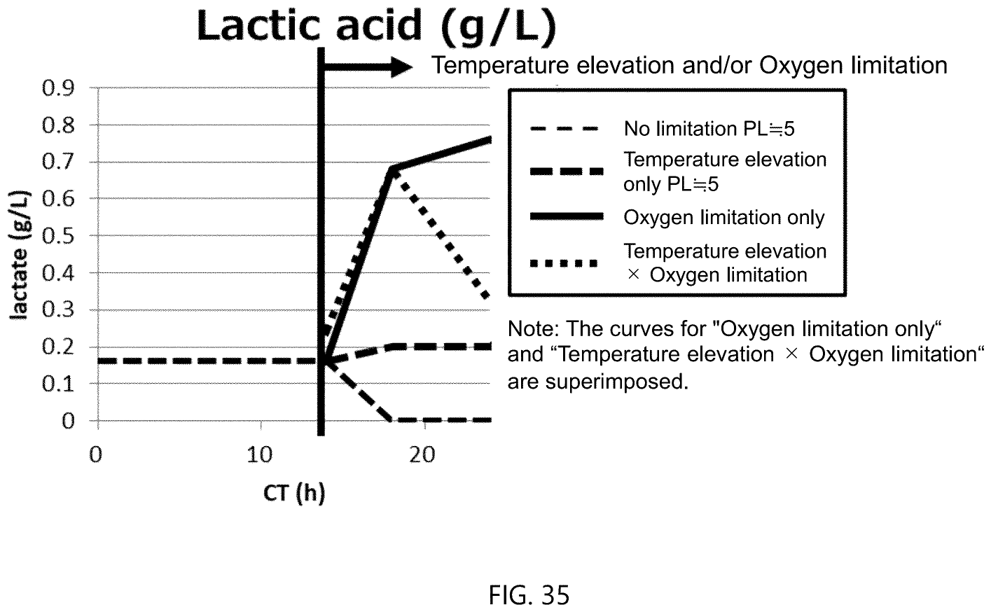

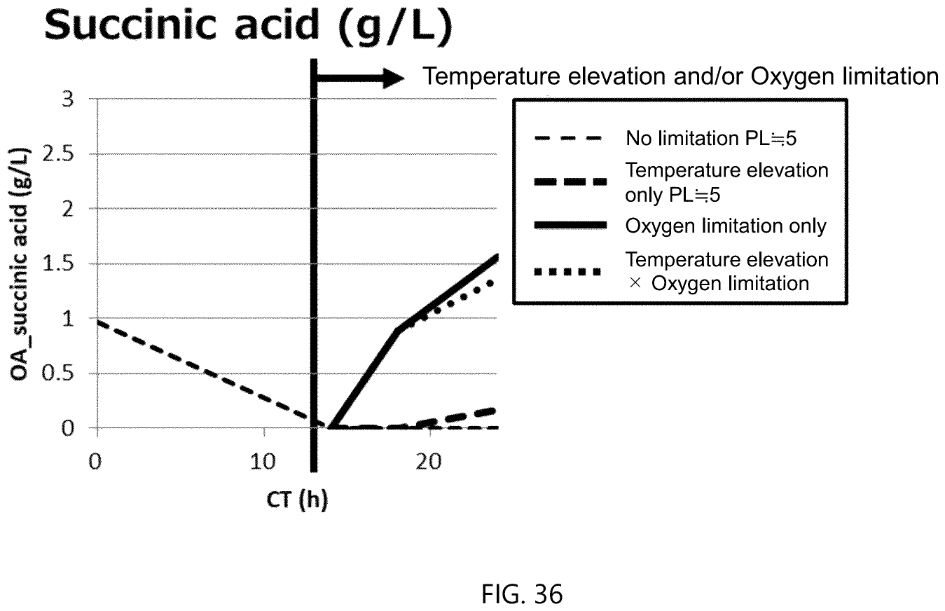

The results are shown in Table 11 and FIGS. 31 to 36. In C. casei WT+pVK9-ybjL strain, Glu production was hardly observed under the “no limitation” condition. In contrast, under the “temperature elevation only” or “oxygen limitation only” condition, Glu production increased. Furthermore, in C. casei WT+pVK9-ybjL strain, under the “temperature elevation×oxygen limitation” condition, Glu production increased significantly. Furthermore, under oxygen limitation conditions (“oxygen limitation only” and “temperature elevation×oxygen limitation” conditions), as compared to the “no limitation” condition, an increase in the accumulation of organic acids and a decrease in cell mass were confirmed.

This example further demonstrates that, in C. casei, oxygen limitation is effective for Glu production, and the combination of oxygen limitation and temperature elevation is particularly effective. Moreover, it was demonstrated that in the mutant strain of C. casei with enhanced Glu-producing ability compared to JCM 12072 (wild-type strain), a more significant increase in Glu production can be obtained by oxygen limitation or by the combination of oxygen limitation and temperature elevation.

TABLE 11

| Genus and | Corynebacterium casei | | | |
|---|---|---|---|---|
| Species | | | | |
| Strain | WT + pVK9-ybjL | | | |
| | | | | Temperature |
| | | Temperature | Oxygen | elevation × |
| Condition | No | elevation | limitation | Oxygen |
| or Purpose | limitation | only | only | limitation |
| Symbol | [symbol] | [symbol] | [symbol] | [symbol] |
| Yield (%) | 0.0 | 15.1 | 15.7 | 37.2 |
| 14 h~24 h | | | | |
| Glu conc. | 0.0 | 2.9 | 2.6 | 8.1 |
| (g/L) at 24 h | | | | |
| Residual | 35.0 | 28.9 | 30.7 | 25.7 |
| sugar | | | | |
| at 24 h | | | | |

### Example 6: Evaluation of the Impact of Increased Culture Temperature and/or Oxygen Limitation on Glu Production During Culture of C. glutamicum ATCC 13869 (Wild-Type Strain)

The impact of increased culture temperature and/or oxygen limitation on L-glutamic acid production was evaluated in C. glutamicum ATCC 13869 (wild-type strain), which is the most common Glu-producing bacterium. The increase of culture temperature is referred to simply as “temperature elevation”.

First, seed culture of ATCC 13869 was performed via jar fermentation using the seed culture medium shown in Table 6. The culture temperature was maintained constantly at 31.5° C., and the culture pH was controlled at 6.8 with ammonia gas.

Subsequently, the main culture of ATCC 13869 was performed via jar fermentation using the culture medium described in Table 7. The culture pH was maintained at 6.8 using ammonia gas, as with the seed culture. After inoculation with 15% seed culture broth to start the culture, the culture was conducted under the conditions of 31.5° C. and a relative dissolved oxygen concentration of 23% or more (air flow rate: ½ vvm, PL electrode reading of 5 or greater), until an OD620 of 30 was reached. When OD620 reached 30 (at 2 hours of cultivation), Tween 40 was added to a final concentration of 5 g/L, and the culture conditions were set to four different conditions: with or without oxygen limitation and with or without temperature elevation (changing the culture temperature from 31.5° C. to 36.5° C.), and cultivation was continued. The “no limitation” condition indicates that neither oxygen limitation nor temperature elevation was applied. The “temperature elevation only” condition refers to a condition in which oxygen limitation was not implemented, but temperature elevation was implemented. The “oxygen limitation only” condition refers to a condition in which temperature elevation was not implemented, but oxygen limitation was implemented. The “temperature elevation×oxygen limitation” condition refers to a condition in which both oxygen limitation and temperature elevation were implemented. When implementing oxygen limitation, the agitation speed of the jar fermenter was lowered, thereby inducing the PL electrode reading to close to zero. Sampling was performed over time during the culture.

Thus, four types of time-course culture broth samples containing L-glutamic acid were obtained. The amount of L-glutamic acid accumulated in the culture medium and the amount of glucose in the culture medium were measured using a biosensor BF-7 (Oji Scientific Instruments Co., Ltd.). The amount of organic acids accumulated in the medium was measured by liquid chromatography.

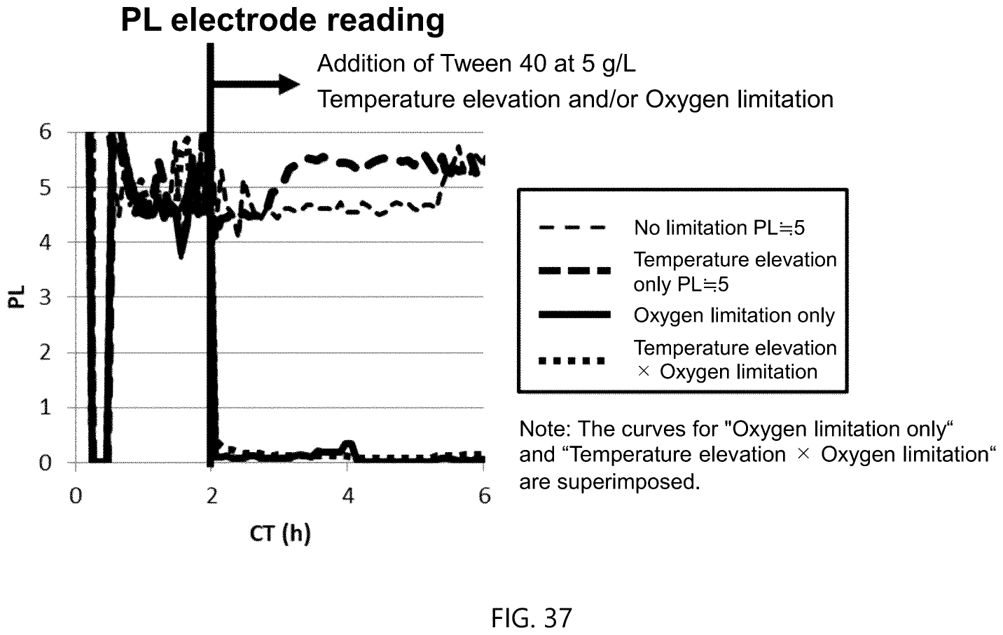

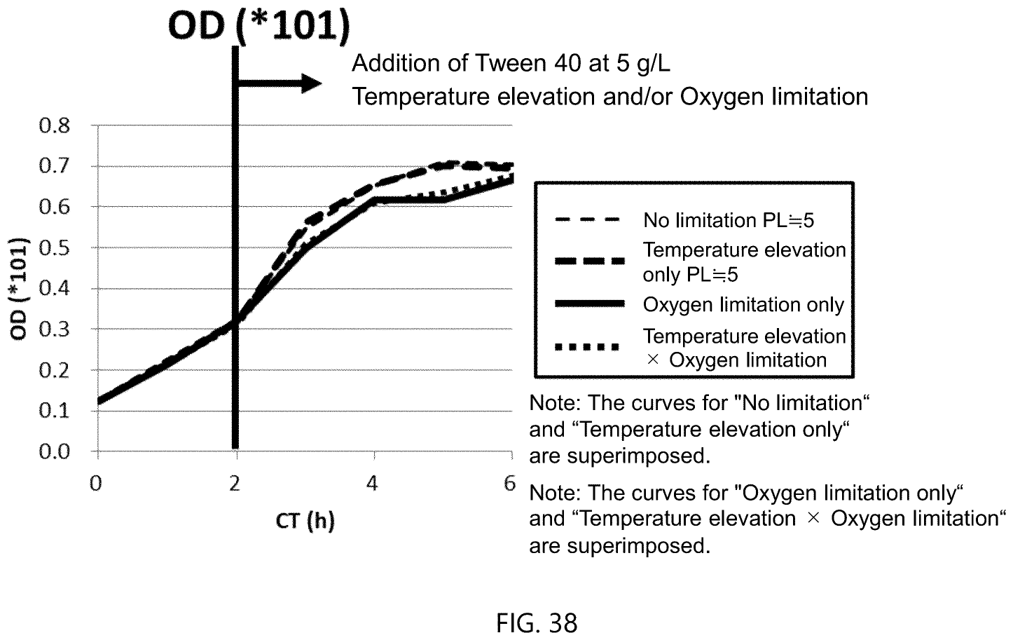

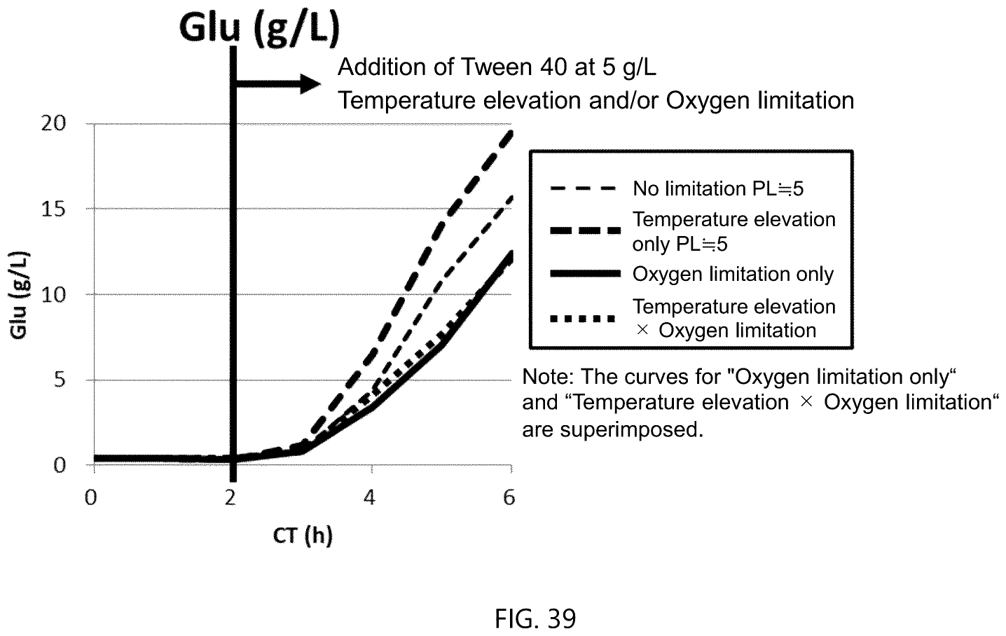

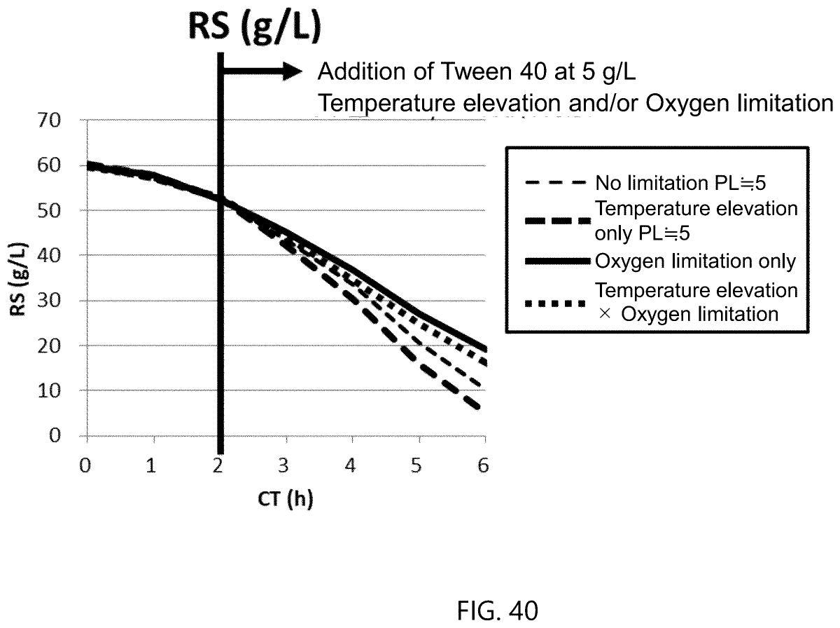

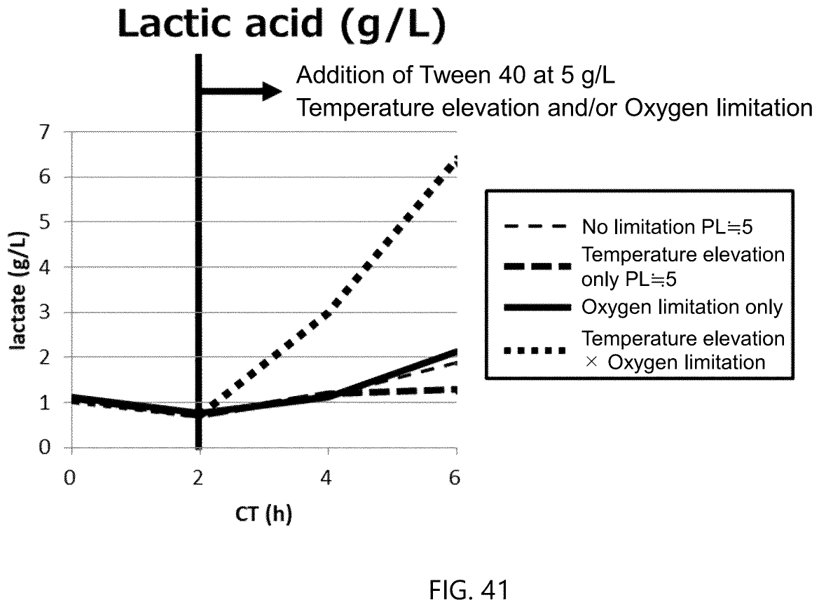

The results are shown in Table 12 and FIGS. 37 to 41. In C. glutamicum ATCC 13869 (wild-type strain), Glu production increased under the “temperature elevation only” condition compared to the “no limitation” condition; however, Glu production decreased under either the “oxygen limitation only” condition or the “temperature elevation×oxygen limitation” condition. These results exhibit the opposite effect to those confirmed in Examples 2 to 5 using various strains of C. casei, where enhancement of Glu production was observed through oxygen limitation or a combination of oxygen limitation and temperature elevation. Under oxygen limitation conditions (“oxygen limitation only” and “temperature elevation×oxygen limitation” conditions), as compared to the “no limitation” condition, an increase in the accumulation of organic acids and a decrease in cell mass were observed.

In this Example, it was demonstrated that, in C. glutamicum, oxygen limitation has a negative impact on Glu production, and even when combined with temperature elevation, a negative effect is observed, which is the opposite of the effects found in C. casei.

TABLE 12

| Genus and | Corynebacterium glutamicum | | | |
|---|---|---|---|---|
| Species | | | | |
| Strain | ATCC13869 | | | |
| | | | | Temperature |
| | | Temperature | Oxygen | elevation × |
| | No | elevation | limitation | Oxygen |
| Condition or | limitation | only | only | limitation |
| Purpose | (PL [symbol] 5) | (PL [symbol] 5) | (PL [symbol] 0) | (PL [symbol] 0) |
| Symbol | [symbol] | [symbol] | [symbol] | [symbol] |
| Yield (%) | 36.3 | 40.2 | 36.2 | 32.1 |
| (C.T. = 2~6) | | | | |

### Example 7: Evaluation of the Impact of Increased Culture Temperature and/or Oxygen Limitation on Glu Production During Culture of C. glutamicum with Enhanced Glu Exporter

It is known that Glu exporters play an important role in Glu fermentation. In particular, in C. glutamicum, enhanced Glu production has been confirmed even in the absence of additives such as surfactants like Tween 40 or ampicillin by expressing a mutant yggB gene (JP2007-097573A). Therefore, using C. glutamicum ATCC 13869-L30 strain (JP2007-097573A), which is a Glu-producing strain expressing the mutant yggB gene, the impacts of increased culture temperature and/or oxygen limitation on Glu production were evaluated. The increase in culture temperature is also referred to simply as “temperature elevation”.

First, seed culture of ATCC 13869-L30 strain was performed via jar fermentation using the seed culture medium shown in Table 6. The culture temperature was maintained constantly at 31.5° C., and the culture pH was controlled at 6.8 with ammonia gas.

Subsequently, the main culture of ATCC 13869-L30 strain was performed via jar fermentation using the culture medium described in Table 7. The culture pH was maintained at 6.8 using ammonia gas, as with the seed culture. After inoculation with 15% seed culture broth to start the culture, the culture was conducted under the conditions of 31.5° C. and a relative dissolved oxygen concentration of 23% or more (air flow rate: 1 vvm, PL electrode reading of 5 or greater), until an OD620 of 40 was reached. When OD620 reached 40 (at 3.5 hours of cultivation), the culture conditions were set to four different conditions: with or without oxygen limitation and with or without temperature elevation (changing the culture temperature from 31.5° C. to 36.5° C.), and cultivation was continued. The “no limitation” condition indicates that neither oxygen limitation nor temperature elevation was applied. The “temperature elevation only” condition refers to a condition in which oxygen limitation was not implemented, but temperature elevation was implemented. The “oxygen limitation only” condition refers to a condition in which temperature elevation was not implemented, but oxygen limitation was implemented. The “temperature elevation×oxygen limitation” condition refers to a condition in which both oxygen limitation and temperature elevation were implemented. When implementing oxygen limitation, the agitation speed of the jar fermenter was lowered, thereby inducing the PL electrode reading to close to zero. Sampling was performed over time during the culture.

Thus, four types of time-course culture broth samples containing L-glutamic acid were obtained. The amount of L-glutamic acid accumulated in the culture medium and the amount of glucose in the culture medium were measured using a biosensor BF-7 (Oji Scientific Instruments Co., Ltd.). The amount of organic acids accumulated in the medium was measured by liquid chromatography.

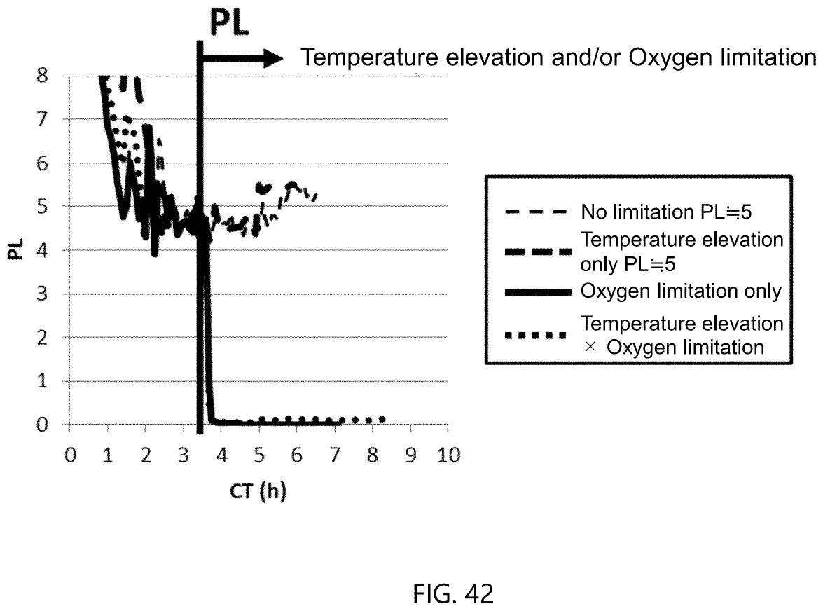

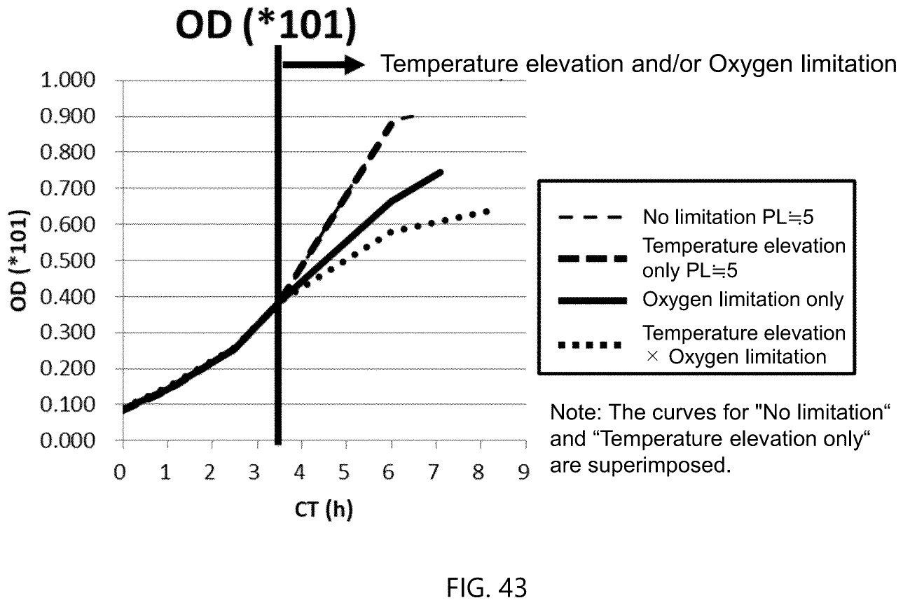

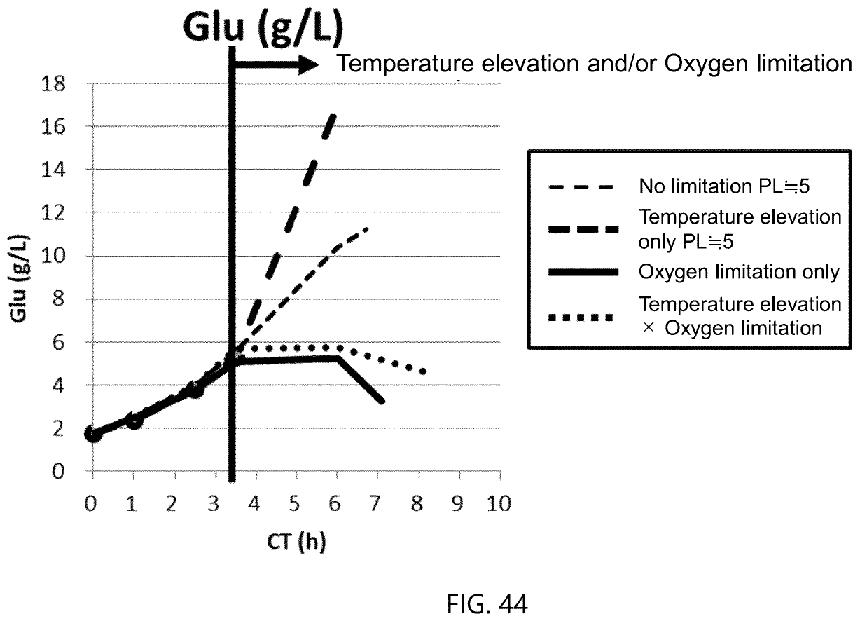

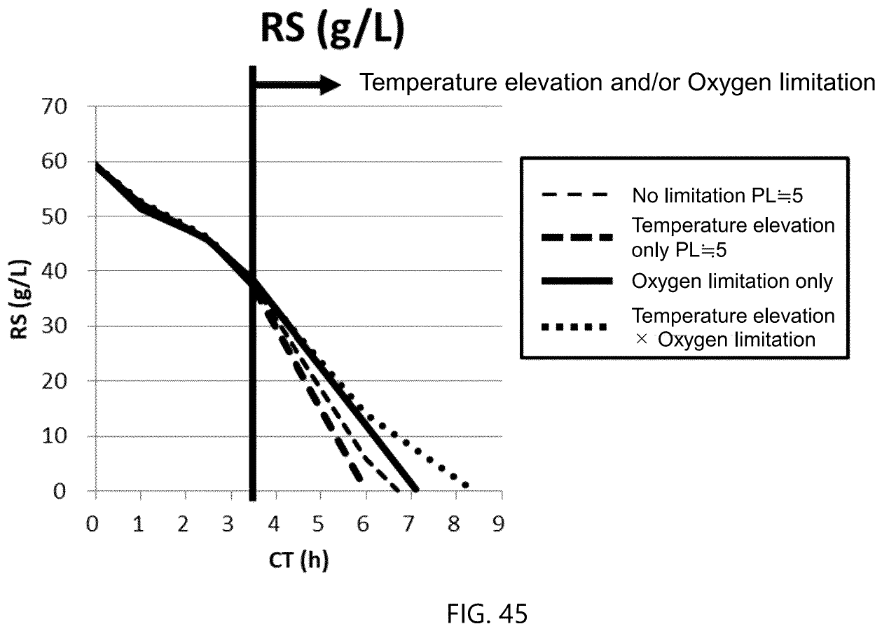

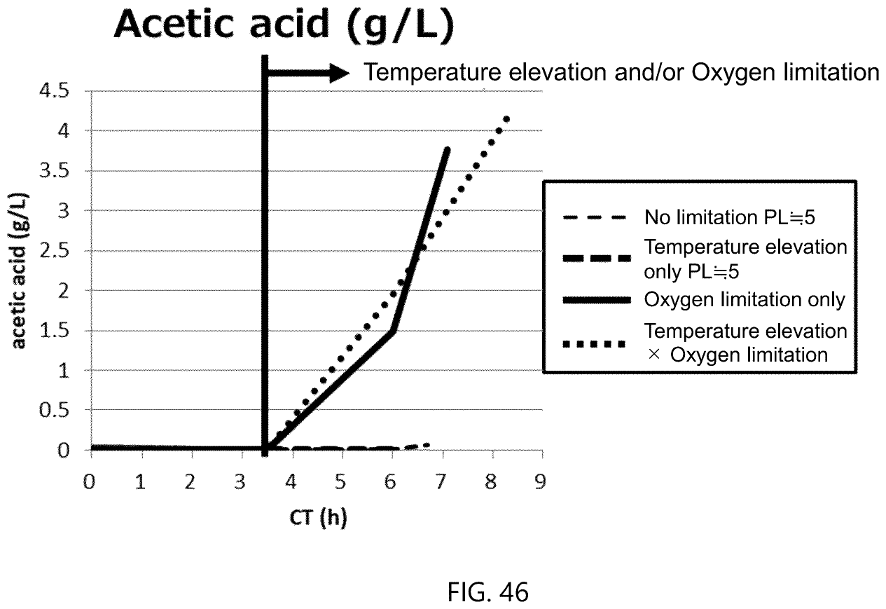

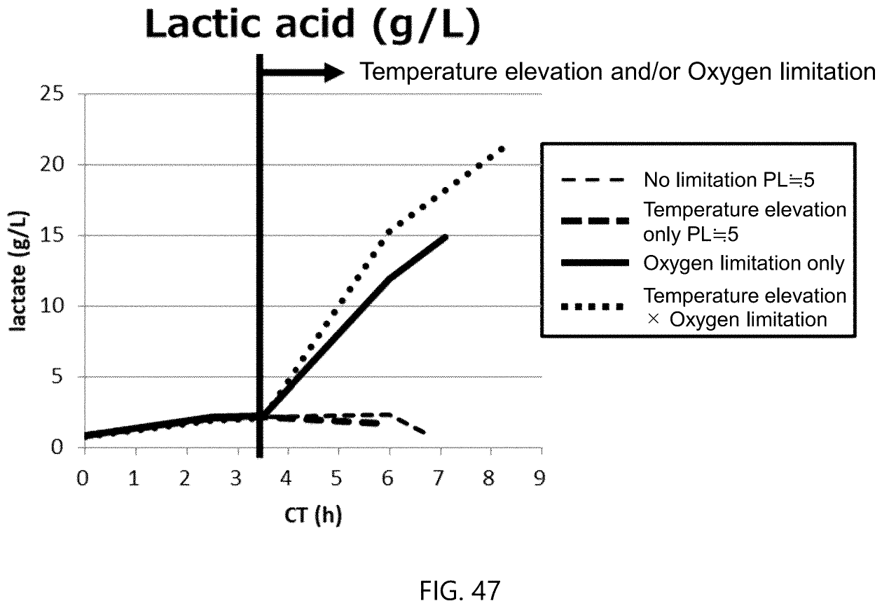

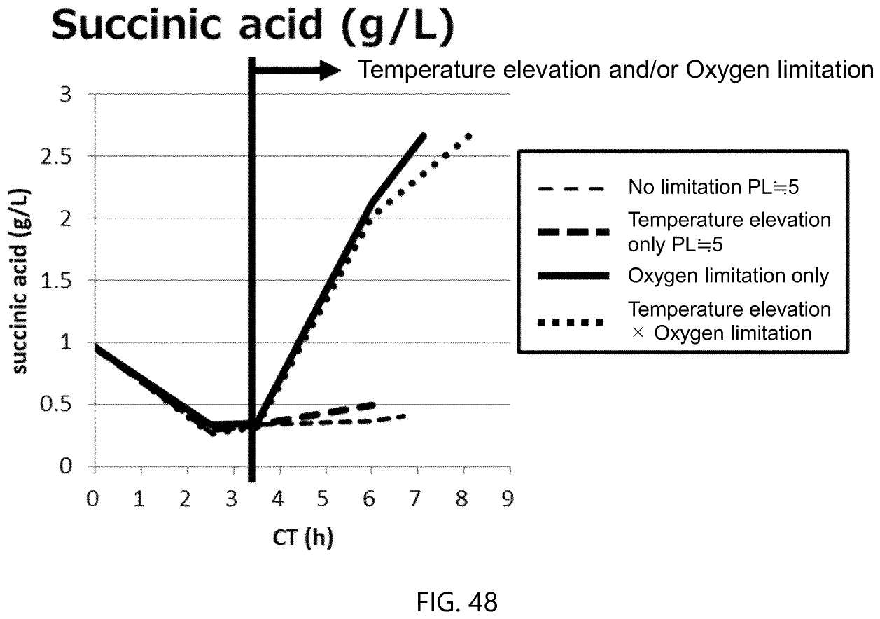

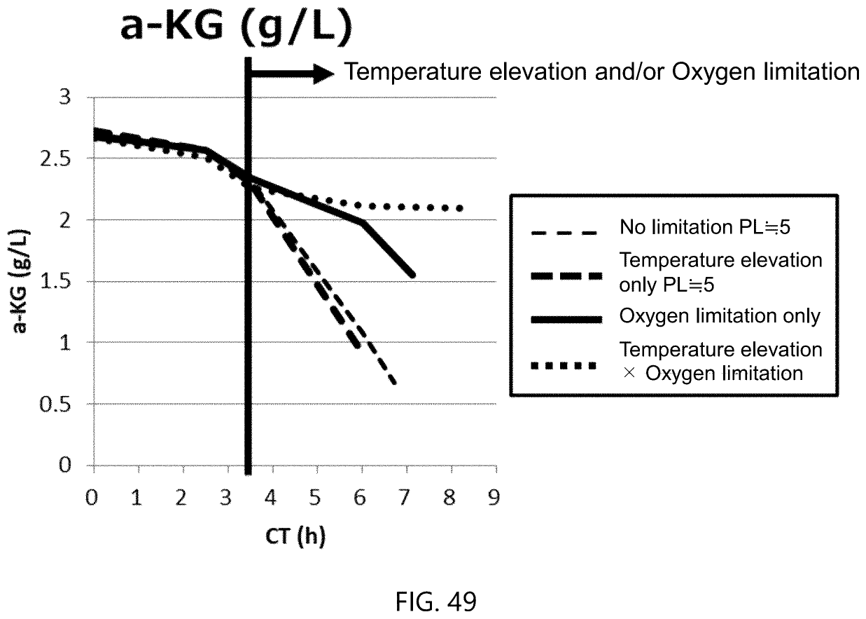

The results are shown in Table 13 and FIGS. 42 to 49. In C. glutamicum ATCC 13869-L30, Glu production increased under the “temperature elevation only” condition compared to the “no limitation” condition; however, Glu production decreased under either the “oxygen limitation only” condition or the “temperature elevation×oxygen limitation” condition. These results exhibit the opposite effect to those confirmed in Examples 2 to 5 using various strains of C. casei, where enhancement of Glu production was observed through oxygen limitation or a combination of oxygen limitation and temperature elevation. Under oxygen limitation conditions (“oxygen limitation only” and “temperature elevation×oxygen limitation” conditions), as compared to the “no limitation” condition, an increase in the accumulation of organic acids and a decrease in cell mass were observed.

In this Example, it was demonstrated that, in C. glutamicum, oxygen limitation has a negative impact on Glu production, and even when combined with temperature elevation, a negative effect is observed, which is the opposite of the effects found in C. casei.

TABLE 13

| Genus and | Corynebacterium glutamicum | | | |
|---|---|---|---|---|
| Species | | | | |
| Strain | ATCC13869-L30 | | | |
| | | | | Temperature |
| | | Temperature | Oxygen | elevation × |
| | No | elevation | limitation | Oxygen |
| Condition or | limitation | only | only | limitation |
| Purpose | (PL [symbol] 5) | (PL [symbol] 5) | (PL [symbol] 0) | (PL [symbol] 0) |
| Symbol | [symbol] | [symbol] | [symbol] | [symbol] |
| Yield (%) | 18.8 | 28.6 | 5.6 | 7.7 |
| BO-Glu conc. | 11.2 | 17.0 | 3.3 | 4.6 |
| (g/L) | | | | |
| Main CT (h) | 6.7 | 6.0 | 7.1 | 8.3 |

This application is a Continuation of PCT International Application No. PCT/JP2024/003374, filed Feb. 1, 2024, which is claiming priority of Japanese Patent Application No. 2023-014208, filed Feb. 1, 2023, all of which are hereby expressly incorporated by reference into the present application.

What is claimed is:

1. A method for producing L-glutamic acid, the method comprising:
a step of culturing a coryneform bacterium having an L-glutamic acid-producing ability in a culture medium to obtain a culture product containing L-glutamic acid,
wherein the bacterium is Corynebacterium casei, and
wherein oxygen limitation is implemented during said step.

2. The method according to claim 1, wherein the oxygen limitation is implemented so that the culture condition is a microaerobic condition.

3. The method according to claim 1, wherein the step comprises a culture period under an aerobic condition followed by a culture period under a microaerobic condition.

4. The method according to claim 1, wherein dissolved oxygen concentration in the culture medium during the period in which the oxygen limitation is implemented is less than 0.18 ppm.

5. The method according to claim 1, wherein the oxygen consumption rate during the period in which oxygen limitation is implemented is from 0.075 to 0.4 mol(O₂)/min/mL per OD620.

6. The method according to claim 1, wherein dissolved oxygen concentration in the culture medium during a period in which the oxygen limitation is not implemented is 0.18 ppm or more.

7. The method according to claim 1, wherein the oxygen limitation is implemented at a point when OD620 is 30 or more.

8. The method according to claim 1, wherein the length of the period in which the oxygen limitation is implemented is 1 hour or more.

9. The method according to claim 1, further comprising a step of increasing the temperature of the culture medium during said step.

10. The method according to claim 9, wherein an amount of said increase is from 1° C. to 12° C.

11. The method according to claim 9, wherein the temperature of the culture medium after said increase is from 32° C. to 40° C.

12. The method according to claim 9, wherein the temperature of the culture medium prior to said increase is from 20° C. to 32° C.

13. The method according to claim 9, wherein the increase is implemented at a point when OD620 is 30 or more.

14. The method according to claim 9, wherein the length for which the temperature after said increase is maintained is 1 hour or more.

15. The method as claimed in claim 1, wherein the L-glutamic acid is L-glutamic acid in a free form, sodium L-glutamate, potassium L-glutamate, ammonium L-glutamate, or a mixture thereof.

16. The method according to claim 1, wherein the L-glutamic acid is produced as a composition containing the L-glutamic acid.

17. The method according to claim 16, wherein the composition is a dried product of the culture product or a dried product of the supernatant of the culture product.

18. The method according to claim 1, further comprising a step of collecting the L-glutamic acid from the culture product.

19. The method according to claim 1, wherein the bacterium has one or more mutations selected from the mutations shown in Table 1 below
TABLE 1

| | | Base | Base |
|---|---|---|---|
| | Position | before | after |
| No. | on genome | mutation | mutation |
| A-1 | 78,486 | C | T |
| A-2 | 83,592 | G | A |
| A-3 | 87,955 | C | T |
| A-4 | 90,041 | C | T |
| A-5 | 186,221 | C | T |
| A-6 | 193,010 | C | T |
| A-7 | 196,531 | C | T |
| A-8 | 225,429 | C | T |
| A-9 | 297,920 | G | A |
| A-10 | 320,354 | C | T |
| A-11 | 335,878 | C | T |
| A-12 | 341,763 | C | T |
| A-13 | 346,969 | C | T |
| A-14 | 349,856 | C | T |
| A-15 | 356,232 | C | T |
| A-16 | 357,008 | C | T |
| A-17 | 366,674 | G | A |
| A-18 | 369,871 | G | A |
| A-19 | 377,420 | G | A |
| A-20 | 378,652 | G | A |
| A-21 | 432,252 | C | A |
| A-22 | 439,021 | G | A |
| A-23 | 440,764 | G | A |
| A-24 | 454,682 | G | A |
| A-25 | 458,729 | G | A |
| A-26 | 470,562 | G | A |
| A-27 | 471,288 | G | A |
| A-28 | 472,023 | G | A |
| A-29 | 504,885 | G | A |
| A-30 | 505,785 | G | A |
| A-31 | 514,371 | G | A |
| A-32 | 518,684 | G | A |
| A-33 | 521,126 | G | A |
| A-34 | 524,551 | G | A |
| A-35 | 660,841 | C | T |
| A-36 | 732,121 | C | T |
| A-37 | 787,055 | C | T |
| A-38 | 806,047 | C | T |
| A-39 | 872,482 | G | A |
| A-40 | 878,069 | C | T |
| A-41 | 903,037 | C | T |
| A-42 | 922,802 | C | T |
| A-43 | 948,145 | C | T |
| A-44 | 955,819 | C | T |
| A-45 | 968,915 | C | T |
| A-46 | 973,013 | C | T |
| A-47 | 974,797 | C | T |
| A-48 | 994,815 | C | T |
| A-49 | 1,000,498 | C | T |
| A-50 | 1,019,704 | C | T |
| A-51 | 1,049,052 | C | T |
| A-52 | 1,069,322 | C | T |
| A-53 | 1,070,554 | C | T |
| A-54 | 1,131,016 | C | T |
| A-55 | 1,138,639 | C | T |
| A-56 | 1,162,588 | C | T |
| A-57 | 1,193,273 | C | T |
| A-58 | 1,203,146 | C | T |
| A-59 | 1,222,633 | C | T |
| A-60 | 1,226,969 | G | A |
| A-61 | 1,264,895 | G | A |
| A-62 | 1,268,790 | G | A |
| A-63 | 1,279,676 | G | A |
| A-64 | 1,363,909 | T | C |
| A-65 | 1,387,476 | G | A |
| A-66 | 1,401,171 | G | A |
| A-67 | 1,416,228 | C | T |
| A-68 | 1,420,034 | C | T |
| A-69 | 1,447,494 | C | T |
| A-70 | 1,448,318 | C | T |
| A-71 | 1,448,776 | C | T |
| A-72 | 1,451,922 | C | T |
| A-73 | 1,466,961 | C | T |
| A-74 | 1,503,736 | C | T |
| A-75 | 1,504,207 | C | T |
| A-76 | 1,505,998 | C | T |
| A-77 | 1,507,027 | C | T |
| A-78 | 1,544,310 | C | T |
| A-79 | 1,554,973 | C | T |
| A-80 | 1,558,509 | C | T |
| A-81 | 1,562,459 | C | T |
| A-82 | 1,572,716 | C | T |
| A-83 | 1,594,314 | C | T |
| A-84 | 1,602,545 | C | T |
| A-85 | 1,659,808 | C | T |
| A-86 | 1,682,132 | C | T |
| A-87 | 1,689,863 | C | T |
| A-88 | 1,744,963 | C | T |
| A-89 | 1,784,642 | C | T |
| A-90 | 1,814,866 | C | T |
| A-91 | 1,829,145 | C | T |
| A-92 | 1,852,511 | G | A |
| A-93 | 1,861,170 | G | A |
| A-94 | 1,902,133 | G | A |
| A-95 | 1,916,048 | C | T |
| A-96 | 1,917,434 | C | T |
| A-97 | 1,938,271 | C | T |
| A-98 | 1,949,357 | G | T |
| A-99 | 1,954,368 | C | T |
| A-100 | 1,967,997 | C | T |
| A-101 | 1,975,599 | C | T |
| A-102 | 2,141,466 | C | T |
| A-103 | 2,308,064 | C | T |
| A-104 | 2,310,428 | C | T |
| A-105 | 2,354,420 | C | T |
| A-106 | 2,449,270 | T | C |
| A-107 | 2,449,278 | C | A |
| A-108 | 2,449,291 | G | C |
| A-109 | 2,449,318 | G | A |
| A-110 | 2,496,945 | C | T |
| A-111 | 2,505,022 | C | T |
| A-112 | 2,505,285 | C | T |
| A-113 | 2,525,513 | G | A |
| A-114 | 2,565,856 | C | T |
| A-115 | 2,601,306 | G | A |
| A-116 | 2,615,688 | G | A |
| A-117 | 2,650,740 | G | A |
| A-118 | 2,653,259 | G | A |
| A-119 | 2,663,827 | G | A |
| A-120 | 2,667,322 | G | A |
| A-121 | 2,674,077 | G | A |
| A-122 | 2,679,915 | G | A |
| A-123 | 2,686,979 | G | A |
| A-124 | 2,693,950 | C | T |
| A-125 | 2,696,737 | C | T |
| A-126 | 2,706,442 | C | T |
| A-127 | 2,709,469 | C | T |
| A-128 | 2,711,214 | C | T |
| A-129 | 2,714,651 | C | T |
| A-130 | 2,721,339 | G | A |
| A-131 | 2,731,030 | G | A |
| A-132 | 2,746,202 | G | A |
| A-133 | 2,805,389 | C | T |
| A-134 | 2,816,733 | G | A |
| A-135 | 2,827,114 | G | A |
| B-1 | 29,724 | G | A |
| B-2 | 92,869 | G | A |
| B-3 | 116,733 | G | A |
| B-4 | 131,184 | G | A |
| B-5 | 156,247 | G | A |
| B-6 | 177,083 | G | A |
| B-7 | 184,379 | G | A |
| B-8 | 212,586 | G | A |
| B-9 | 282,162 | G | A |
| B-10 | 309,483 | G | A |
| B-11 | 376,164 | C | T |
| B-12 | 440,885 | C | T |
| B-13 | 479,120 | G | A |
| B-14 | 722,430 | G | A |
| B-15 | 745,504 | G | A |
| B-16 | 809,993 | G | A |
| B-17 | 859,643 | G | A |
| B-18 | 923,209 | G | A |
| B-19 | 924,973 | G | A |
| B-20 | 998,893 | C | T |
| B-21 | 1,062,144 | C | T |
| B-22 | 1,095,062 | C | T |
| B-23 | 1,102,484 | C | T |
| B-24 | 1,103,812 | C | T |
| B-25 | 1,105,749 | C | T |
| B-26 | 1,107,561 | C | T |
| B-27 | 1,205,722 | C | T |
| B-28 | 1,233,449 | C | T |
| B-29 | 1,242,484 | C | T |
| B-30 | 1,248,388 | C | T |
| B-31 | 1,249,270 | C | T |
| B-32 | 1,291,377 | C | T |
| B-33 | 1,308,597 | C | T |
| B-34 | 1,329,535 | C | T |
| B-35 | 1,367,486 | C | T |
| B-36 | 1,382,065 | C | T |
| B-37 | 1,403,043 | C | T |
| B-38 | 1,433,914 | C | T |
| B-39 | 1,442,447 | C | T |
| B-40 | 1,501,903 | G | A |
| B-41 | 1,504,744 | C | T |
| B-42 | 1,651,403 | G | A |
| B-43 | 1,695,473 | G | A |
| B-44 | 1,779,939 | G | A |
| B-45 | 1,797,452 | G | A |
| B-46 | 1,801,284 | G | A |
| B-47 | 1,816,679 | G | A |
| B-48 | 1,832,252 | G | A |
| B-49 | 1,843,841 | G | A |
| B-50 | 1,868,285 | G | A |
| B-51 | 1,879,922 | G | A |
| B-52 | 1,892,007 | G | A |
| B-53 | 1,916,016 | G | A |
| B-54 | 1,937,604 | G | A |
| B-55 | 1,947,044 | G | A |
| B-56 | 1,948,411 | G | A |
| B-57 | 1,948,649 | G | A |
| B-58 | 1,967,697 | G | A |
| B-59 | 1,974,137 | G | A |
| B-60 | 2,028,284 | C | T |
| B-61 | 2,050,998 | G | A |
| B-62 | 2,052,708 | G | A |
| B-63 | 2,054,372 | G | A |
| B-64 | 2,065,568 | G | A |
| B-65 | 2,067,167 | G | A |
| B-66 | 2,082,577 | G | A |
| B-67 | 2,121,006 | A | G |
| B-68 | 2,149,369 | C | T |
| B-69 | 2,159,680 | G | A |
| B-70 | 2,380,965 | G | A |
| B-71 | 2,477,728 | G | A |
| B-72 | 2,542,800 | G | A |
| B-73 | 2,570,107 | G | A |
| B-74 | 2,647,383 | G | A |
| B-75 | 2,726,248 | C | T |
| B-76 | 2,825,055 | C | T |
| B-77 | 2,837,078 | C | T |
| B-78 | 2,865,322 | C | T |
| B-79 | 2,872,907 | C | T |
| B-80 | 2,880,351 | C | T |
| B-81 | 2,889,394 | C | T |
| B-82 | 2,906,471 | C | T |
| B-83 | 2,927,044 | C | T |
| B-84 | 2,929,963 | C | T |
| B-85 | 2,940,673 | C | T |
| B-86 | 2,946,285 | C | T |
| B-87 | 2,962,909 | C | T |
| B-88 | 2,975,742 | C | T |
| B-89 | 2,987,052 | C | T |
| B-90 | 3,079,560 | C | T |
| B-91 | 3,083,927 | C | T |
| B-92 | 3,090,163 | C | T |


SEQUENCE LISTING (4 sequences)

SEQ ID NO: 1 - DNA, 22 bp, synthetic construct
  source 1..22: mol_type = other DNA
  misc_feature 1..22: note = primer
atgttgaatgttaacatcgcag

SEQ ID NO: 2 - DNA, 37 bp, synthetic construct
  source 1..37: mol_type = other DNA
  misc_feature 1..37: note = primer
cggtacccggggatcttataagggcaataacggccag

SEQ ID NO: 3 - DNA, 38 bp, synthetic construct
  source 1..38: mol_type = other DNA
  misc_feature 1..38: note = primer
ccaagcttgcatgccatttgcgcctgcaacgtaggttg

SEQ ID NO: 4 - DNA, 38 bp, synthetic construct
  source 1..38: mol_type = other DNA
  misc_feature 1..38: note = primer
gttaacattcaacataacaggaatgttcctttcgaaaa